(19) United States
(12) Patent Application Publication
(10) Pub. No.: US 2025/0336532 A1
(43) Pub. Date: Oct. 30, 2025

(54) METHODS, SYSTEMS, AND COMPUTER READABLE MEDIA FOR IDENTIFYING BIOMARKERS INDICATIVE OF NEURODEGENERATION USING A COVARIANCE NEURAL NETWORK

(71) Applicant: The Trustees of the University of Pennsylvania, Philadelphia, PA (US)

(72) Inventors: Saurabh Sihag, Philadelphia, PA (US); Alejandro Rafael Ribeiro Pereira, Wynnewood, PA (US)

(21) Appl. No.: 19/195,070

(22) Filed: Apr. 30, 2025

Related U.S. Application Data
(60) Provisional application No. 63/640,679, filed on Apr. 30, 2024.

(51) Int. Cl. G16H 50/20

(52) U.S. Cl. CPC G16H 50/20

(57) ABSTRACT

A method for identifying biomarkers indicative of neurodegeneration using a covariance neural network (VNN) includes providing a VNN trained on brain anatomical data primarily composed of healthy subjects, making the largest proportion of the population in the data. Brain anatomical data of a subject is provided as input, and, based on the input, the VNN generates a set of biomarkers and a brain health marker indicative of neurodegeneration of the subject.

### PRIORITY CLAIM

This application claims the priority benefit of U.S. Provisional Patent Application Ser. No. 63/640,679, filed Apr. 30, 2024, the disclosure of which is incorporated herein by reference in its entirety.

### STATEMENT OF GOVERNMENT INTEREST

This invention was made with government support under 2031895 awarded by the National Science Foundation. The government has certain rights in the invention.

### TECHNICAL FIELD

The subject matter described herein relates to identifying neurodegeneration from brain imaging data using machine learning. More particularly, the subject matter described herein relates to methods, systems, and computer readable media for identifying biomarkers indicative of neurodegeneration using a covariance neural network.

### BACKGROUND

In computational neuroscience, there has been an increased interest in developing machine learning algorithms that leverage brain imaging data to provide estimates of “brain age” for an individual. Importantly, the discordance between brain age and chronological age (referred to as “brain age gap”) can capture accelerated aging due to adverse health conditions and therefore, can reflect increased vulnerability towards neurological disease or cognitive impairments. However, widespread adoption of brain age for clinical decision support has been hindered due to lack of transparency and methodological justifications in most existing brain age prediction algorithms.

Accordingly, there exists a need for a transparent (anatomically interpretable), generalizable, and robust machine learning framework for finding markers of neurodegeneration from neuroimaging data.

### SUMMARY

The subject matter described herein leverages coVariance neural networks (VNN) to propose an explanation-driven and anatomically interpretable framework for brain age prediction using cortical thickness features. Specifically, our brain age prediction framework extends beyond the coarse metric of brain age gap in Alzheimer's disease (AD) and we make two important observations: (i) VNNs can assign anatomical interpretability to elevated brain age gap in AD by identifying contributing brain regions, (ii) the interpretability offered by VNNs is contingent on their ability to exploit specific eigenvectors of the anatomical covariance matrix. Together, these observations facilitate an explainable and anatomically interpretable perspective to the task of brain age prediction.

Methods, systems, and computer readable media for identifying biomarkers indicative of neurodegeneration using a covariance neural network are disclosed. An example method for identifying biomarkers indicative of neurodegeneration using a covariance neural network (VNN) includes providing a VNN trained to predict features informative of the chronological age using the anatomical covariance matrix and the brain anatomical data derived from a population comprised primarily of healthy subjects that make the highest proportion. The method further includes providing, as input to the VNN, brain anatomical data of a subject and the anatomical covariance matrix, with the number of features in the brain anatomical data of the subject and the anatomical covariance matrix not necessarily the same as that for the brain anatomical data and the anatomical covariance matrix used for training the VNN. The method further includes generating, by the VNN and based on the input, a set of biomarkers indicative of neurodegeneration of the subject. The method further includes generating, based on the set of biomarkers generated by the VNN, a brain health marker indicative of neurodegeneration of the subject.

According to another aspect of the method described, the brain anatomical data used to train the VNN comprises multivariate dataset, whose each element captures a characteristic of a brain region or a combination of brain regions, and is derived from any combination of the following sources: magnetic resonance imaging (MRI) images, computed tomography (CT) scan, positron emission tomography (PET) scan, electroencephalogram (EEG) test of brains of a population comprised primarily of healthy subjects that makes up the largest proportion.

According to another aspect of the method described, the task for which the VNN is trained for comprises predicting chronological age or the features informative of the chronological age.

According to another aspect of the method described, the brain anatomical data of the subject comprises a multivariate dataset, whose each element captures a characteristic of a brain region or a combination of brain regions and is derived from any combination of the following sources: magnetic resonance imaging (MRI) images, computed tomography (CT) scan, positron emission tomography (PET) scan, electroencephalogram (EEG) test of brains.

According to another aspect of the method described, the brain anatomical data of the subject captures information about the same section of the brain as the brain anatomical data used to train the VNN, with the brain anatomical data of the subject and the brain anatomical data used to train the VNN not necessarily having the same number of features.

According to another aspect of the method described, the elements of the anatomical covariance matrix used to train the VNN are determined by the covariance between the features associated with different brain regions or a transformation of the covariance between the features associated with different brain regions. Examples of transformations on the covariance between the features associated with different brain regions can include thresholding, division, normalization, and multiplication.

According to another aspect of the method described, the elements of the anatomical covariance matrix used to process the brain anatomical data of the subject are determined by the covariance between the features associated with different brain regions or a transformation of the covariance between the features associated with different brain regions. Examples of transformations on the covariance between the features associated with different brain regions can include thresholding, division, normalization, and multiplication.

According to another aspect of the method described, the anatomical covariance matrix used to process the brain anatomical data of the subject may have different number of features relative to the anatomical covariance matrix used to train the VNN.

According to another aspect of the method described, generating the biomarker indicative of neurodegeneration of the subject includes the outputs of the VNN model or the statistical transformation of the outputs of the VNN model.

According to another aspect of the method described, generating the brain health marker indicative of neurodegeneration of the subject comprises determining, from the biomarkers, a prediction of the brain age of the subject.

According to another aspect of the method described, generating the brain health marker indicative of neurodegeneration of the subject comprises determining, from the biomarkers, a label of the subject among a category representing healthy population and one or more categories representing populations with neurodegenerative health conditions.

According to another aspect of the subject matter described, the method includes mapping the anatomical regions of the subject's brain to the biomarker.

According to another aspect of the method described, identifying the anatomical regions contributing to the brain age includes evaluating a statistic for an anatomical region from the biomarker that characterizes the anatomical region with respect to the brain age determined from the biomarkers.

According to another aspect of the method described, identifying the anatomical regions of the subject's brain contributing to the brain age comprises identifying the anatomical regions contributing to a prediction of brain age of the subject.

According to another aspect of the method described, identifying the anatomical regions contributing to the classification decision for a subject includes a statistical comparison of a biomarker for the subject relative to the biomarkers of a healthy population.

An example system for identifying biomarkers indicative of neurodegeneration using a covariance neural network (VNN) includes a computing platform including at least one processor, a memory, and a VNN trained exclusively on brain anatomical data from a dataset comprised primarily of healthy subjects, making up the largest proportion, with the VNN implemented by the at least one processor for receiving brain anatomical data of a subject as input. The VNN is further implemented for generating, based on the input, a set of biomarkers indicative of neurodegeneration of the subject. The VNN is further implemented for generating, based on the set of biomarkers, a brain health marker indicative of neurodegeneration of the subject.

According to another aspect of the system described, the brain anatomical data used to train the VNN comprises a multivariate dataset, whose each element captures a characteristic of a brain region or a combination of brain regions, and is derived from any combination of the following sources: magnetic resonance imaging (MRI) images, computed tomography (CT) scan, positron emission tomography (PET) scan, electroencephalogram (EEG) test of brains of a population comprised primarily of healthy subjects, making the largest proportion in the population.

According to another aspect of the system described, the elements of the anatomical covariance matrix used to train the VNN are determined by the covariance between the features associated with different brain regions or a transformation of the covariance between the features associated with different brain regions. Examples of transformations on the covariance between the features associated with different brain regions can include thresholding, division, normalization, and multiplication.

According to another aspect of the system described, generating the set of biomarkers indicative of neurodegeneration of the subject includes the outputs of the VNN or generating a statistical transformation of the outputs of the VNN.

According to another aspect of the system described, generating the brain health marker indicative of neurodegeneration of the subject includes a linear or non-linear transformation of the biomarkers.

According to another aspect of the system described, generating the brain health marker indicative of neurodegeneration of the subject includes generating brain age based on a linear or non-linear aggregation of the biomarkers.

According to another aspect of the system described, identifying the anatomical regions contributing to the brain age includes evaluating a residual vector for each anatomical region from the biomarker generated by the VNN that characterizes the anatomical region.

According to another aspect of the subject matter described, the system includes mapping anatomical regions of the subject's brain to the biomarkers.

According to another aspect of the subject matter described, the system includes identifying anatomical regions of the subject's brain contributing to the brain health marker.

According to another aspect of the system described, generating the brain health marker indicative of neurodegeneration of the subject includes prediction of the subject or the brain of the subject being healthy or unhealthy.

According to another aspect of the subject matter described, the system includes identifying anatomical regions of the subject's brain contributing to the prediction of the subject or the brain of the subject being healthy or unhealthy.

An example non-transitory computer readable medium has stored thereon a VNN trained exclusively on brain anatomical data from healthy subjects and executable instructions that when executed by at least one processor of at least one computer cause the at least one computer to perform steps including receiving brain anatomical data of a subject as input. The steps further include generating, based on the input, a set of biomarkers indicative of neurodegeneration of the subject. The steps further include generating, based on the input, a brain health marker indicative of neurodegeneration of the subject.

According to another aspect of the non-transitory computer readable medium described, generating the brain health marker indicative of neurodegeneration of the subject includes generating a prediction of brain age of the subject.

According to another aspect of the non-transitory computer readable medium described, generating the brain health marker indicative of neurodegeneration of the subject includes predicting the subject or the brain of the subject as healthy or unhealthy.

### Definitions

1. coVariance Neural Network (VNN): A VNN is a machine learning model whose architecture is derived from graph convolutional networks. This machine learning model operates on the sample covariance matrix estimated from a given dataset. VNN is inherently a scale-free model, i.e., its parameters are independent of the dimensionality of the dataset. Hence, a VNN trained on a dataset of a particular dimensionality can readily be transferred to process a dataset of another dimensionality with no change in the VNN architecture.
2. Stability of a Machine Learning Model: The notion of stability of a machine learning model pertains to the extent of the change in the model output with respect to the perturbations in the model input that arise from stochasticity of data acquisition. In this respect, VNNs are inherently stable models as they are robust to perturbations to the sample covariance matrix that was used in learning their parameters. Such perturbations may arise from situations like using different number of samples used to estimate the sample covariance matrix or estimation from an entirely different dataset that is sampled from the same underlying statistical distribution. Importantly, the stability of the VNNs adds confidence to the reproducibility of the findings reported using VNNs.
3. Foundation Model: In practice, a foundation model is a multi-purpose model that is trained on a vast amount of data and can be fine-tuned for specific tasks. A foundation model must be inherently ‘transferable’, i.e., it must be able to transfer the knowledge gained from the training task to provide informative outcomes on the desired task.
4. Brain Anatomical Data: Brain anatomical data refers to any multivariate datasets extracted from brain images (acquired during MRI, EEG, CT or PET scans) that describe the anatomical, physiological, metabolic and functional information about the brain. Example of such datasets include cortical morphology (e.g., thickness, volume, area) from structural MRI and BOLD time series data from functional MRI.
5. Biomarkers: Biomarker (or biological marker) are quantifiable characteristics of biological processes. In the context of this invention, the biomarker refers specifically to the outputs or the derived statistical transformation of the outputs of the VNN models, that can be used for downstream inference tasks including, but not limited to, classification of an individual as healthy or unhealthy and estimating biological age or brain age of an individual, and use brain activity till the current time step to predict brain activity in the next time step, etc.
6. Healthy Population: Healthy population broadly focuses on cognitively healthy population, generally characterized by no significant history of psychiatric disorder, substance abuse, neurological, or cardiovascular disease.

The subject matter described herein may be implemented in software in combination with hardware and/or firmware. For example, the subject matter described herein may be implemented in software executed by a processor. In one example implementation, the subject matter described herein may be implemented using a non-transitory computer readable medium having stored therein computer executable instructions that when executed by the processor of a computer control the computer to perform steps. Example computer readable media suitable for implementing the subject matter described herein include non-transitory devices, such as disk memory devices, chip memory devices, programmable logic devices, field-programmable gate arrays, and application specific integrated circuits. In addition, a computer readable medium that implements the subject matter described herein may be located on a single device or computer platform or may be distributed across multiple devices or computer platforms.

### BRIEF DESCRIPTION OF THE DRAWINGS

The subject matter described herein will now be explained with reference to the accompanying drawings of which:

FIG. 1 is a flow diagram illustrating the process for training and deploying a VNN model for identifying biomarkers and brain health marker indicative of neurodegeneration;

FIG. 2 illustrates a workflow for brain age and Δ-Age prediction using VNNs. Regions of interest (ROIs) contributing to elevated Δ-Age in neurodegeneration were identified by mapping the results of the analyses of the outputs at the final layer of VNNs on the brain surface;

FIG. 3 illustrates anatomically interpretable Δ-Age evaluation in OASIS-3 dataset. Panel a displays the robustness of the significantly elevated regional residuals for AD+ (Alzheimer's disease) group with respect to HC (healthy) group for different brain regions. For every VNN model in the set of 100 nominal VNN models that were trained on HC group, the analyses of regional residuals helped isolate brain regions that corresponded to significantly elevated regional residuals in AD+ group with respect to HC group. After performing this experiment for 100 VNN models, the robustness of the observed significant effects in a brain region was evaluated by calculating the number of times a brain region was identified to have significantly elevated regional residuals in AD+ group with respect to HC group. The number of times a brain region was linked with significantly elevated regional residuals in AD+ group with respect to HC group is projected on the brain template. Panel b displays the distribution of Δ-Age for HC and AD+ groups. The elevated brain age effect here is characterized by regional profile in Panel a. Panel c projects the mean Pearson's correlation between regional residuals (derived for each VNN model in the set of 100 nominal VNN models) and CDR (clinical dementia rating) sum of boxes for AD+ group on the brain template. Panel d displays the scatter plot for CDR sum of boxes versus Δ-Age in AD+ group. The correlation between Δ-Age and CDR sum of boxes could be attributed to the observations in Panel c;

FIG. 4, Panel a illustrates plots of the mean inner products between the normalized vector of regional residuals (norm=1) of VNN outputs (VNNs trained on OASIS-3) obtained from AD+ group and the eigenvectors of C_{HA} (covariance matrix from cortical thickness features) with respective standard deviations as error bars. Results with coefficient of variation >30% across the AD+ group have been excluded. Panel b illustrates the eigenvectors associated with the top three largest values for |<\tilde{r}_{AD+}, νᵢ>| are plotted on the brain surface. Subcallosal region in the right hemisphere was associated with the element with the largest magnitude in v₃ and v₄ and is highlighted with a red circle in the corresponding plots;

FIG. 5 illustrates a general overview of brain age evaluation using machine learning algorithms in the existing literature. Step 1 consists of training a machine learning (ML) model to predict chronological age (true age) for healthy controls. If the correlation between predicted age and true age is smaller than 1, an age-bias exists in ML model outputs as the age for older individuals tends to be under-estimated and that for younger individuals tends to be over-estimated. To correct for this bias, a linear regression based model is applied on the ML model outputs in step 2. Under the hypothesis that ML model can capture accelerated aging in neurodegeneration, it is expected that Δ-Age for individuals with neurodegeneration will be significantly higher than those of healthy controls (step 3);

FIG. 6 illustrates interpretability offered by VNNs in brain age prediction. By analyzing the final layer outputs of VNNs, we can isolate brain regions that have larger regional residuals for individuals with AD with respect to healthy controls. Furthermore, the elevated regional residuals in these brain regions eventually contribute to elevated Δ-Age after age-bias correction.

FIG. 7 illustrates an overview of the VNN architecture;

FIG. 8 illustrates the Inner product between the normalized vector of regional contributions to the VNN outputs P̅_{HC}) and eigenvectors of C_{H} (anatomical covariance matrix for HC group in OASIS-3). Panel a illustrates a bar plot for |<P̅_{HC}>νᵢ>| for i ∈{1, . . . , 30}, where νᵢ is the i-th eigenvector (principal component) of covariance matrix C_{H} and associated with i-largest eigenvalue in terms of magnitude and the vectors of regional contributions, P̅_{HC} were obtained by VNNs that were trained on OASIS-3 dataset. The inner product results for eigenvectors with coefficient of variation greater than 30% across the HC group of OASIS-3 were excluded (and hence, their respective entries set as 0). For every individual in HC group, the associations between their corresponding vector of regional contributions, P̅_{HC} and eigenvectors of C_{H} were evaluated over 100 nominal VNN models. The first eigenvector (v₁) had the largest association. The eigenvector v₁ is plotted on a brain template in Panel b;

FIG. 9 illustrates results depicting the brain regions with significantly elevated regional residuals for AD+ group with respect to HC group in OASIS-3. The results here were derived by a VNN model that was trained as a regression model to predict chronological age from cortical thickness data for HC group in OASIS-3. Box plots depicting the distributions of regional residuals in the HC and AD+ groups are shown for a few representative regions;

FIG. 10 illustrates, in Panel a, the distribution of Δ-Age across HC, MCI, Dementia cohorts derived from VNN models trained on OASIS-3. Anatomical covariance matrix from ADNI-1 dataset was used in the VNNs. A-Age for healthy controls: 0±3.92 years, Δ-Age for MCI (mild cognitive impairment) 3±5.74 years, Δ-Age for AD: 4.49±5.34 years. Panel b illustrates the distribution of Δ-Age across 100 VNNs that had been trained on OASIS-3 dataset, we evaluated the number of times the regional residual mean was smaller for HC group than the AD+ MCI group in ADNI-1 dataset;

FIG. 11 illustrates the distribution of chronological age in AD+ and HC groups;

FIG. 12 illustrates results of group differences in cortical thickness between AD+ and HC groups. Regions with significant differences (two-sided t-test, Bonferroni corrected p-value <0.05) are identified and the corresponding t-statistics are projected on a brain template. Negative t-statistic for a brain region suggests that the AD+ group had significant cortical atrophy in that region as compared to HC group;

FIG. 13 illustrates supplementary figures to FIG. 3. Panel a displays the plot of VNN prediction versus chronological age for HC group. VNN predictions were obtained as the average of the outputs of 100 nominal VNNs that were trained on OASIS-3 dataset and operated on the anatomical covariance matrix C_{HA}. Panel b displays the results similar to that in panel a for the AD+ group. The solid line in panels a and b is the least squares line. Panel c includes the boxplots for residuals derived from the difference between VNN predictions and chronological age for HC and AD+ groups. Panel d and e display the plots for brain age versus chronological age for HC and AD+ groups, respectively. The solid line in panels d and e is the identity line. Panel f displays the box plots for Δ-Age in HC and AD+ groups;

FIG. 14 illustrates results derived by VNN models that were randomly initialized and had the same architecture as those in Section 3.1;

FIG. 15 illustrates stability to perturbations in the anatomical covariance matrix for group differences between AD+ and HC groups observed in regional residuals. For a VNN model that was trained to predict chronological age for HC group in OASIS-3 dataset, the regional residuals were first determined using the anatomical covariance matrix C_{HA} formed by the cortical thickness data of complete OASIS-3 dataset (i.e., 611 HC individuals and 194 individuals in the AD+ group. We evaluated the F-values for the ANOVA test between regional residuals for AD+ group and HC group. The brain regions associated with the regional residuals that were significantly elevated in AD+ group with respect to HC group are highlighted on the brain template by projecting their F-values onto the relevant regions. The stability of the group differences to perturbations in C_{HA} was further investigated by varying the composition of cortical thickness data from AD+ and HC groups used to estimate C_{HA}. Figures in the top row display the results obtained via analysis of regional residuals by VNNs that processes the cortical thickness data from the complete OASIS-3 dataset over the anatomical covariance matrix C_{HA} estimated from 611 HC individuals and a varying number of individuals from the AD+ group. Figures in the bottom row illustrate the results of similar experiments, with the difference that the anatomical covariance matrix C_{HA} was estimated using all 194 individuals in the AD+ group and varying number of individuals from the HC group. The results corresponding to C_{HA} that was estimated using 194 AD+ individuals and 611 HC individuals formed the baseline for comparison for all scenarios;

FIG. 16 illustrates Δ-Age results for anatomical covariance matrix from only HC group (C_{H}). Panel a projects the robustness of observing a significantly higher regional residual for AD+ group with respect to HC group for a brain region on the template. Panel b plots the Δ-Age distributions for AD+ and HC groups derived from VNNs with C_{H} as the covariance matrix;

FIG. 17, Panel a, illustrates a bar plot for |<r̅_{AD+}, νᵢ>| for i ∈{1, . . . , 30}, where vᵢ is the i-th eigenvector of covariance matrix C_{H} associated with its i-largest eigenvalue. The bars are evaluated from the mean of |<r̅_{AD+}, νᵢ>| obtained for individuals in the AD+ group (results for eigenvectors associated with coefficient of variation of |<r̅_{AD+}, νᵢ>| larger than 30% excluded). For every individual in AD+ group, the association of its regional residuals with eigenvectors of C_{H} were evaluated over 100 nominal VNN models (trained on the OASIS-3 dataset). The eigenvectors associated with top three largest values for |<r̅_{AD+}, νᵢ>| are plotted on the brain template in Panel b illustrates that the subcallosal region in the right hemisphere was associated with the element with the largest magnitude in v₂ and v₃ and is highlighted with a red circle in the corresponding plots;

FIG. 18 is a flow diagram of an example method for identifying biomarkers indicative of neurodegeneration and a brain health marker indicative of neurodegeneration using a VNN;

FIG. 19 is a flow diagram illustrating an example process for applying the same VNN model for identifying biomarkers and brain age indicative of neurodegeneration in two distinct scenarios;

FIG. 20 illustrates representations of m-dimensional vector x and associated covariance matrix C in the continuous domain. a. Representation for x is obtained by discretizing the interval [0, 1]. b. Representation WC for C is obtained by discretizing the set [0, 1]² according to (18). Thus, WC retains the symmetry of C. The area spanned by the diagonal elements of C is marked in black. The size of the square area allotted to a diagonal element is proportional to its value. Other parts of the grid accommodate the off-diagonal elements of C;

FIG. 21 illustrates an overview of transferability of VNNs;

FIG. 22 illustrates cross-validation of Δ-Age and associated anatomical interpretability by leveraging the transferability of VNNs. If the transferability of parameters H for the chronological age prediction task holds for different datasets of m₁ and m₂ features, we expect to see similar Δ-Age distributions and associated anatomical interpretability (characterized by regions of interest (ROIs) highlighted in red color on the brain templates) across them without re-training; and

FIG. 23 illustrates brain age prediction across datasets curated according to multiple scales of Schaefer's atlas. Panel a illustrates the regional profiles consisting of brain regions with robust, elevated regional residuals in the combined AD+ group with respect to the HC group. The VNNs were trained to predict chronological age on FTDC100 and the robustness was evaluated over 100 nominal VNN models. The regional profiles were obtained for the datasets with 300 features and 500 features after transferring the VNNs from FTDC100 to FTDC300 and FTDC500. Panel b displays the box plots for Δ-Age corresponding to the regional profiles in Panel a. Panel c plots brain age versus chronological age for datasets with 100, 300, and 500 cortical thickness features.

### DETAILED DESCRIPTION

### 1. Introduction

Aging is characterized by progressive changes in the anatomy and function of the brain [1] that can be captured by different modalities of neuroimaging [2, 3]. Importantly, individuals can age at variable rates, a phenomenon described as “biological aging” [4]. Numerous existing studies based on a large spectrum of machine learning approaches study brain-predicted biological age, also referred to as brain age, which is derived from neuroimaging data [5]-[12]. Accelerated aging, i.e., when biological age is elevated as compared to chronological age (time since birth), may predict age-related vulnerabilities like risk for cognitive decline or neurological conditions like Alzheimer's disease (AD) [13, 14]. In this domain, the metric of interest is brain age gap, i.e., the difference between brain age and chronological age. We use the notation Δ-Age to refer to the brain age gap.

Inferring Δ-age from neuroimaging data presents a unique statistical challenge as it is essentially a qualitative metric with no ground truth and is expected to be elevated in individuals with underlying neurodegenerative condition as compared to the healthy population [12, 15]. The existing machine learning approaches for inferring Δ-Age commonly rely on regression models trained to predict chronological age for a healthy population. Under the hypothesis that such models can detect accelerated aging, they are applied to cohorts representing adverse health conditions. From a statistical perspective, the residuals of the regression models inform the Δ-Age estimates with the expectation that they will degrade in a specific direction when deployed to predict chronological age for individuals with adverse health conditions. Hence, it is paramount to analyze the structure and statistics of the residuals of the model to validate whether Δ-Age inferred using them provide biologically plausible information about the adverse health condition. A layman overview of the procedure of inferring Δ-Age is included in Appendix C. In this document, we focus on brain age prediction using cortical thickness features. Cortical thickness evolves with normal aging and is impacted due to neurodegeneration [17, 18]. Further, the age-related and disease severity related variations also appear in anatomical covariance matrices evaluated from the cortical thickness [19].

Existing literature. The current state-of-the-art deep learning methods in the brain age prediction domain focus exclusively on the performance of the model on predicting chronological age for a healthy population as a metric for assessing the quality of their approach [20-22]. We refer to such methods as performance-driven approaches to brain age prediction. Major criticisms of such performance-driven approaches include the coarseness of Δ-Age that results in lack of specificity of brain regions contributing to the elevated Δ-Age; and the lack of clarity regarding the reliance on the prediction accuracy for chronological age in the design of these brain age prediction models [5, 23].

To address the criticism regarding the lack of interpretability or explainability of Δ-Age, recent studies have utilized state-of-the-art post-hoc, model-agnostic methods, such as, SHAP, LIME [24], saliency maps [20, 25], and layer-wise relevance propagation [26] in conjunction with the performance-driven approaches. These methods commonly add anatomical interpretability to brain age estimates by assigning importance to the input features (often associated with specific anatomic regions). However, interpretability offered by such post-hoc approaches may not be conclusive if not shown to be stable to small perturbations to the input, variations in training algorithms and model multiplicity (i.e., when multiple models with similar performance may exist but offer distinct explanations) [27-29].

There exists sparse empirical evidence in the existing literature that hints at decoupling the task of brain age prediction from the performance achieved by the model in predicting chronological age for healthy population. For instance, a previous study has reported that models with a ‘moderate’ performance for predicting chronological age achieved a more informative brain age [21]. However, an appropriate ‘moderate’ fit on the chronological age that leads to the most informative brain age may not be generalizable to diverse datasets (diverse in terms of sample sizes, for example). Furthermore, a recent study of several existing brain age prediction frameworks has revealed that the accuracy achieved on the chronological age prediction task may not correlate with the clinical utility of associated Δ-Age estimates [30]. Intuitively, the performance on chronological age prediction task is an incomplete, if not flawed, metric for assessing the quality of Δ-Age estimate, as it cannot readily provide clarity on the correlation between the performance on predicting chronological age for healthy population and clinical utility of A-Age.

Explainable perspective to brain age prediction. In this document, we propose a principled framework for brain age prediction based on the recently studied coVariance neural networks (VNNs) [31]. VNN is a graph neural network (GNN) that operates on the sample covariance matrix as the graph and achieves learning objectives by manipulating the input data according to the eigenvectors (or principal components) of the covariance matrix. Thus, VNNs are inherently explainable models, as their inference outcomes can be linked with their ability to exploit the eigenvectors of the covariance matrix. In this context, the explainability offered by VNNs can be categorized as model-level explainability according to the taxonomy of explainability methods discussed in [32]. In general, model-level explainability can offer a more fundamental and generic understanding of the model than the aforementioned explainability methods applied in the brain age prediction application (such methods can broadly be categorized as instance-level methods [32]). A survey of explainability methods in GNNs is provided in Appendix B.

For the task of brain age prediction, one implementation of the subject matter described herein is not on the accuracy in predicting chronological age, but rather (i) what properties does a VNN gain when it is exposed to the information provided by chronological age of healthy controls, and (ii) whether and how these properties could translate to a meaningful brain age estimate. While highly relevant, these aspects are often overlooked in existing studies on brain age prediction. In this context, VNNs provide novel insights beyond that possible by focusing only on model performance. Specifically, training VNNs to predict chronological age using cortical thickness features from the healthy population fine-tuned their ability to exploit the eigenvectors of the anatomical covariance matrix. Further, the statistical analyses of the outputs of the final layer of the VNN allowed us to identify the most significant contributors to elevated Δ-Age in AD with respect to healthy population. Mapping these contributors on the brain surface rendered an anatomically interpretable perspective to Δ-Age estimates. Finally, the anatomical interpretability offered by VNNs to Δ-Age prediction in AD was strongly associated with certain eigenvectors of the covariance matrix, thus, rendering an explainable perspective to brain age in terms of the ability of VNNs to exploit the eigenvectors of the covariance matrix in a specific manner. We emphasize herein, the term ‘interpretability’ is used in the context of anatomic interpretability of Δ-Age and the term ‘explainability’ refers to explaining the VNN inference outcomes in terms of their associations with the eigenvectors of the covariance matrix.

The subject matter described herein relates to methods, systems, and computer readable media for identifying biomarkers indicative of neurodegenerative disease using a VNN. The described subject matter includes the development and implementation of the VNN as a foundation model for analyzing neuroimaging data. Foundation models are machine learning models trained on a representative dataset which can then be adopted for downstream tasks (e.g., ChatGPT is a well-known foundation model which was trained as a large language model and has been adopted for conversational usage). Here, we present a VNN-based paradigm for a foundation model that is trained on the data that primarily captures the characteristics of a healthy population and can further be fine-tuned to extract relevant biomarkers of clinical relevance as the downstream tasks.

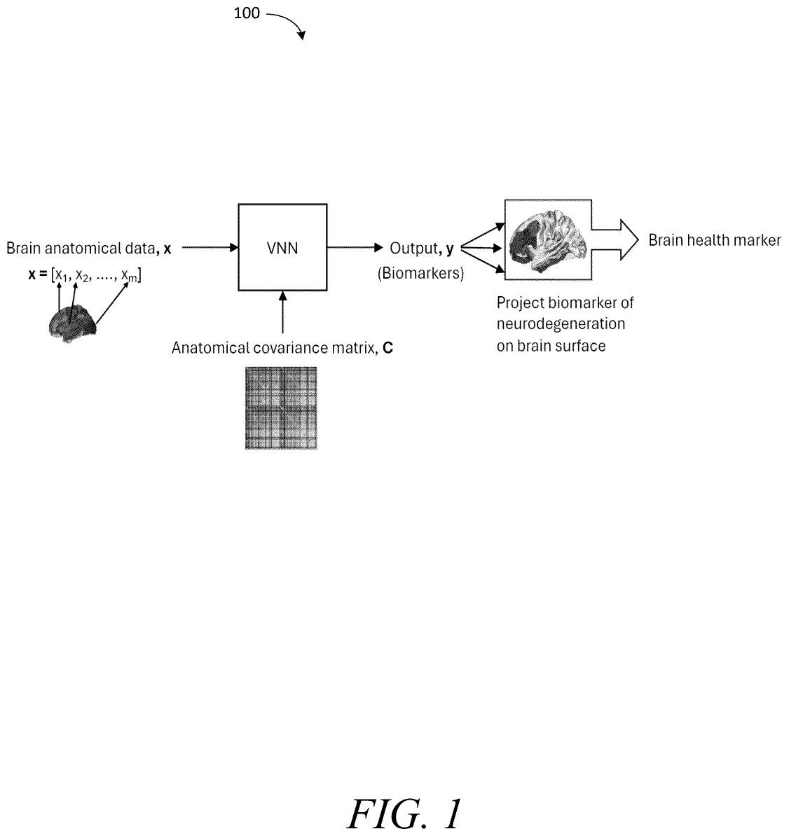

FIG. 1 is a flow diagram illustrating the process 100 for a VNN-based paradigm of identifying biomarkers and brain health marker of neurodegeneration from the brain anatomical data and associated anatomical covariance matrix. The inputs to the VNN are (i) the brain anatomical data from the subject, which is a multi-variate data sample, each of whose elements correspond to a feature associated with a distinct region or group of regions in the brain; and (ii) the anatomical covariance matrix, that captures the pair-wise associations between features associated with the brain regions. The VNN is pre-trained to predict chronological age or features related to chronological age on a dataset comprised primarily of healthy population in the largest proportion. For a subject, the VNN processes the inputs to generate a multi-variate output, which is a biomarker, or its mathematical transformation is a biomarker indicative of neurodegeneration in the subject. The biomarkers are associated with the brain regions and identify the brain regions with abnormal neurodegeneration (for example, pathological neurodegeneration relative to a healthy population). The biomarkers generated by the VNN are aggregated through linear or non-linear mathematical operations to generate a brain health marker. The brain health marker is indicative of neurodegeneration of the subject.

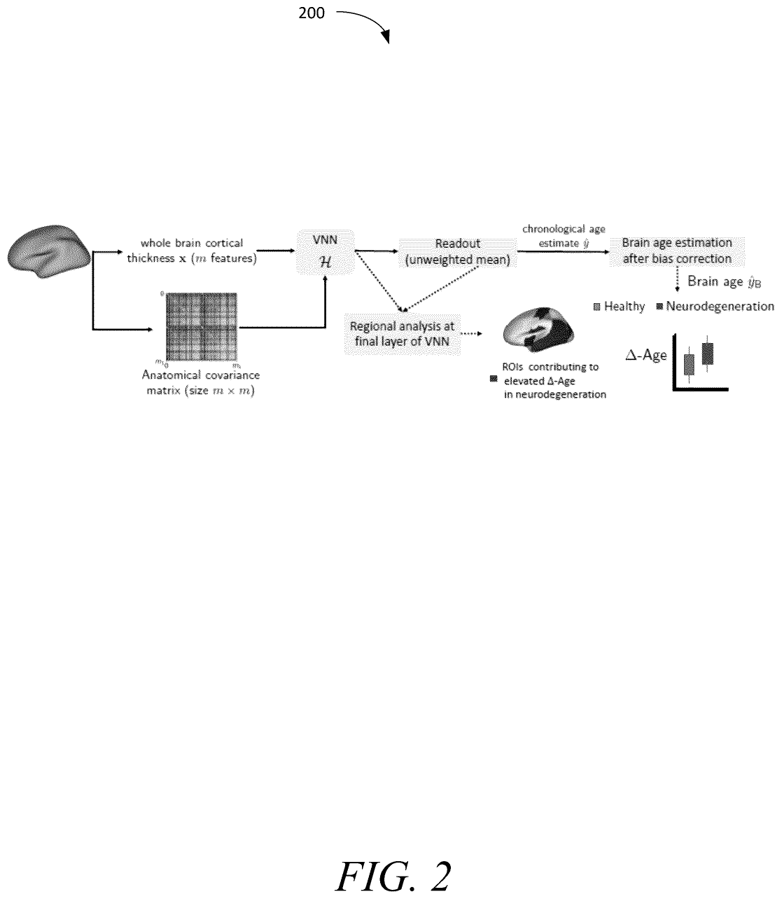

FIG. 2 is a flow diagram illustrating an example process 200 for training a VNN model for identifying biomarkers of a brain health marker indicative of neurodegeneration. In this example, VNN is trained to predict chronological age for healthy population using whole brain cortical thickness features and the associated anatomical covariance matrix. This pre-trained VNN model is leveraged to estimate the brain age as the brain health marker for a subject. Because a population with neurodegeneration, such as Alzheimer's disease, may exhibit accelerated biological aging including accelerated aging of the brain, by inputting to the VNN brain anatomical data of a subject with neurodegeneration, the VNN's output and the downstream tasks (such as regional analysis and bias correction) reflect an age greater than the true age of the subject. The outputs of the VNN generated from the subject's cortical thickness features are then aggregated and transformed to form the brain age of the subject. A brain age greater than the true age of the subject reflects accelerated biological aging and neurodegeneration in the subject. The Δ-Age is the difference between brain age and the true/chronological age of the subject.

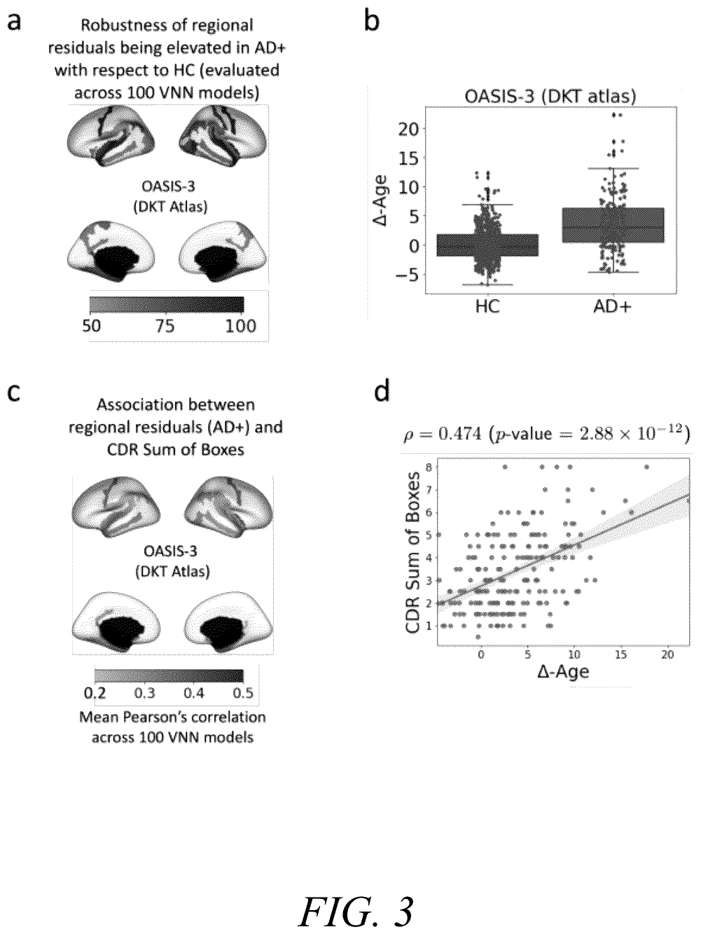

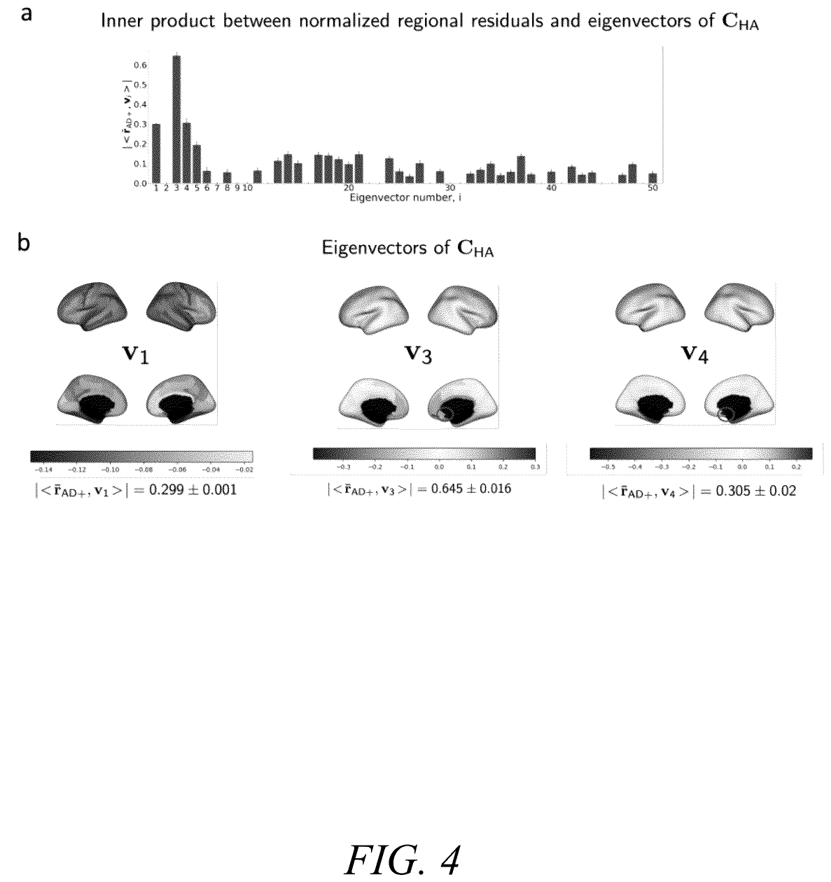

Example contributions of the subject matter described herein can be summarized as follows.
(a) VNNs provide anatomically interpretable Δ-Age: Δ-Age in individuals with AD diagnosis was elevated as compared to healthy controls and significantly correlated with a clinical marker of dementia severity. Moreover, by analyzing the outputs at the final layer of VNN for AD and healthy population and mapping the results on the brain surface, we could identify contributing brain regions to elevated Δ-Age in AD. Hence, the VNN architecture yielded anatomical interpretability for Δ-Age (FIG. 3).
(b) Anatomical interpretability correlated with eigenvectors of the anatomical covariance matrix: Our experiments demonstrated that certain eigenvectors of the anatomical covariance matrix were highly correlated with the features that facilitated anatomical interpretability for Δ-Age (FIG. 4). Thus, learning to predict chronological age of healthy population facilitated the ability of VNNs to exploit the eigenvectors of the anatomical covariance matrix that were relevant to Δ-Age, yielding an explainable perspective to brain age prediction.
   We focused our analysis on open access OASIS-3 dataset consisting of cortical thickness features from cognitively normal individuals and individuals in various stages of cognitive decline. The findings were further validated on the baseline ADNI-1 dataset.

### 2. coVariance Neural Networks

We begin with a brief introduction to VNNs. VNNs inherit the architecture of GNNs and operate on the sample covariance matrix as the graph. A dataset consisting of n random, independent and identically distributed (i.i.d) samples, given by xᵢ ∈ [symbol] ^{m×1}, ∀i ∈{1, . . . , n}, can be represented in matrix form as Xₙ=[x₁, . . . , xₙ]. Using Xₙ, the sample covariance matrix is estimated as

$$C \hat{=} \frac{1}{n - 1} {\sum }_{i = 1}^{n} ( {x}_{i} - \overline{x} ) ( {( {x}_{i} - \overline{x} )}^{T} \qquad ( 1 )$$

where x̅ is the sample mean of samples in Xₙ. The covariance matrix C can be viewed as the adjacency matrix of a graph representing the stochastic structure of the dataset Xₙ, where the m dimensions of the data can be thought of as the nodes of an m-node, undirected graph and its edges represent the pairwise covariances between different dimensions.

### Architecture

Similar to GNNs that rely on convolution operations modeled by linear-shift-and-sum operators, the convolution operation in a VNN is modeled by a coVariance filter, given by

$$H ( C ) \hat{=} {\sum }_{k = 0}^{K} {h}_{k} {C}^{k} , \qquad ( 2 )$$

where scalar parameters

$${\{ {h}_{k} \}}_{k = 0}^{K}$$

are referred to as filter taps that are learned from the data. The application of coVariance filter H(C) on an input x translates to combining information across different sized neighborhoods.

For K>1, the convolution operation combines information across multi-hop neighborhoods (up to K-hop) according to the weights hₖ to form the output z=H(C)x.

A single layer of VNN is formed by passing the output of the coVariance filter through a non-linear activation function σ(·) (e.g., Reule, \tanh) that satisfies σ(u)=[σ(u₁), . . . , σ(uₘ)] for u=[u₁, . . . , uₘ]. Hence, the output of a single layer VNN with input x is given by z=σ(H(C)x). The construction of a multi-layer VNN is formalized next.

Remark 1 (Multi-layer VNN). For an L-layer VNN, denote the coVariance filter in layer [symbol] of the VNN by [symbol] (C) and its corresponding set of filter taps by [symbol] Given a pointwise non linear activation function σ(·), the relationship between the input [symbol] and the output [symbol] or the [symbol] th layer is

$${x}_{ℓ} = σ ( {H}_{ℓ} ( C ) {x}_{ℓ - 1} ) for ℓ ∈ \{ 1 , … , L \} , \qquad ( 3 )$$

where x₀ is the input x.

Furthermore, similar to other deep learning models, sufficient expressive power can be facilitated in the VNN architecture by incorporating multiple input multiple output (MIMO) processing at every layer. Formally, consider a VNN layer [symbol] that can process [symbol] number of m-dimensional inputs and outputs [symbol] number of m-dimensional outputs via [symbol] × [symbol] number of filter banks. In this scenario, the input is specified as Xᵢₙ=[xᵢₙ[1], . . . , xᵢₙ[Fₙ]], and the output is specified as Xₒᵤₜ=[xₒᵤₜ[1], . . . , xₒᵤₜ[Fₒᵤₜ]]. The relationship between the f-th output xₒᵤₜ[f] and the input xᵢₙ is given by

$${x}_{out} [ f ] = σ ( {\sum }_{g = 1}^{{F}_{in}} {H}_{fg} ( C ) {x}_{i n} [ g ] ,$$

where H_{fg}(C) is the coVariance filter that processes xᵢₙ[g]. Without loss of generality, we assume that [symbol] =F, ∀ [symbol] ∈{1, . . . , L}. In this case, the set of all filter taps is given by

$$H = \{ {H}_{f g}^{ℓ} \} , ∀ f , g ∈ \{ 1 , … , F \} , ℓ ∈ \{ 1 , … , L \} ,$$

where

$${ℋ}_{fg} = {\{ {h}_{fg}^{ℓ} [ k ] \}}_{k = 0}^{K} and {h}_{fg}^{ℓ} [ k ]$$

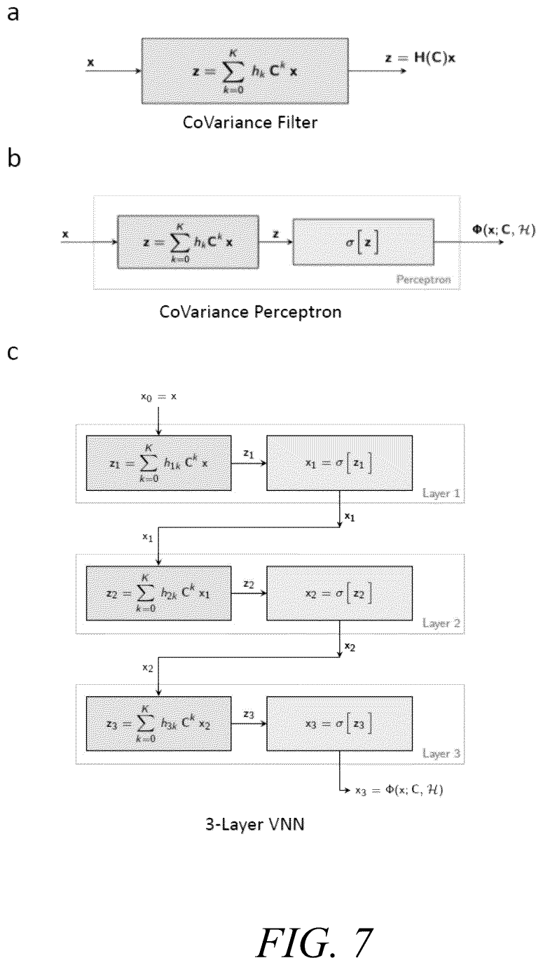

is the k-th filter tap for filter H_{fg}(C). Thus, we can compactly represent a multi-layer VNN architecture capable of MIMO processing via the notation Φ(x; C, H), where the set of filter taps [symbol] captures the full span of its architecture. We also use the notation Φ(x; C, Hi) to denote the output at the final layer of the VNN. Various aspects of the VNN architecture are illustrated in FIG. 7 referenced in Appendix D. The VNN final layer output Φ(x; C, H) is succeeded by a readout function that maps it to the desired inference outcome.

Remark 2 (Statistical inference using VNNs). Given the eigendecomposition of C=VΛV^{T}, the spectral properties of the VNN are established by studying the projection of the coVariance filter output z=H(C)x on the eigenvectors V (similar to that for a graph filter using graph Fourier transform [39, 40]). Theorem 1 in [31] established the equivalence between processing data samples with principal component analysis (PCA) transform and processing data samples with a specific polynomial on the covariance matrix C. Hence, it can be concluded that input data is processed with VNNs, at least in part, by exploiting the eigenvectors of C. Unlike simpler PCA-based inference models, VNNs offer stability [31] and transferability guarantees [35], which ensure reproducibility of the inference outcomes by VNNs with high confidence.

In the context of brain age prediction, we leverage the observations in Remark 2 to demonstrate the relationships of the inference outcomes with the eigenvectors of the anatomical covariance matrix C (estimated from cortical thickness features) in Section 4.

### 2.2 VNN Learning

The VNN model is trained for a regression task, where the chronological age is predicted using m cortical thickness features. Since the VNN architecture has F number of m-dimensional outputs in the final layer, Φ(x; C, [symbol] ) is of dimensionality m×F. The regression output is determined by a readout layer, which evaluates an unweighted mean of all outputs at the final layer of VNN. Therefore, the regression output for an individual with cortical thickness x is given by

$$\hat{y} = \frac{1}{Fm} {\sum }_{j = 1}^{m} {\sum }_{f = 1}^{F} {[ Φ ( x ; C , ℋ ) ]}_{jf} . \qquad ( 4 )$$

Prediction using unweighted mean at the output implies that the VNN model exhibits permutation-invariance (i.e., the final output is independent of the permutation of the input features and covariance matrix). Moreover, it allows us to associate a scalar output with each brain region among the m regions at the final layer. Specifically, we have

$$p = \frac{1}{F} {\sum }_{f = 1}^{F} {[ Φ ( x ; C , ℋ ) ]}_{f} , \qquad ( 5 )$$

where p is the vector denoting the mean of filter outputs in the final layer's filter bank. Note that the mean of all elements in p is the prediction ŷ formed in (4). In the context of cortical thickness datasets, each element of p can be associated with a distinct brain region. Therefore, p is a vector of “regional contributions” to the output ŷ by the VNN. This observation will be instrumental to establishing the interpretability offered by VNNs in the context of Δ-Age prediction in Section 3. For a regression task, the training dataset

$${\{ {x}_{i} , {y}_{i} \}}_{i = 1}^{n}$$

(where xᵢ are the cortical thickness data for an individual i with chronological age yᵢ) is leveraged to learn the filter taps in [symbol] for the VNN Φ(·; C, [symbol] ) such that they minimize the training loss, i.e.,

$${H}_{opt} = {min}_{H} \frac{1}{n} {\sum }_{i = 1}^{n} ℓ ( {\hat{y}}_{i} , {y}_{i} ) , \qquad ( 6 )$$

where ŷᵢ is evaluated similarly to (4) and [symbol] (·) is the mean-squared error (MSE) loss function.

### 3. Methods Overview for Brain Age Prediction

In this section, we provide an overview of the brain age prediction framework based on VNNs (see FIG. 2 for a summary). Our results primarily focus on the dataset described below.

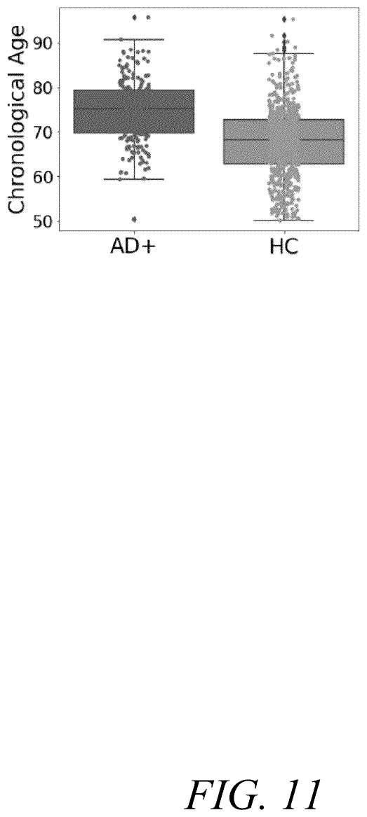

OASIS-3 Dataset. This dataset was derived from publicly available freesurfer estimates of cortical thickness (derived from MRI images collected by 3 Tesla scanners and hosted on central.xnat.org), as previously reported [33], and comprised of cognitively normal individuals (HC; n=611, age=68.38±7.62 years, 351 females) and individuals with AD dementia diagnosis and at various stages of cognitive decline (n=194, age=74.72+7.02 years, 94 females). The cortical thickness features were curated according to the Destrieux (DKT) atlas [41](consisting of m=148 cortical regions). For clarity of exposition of the brain age prediction method, any dementia staging to subdivide the group of individuals with AD dementia diagnosis into mild cognitive impairment (MCI) or AD was not performed, and we use the label AD+ to refer to this group. The individuals in AD+ group were significantly older than those in HC group (t-test: p-value=2.46×10⁻²³). The boxplots for the distributions of chronological age for HC and AD+ groups are included in FIG. 11 described in Appendix I. For 191 individuals in the AD+ group, the clinical dementia rating (CDR) sum of boxes scores evaluated within one year (365 days) from the MRI scan were available (CDR sum of boxes=3.45±1.74). CDR sum of boxes scores are commonly used in clinical settings to stage dementia severity and were evaluated according to [43].

Cross-validation. The findings obtained via the analyses of the OASIS-3 dataset were cross-validated on the ADNI-1 dataset (described in Appendix H).

### 3.1 Training the VNNs on HC Group

We first train the VNN model to predict chronological age using the cortical thickness features for the HC group. This enables the VNN models to capture the information about healthy aging from the cortical thickness and associated anatomical covariance matrix. The hyperparameters for the VNN architecture and learning rate of the optimizer were chosen according to a hyperparameter search procedure [44]. The VNN model had L=2-layers with a filter bank, such that we had F=5, and 6 filter taps in the first layer and 10 filter taps in the second layer. The learning rate for the Adam optimizer was set to 0.059. The number of learnable parameters for this VNN was 290. The HC group was split into a 90/10 training/test split, and the covariance matrix was set to be the anatomical covariance evaluated from the training set. A part of the training set was used as a validation set and the other used for training the VNN model. We trained 100 VNN models, each on a different permutation of the training data. The training process was similar for all VNNs and is described in Appendix E. No further training was performed for the VNN models in the subsequent analyses.

### 3.2 Analyses of Regional Residuals in AD+ and HC Groups

Next, the VNN models trained to predict the chronological age for the HC group and [5] were adopted to study the effect of neurodegeneration on brain regions for AD+ group. Since the impact of neurodegeneration was expected to appear in the anatomical covariance matrix, we report the results when anatomical covariance C_{HA} from the combined cortical thickness data of HC and AD+ groups was deployed in the trained VNN models. Because of the stability property of VNNs [31, Theorem 3], the inference drawn from VNNs was expected to be stable to the composition of combined HC and AD+ groups used to estimate the anatomical covariance matrix C_{HA}.

For every individual in the combined dataset of HC and AD+ groups, we processed their cortical thickness data x through the model Φ(x; C_{HA}, [symbol] ) where parameters [symbol] were learned in the regression task on the data from HC group as described previously. Hence, the vector of mean of all final layer outputs for cortical thickness input x is given by

$$p = \frac{1}{F} {{\sum }_{f}^{F} [ Φ ( x ; {C}_{HA} , ℋ ]}_{f}$$

and the VNN output is

$$\hat{y} = \frac{1}{148} {{\sum }_{j = 1}^{148} [ p ]}_{j} .$$

Furthermore, we define the residual for feature α (or brain region represented by feature α in this case) as

$${[ r ]}_{a} {=}^{Δ} {[ p ]}_{a} - \hat{y .} \qquad ( 7 )$$

Thus, (7) allows us to characterize the residuals with respect to the VNN output ŷ at the regional level, where brain regions are indexed by a. Henceforth, we refer to the residuals (7) as “regional residuals”. Recall that these are evaluated for an individual with cortical thickness data x. We hypothesized accelerated aging for an individual to be an aggregated effect of contributions from certain biologically plausible brain regions. The brain regions contributing to the observed higher Δ-Age (procedure described in Section 3.3) could be characterized at a regional level by the analysis of regional residuals as defined in (7). Thus, the elements of the residual vector r can potentially act as a biomarker that can enable the isolation of brain regions impacted due to accelerated aging in AD.

In our experiments, for a given VNN model, the residual vector r was evaluated for every individual in the OASIS-3 dataset. Also, the population of residual vectors for the HC group is denoted by r_{HC}, and that for individuals in the AD+ group by r_{AD+}. The length of the residual vectors is the same as the number of cortical thickness features (i.e., m=148). Further, each element of the residual vector was mapped to a distinct brain region and ANOVA was used to test for group differences between individuals in HC and AD+ groups. Also, since elevation in Δ-Age is the biomarker of interest in this analysis, we hypothesized that the brain regions that exhibited higher means for regional residuals for AD+ group than HC group would be the most relevant to capturing accelerated aging. Hence, the results are reported only for brain regions that showed elevated regional residual distribution in AD+ group with respect to HC group. Further, the group difference between AD+ and HC groups in the residual vector element for a brain region was deemed significant if it met the following criteria: i) the corrected p-value (Bonferroni correction) for the clinical diagnosis label in the ANOVA model was smaller than 0.05; and ii) the uncorrected p-value for clinical diagnosis label in ANCOVA model with age and sex as covariates was smaller than 0.05. See Appendix G for an example of this analysis.

Recall that 100 distinct VNNs were trained as regression models on different permutations of the training set of cortical thickness features from HC group. We used these trained models to establish the robustness of observed group differences in the distributions of regional residuals.

Deriving anatomical interpretability or regional profile for Δ-Age from the robustness of findings from regional analyses. We performed the regional analysis described above corresponding to each trained VNN model and tabulated the number of VNN models for which a brain region was deemed to be associated with a significantly elevated regional residual for the AD+ group. A larger number of VNN models isolating a brain region as significant suggested that this region was likely to be a highly robust contributor to accelerated aging in the AD+ group.

Explaining the anatomical interpretability. We further investigated the relationship between regional residuals derived from VNNs and the eigenvectors of C_{HA} to determine the specific eigenvectors (principal components) of C_{HA} that were instrumental to anatomical interpretability. For this purpose, we evaluated the inner product of normalized residual vectors (norm=1) obtained from VNNs and the eigenvectors of the covariance matrix C_{HA} for the individuals in AD+ group. The normalized residual vector is denoted by r̅_{AD+}. For every individual, the mean of the absolute value of the inner product |<r̅_{AD+}, νᵢ>| (where vᵢ is the i-th eigenvector of C_{HA} was evaluated for the 100 VNN models.

Note that the VNNs were trained as described in Section 3.1 and hence, their ability to exploit the eigenvectors of the covariance matrix in a specific manner was learned when trained to predict chronological age for a healthy population. Hence, to gauge whether the anatomical interpretability was contingent on the learned ability of VNNs to exploit the eigenvectors of the covariance matrix from the procedure in Section 3.1, we also derived the anatomical interpretability for randomly initialized VNNs.

### 3.3 Individual-Level Brain Age Prediction

Finally, a scalar estimate for the brain age was obtained from the VNN regression output through a procedure consistent with the existing studies in this domain. Note that 100 VNNs provide 100 estimates ŷ of the chronological age for each subject. For simplicity, we consider ŷ to be the mean of these estimates. A systemic bias in the gap between ŷ and y may potentially exist when the correlation between ŷ and y is smaller than 1. Such a bias can confound the interpretations of brain age due to underestimation for older individuals and overestimation for younger individuals. Therefore, to correct for this age-induced bias in ŷ-y, we adopted a linear regression model-based approach [45, 46]. Specifically, the following bias correction steps were applied on the VNN estimated age y to obtain the brain age ŷ_{B} for an individual with chronological age y:

Step 1. Fit a regression model for the HC group to determine scalars a and β in the following model:

$$\hat{y} - y = α y + β . \qquad ( 8 )$$

Step 2. Evaluate brain age ŷ_{B} as follows:

$${\hat{y}}_{B} = \hat{y} - ( α y + β ) . \qquad ( 9 )$$

The gap between ŷ_{B} and y is the Δ-Age and is defined below. For an individual with cortical thickness x and chronological age y, the brain age gap Δ-Age is formally defined as

$$Δ - Age {=}^{Δ} {\hat{y}}_{B} - y , \qquad ( 10 )$$

where ŷ_{B} is determined from the VNN age estimate ŷ and y according to steps in (8) and (9). The age-bias correction in (8) and (9) was performed only for the HC group to account for bias in the VNN estimates due to healthy aging, and then applied to the AD+ group. Further, the distributions of Δ-Age were obtained for all individuals in HC and AD+ groups.

Δ-Age for AD+ group was expected to be elevated as compared to HC group as a consequence of elevated regional residuals derived from the VNN model. To elucidate this, consider a toy example with two individuals of the same chronological age y, with one belonging to the AD+ group and another to the HC group. Equation (9) suggests that their corresponding VNN outputs (denoted by ŷ_{AD+} for individual in the AD+ group and ŷ_{HC} for individual in the HC group) are corrected for age-bias using the same term αy+β. Hence, Δ-Age for the individual in the AD+ group will be elevated with respect to that from the HC group only if the VNN prediction ŷ_{AD+} is elevated with respect to ŷ_{HC}. Since the VNN predictions ŷ_{AD+} and ŷ_{HC} are proportional to the unweighted aggregations of the estimates at the regional level [see (4) and (5), larger ŷ_{AD+} with respect to ŷ_{HC} can be a direct consequence of a subset of regional residuals [see (7)] being robustly elevated in AD+ group with respect to HC group across the 100 VNN models. When the individuals in this example have different chronological age, the age-bias correction is expected to remove any variance due to chronological age in Δ-Age. We also verified that the differences in Δ-Age for AD+ and HC group were not driven by age or sex differences via ANCOVA with age and sex as covariates.

### 4. Results

### 4.1 Chronological Age Prediction for the HC Group

The performance achieved by the VNNs on the chronological age prediction task for the HC group has been summarized over the 100 nominal VNN models. VNNs achieved a mean absolute error (MAE) of 5.82±0.13 years and Pearson's correlation of 0.51±0.078 between the chronological age estimates and the ground truth on the test set. Moreover, on the complete dataset, the MAE was 5.44±0.18 years and Pearson's correlation was 0.47±0.074. Thus, the trained VNNs were not overfit on the training set.

Next, for every individual in the HC group, we evaluated the mean of the inner products (also equivalently referred to as dot product) between the vectors of contributions of every brain region [p in (5)] and the eigenvectors of the anatomical covariance matrix for all 100 VNN models. The strongest alignment was present between the first eigenvector of the anatomical covariance matrix (i.e., the eigenvector associated with the largest eigenvalue) and the vectors of regional contributions to the VNN output (0.987±0.0005 across the HC group), with relatively smaller associations for second (0.051±0.003), third (0.075±0.004), and fourth (0.094±0.003) eigenvectors. Additional details are included in Appendix F. Thus, the VNNs exploited the eigenvectors of the anatomical covariance matrix to achieve the learning performance in this task. The first eigenvector of the anatomical covariance matrix predominantly included bilateral anatomic brain regions in the parahippocampal gyrus, precuneus, inferior medial temporal gyrus, and precentral gyrus.

We remark that several existing studies on brain age prediction have utilized deep learning and other approaches to report better MAE on their respective healthy populations [20-22, 47]. In contrast, our contribution here is conceptual, where we have explored the properties of VNNs when they are trained to predict the chronological age for the HC group. Subsequently, our primary focus in the context of brain age is on demonstrating the anatomical interpretability offered by VNNs and relevance of eigenvectors of the anatomical covariance matrix. Thus, we further provide the insights that have not been explored (or are infeasible to obtain) for most existing brain age evaluation frameworks based on less transparent deep learning models.

4.2 Analyses of Regional Residuals Derived from VNNs Revealed Regions Characteristic of AD

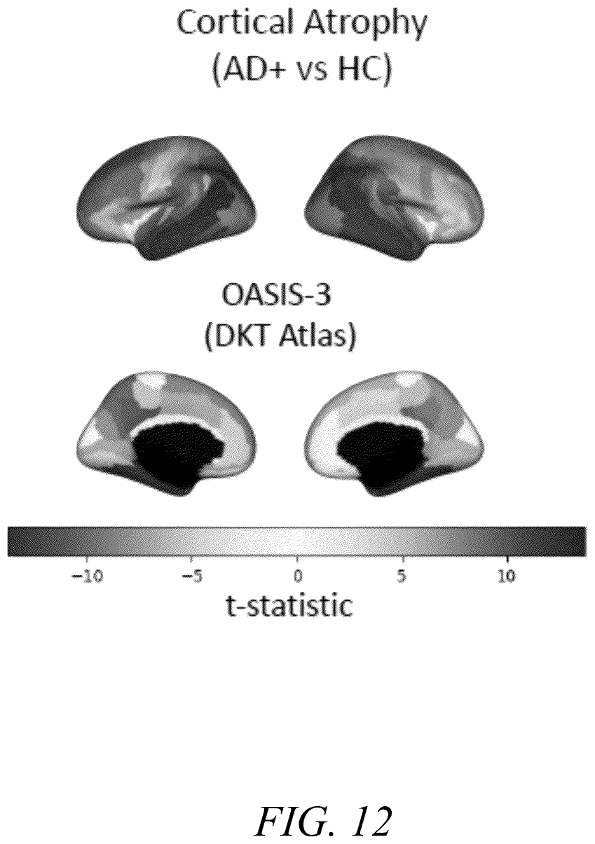

FIG. 3, panel a displays the robustness (determined via analyses of 100 VNN models) for various brain regions being associated with significantly larger residual elements for the AD+ group than the HC group. The most significant regions with elevated regional residuals in AD+ with respect to HC were concentrated in bilateral inferior parietal, temporal, entorhinal, parahippocampal, subcallosal, and precentral regions. All these brain regions, except for precentral and subcallosal, mirrored the cortical atrophy (FIG. 12), and these regions are known to be highly interconnected with hippocampus [48]. Hence, brain regions characteristic of AD had significant differences in regional residual distributions for AD+ group as compared to HC group.

Although the results in FIG. 3, panel a provided a meaningful regional profile for AD+ group, we further performed exploratory analysis to check whether the regional residuals had any clinical significance. To this end, we evaluated the Pearson's correlations between CDR sum of boxes and the regional residuals derived from final layer VNN outputs for the AD+ group for all 100 VNN models. Interestingly, the brain regions with the largest correlations with the CDR sum of boxes scores in FIG. 3, panel c were concentrated in the parahippocampal, medial temporal lobe, and temporal pole regions (also isolated in FIG. 3, panel a). This observation provides the evidence that the regional residuals for the AD+ group that led to the result in FIG. 3, panel a could predict dementia severity.

4.3 Δ-Age was Elevated in AD+ Group and Correlated with CDR

We evaluated the Δ-Age for HC and AD+ groups according to the procedure specified in Section 3.3. We also investigated the Pearson's correlation between Δ-Age and CDR sum of boxes scores in AD+ group. FIG. 3, panel b illustrates the distributions for Δ-Age for HC and AD+ groups (Δ-Age for HC: 0±2.83 years, Δ-Age for AD+: 3.54±4.49 years). The difference in Δ-Age for AD+ and HC groups was significant (Cohen's d=0.942, ANCOVA with age and sex as covariates: p-value <10⁻²⁰, partial η² 0.158). Also, age and sex were not significant in ANCOVA (p-value >0.4 for both). Hence, the group difference in Δ-Age for the two groups was not driven by the difference in the distributions of their chronological age. FIG. 3, panel d plots CDR sum of boxes scores versus Δ-Age for the AD+ group. Pearson's correlation between CDR sum of boxes score and Δ-Age was 0.474 (p-value=2.88×10⁻¹²), thus, implying that the Δ-Age evaluated for AD+ group captured information about dementia severity. Hence, as expected, the Δ-Age for AD+ was likely to be larger with an increase in CDR sum of boxes scores. For instance, the mean Δ-Age for individuals with CDR sum of boxes greater than 4 was 6.04 years, and for CDR sum of boxes ≤4 was 2.42 years.

Given that the age-bias correction procedure is a linear transformation of VNN outputs, it can readily be concluded that the statistical patterns for regional residuals in FIG. 3, panel a and FIG. 3, panel c lead to elevated Δ-Age and correlation between Δ-Age and CDR sum of boxes scores. Therefore, our framework provides a feasible way to characterize accelerated biological aging in AD+ group with a regional profile. Additional figures and details pertaining to VNN outputs and brain age before and after the age-bias correction was applied are described in Appendix I.

Regional residuals derived from VNNs were correlated with eigenvectors of the anatomical covariance matrix

FIG. 4, panel a plots the mean inner product |<r̅_{AD+}, νᵢ>| for eigenvectors associated with 50 largest eigenvalues of C_{HA}. The three largest mean correlations with the regional residuals in AD+ group were observed for the third eigenvector of C_{HA}(|<r̅_{AD+}, ν₃>|=0.645±0.016, fourth eigenvector (|<r̅_{AD+}, ν₄>|=0.305±0.02), and the first eigenvector (|<r̅_{AD+}, ν₁>|=0.299±0.001). These eigenvectors are plotted on a brain template in the expanded FIG. 4, Panel b. Inspection of the first, third, and fourth eigenvectors of C_{HA} suggested that subcallosal, entorhinal, parahippocampal and temporal pole regions had the most dominant weights (in terms of magnitude) in these eigenvectors.

### 4.5 Anatomical Interpretability was Diminished for Randomly Initialized VNNs.

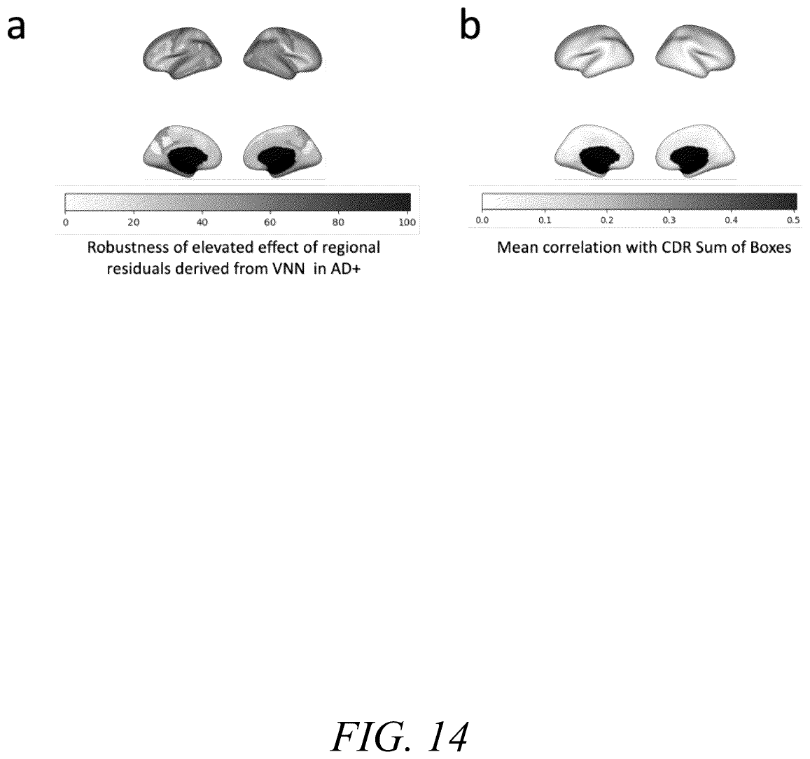

Finally, we leveraged 100 VNNs that were randomly initialized (i.e., not trained whatsoever) to evaluate the regional profiles for brain regions that exhibited elevated regional residuals for AD+ group with respect to HC group in OASIS-3. These VNNs had the same architecture as the VNNs that were trained on OASIS-3. FIG. 14, panel a shows that the robustness of the regional residuals being elevated for AD+ group with respect to HC group was severely diminished as compared to the parallel results in FIG. 3, Panel a. Similarly, the correlation between the regional residual derived using randomly initialized VNNs and CDR scores was highly inconsistent (FIG. 14, Panel b) as compared to parallel results in FIG. 3, Panel c. These findings suggested that the ability of VNNs to exploit the covariance matrix (learned when trained to predict chronological age of the HC group) was instrumental to the results derived in FIG. 3.

### Additional Experiments

Cross-validation: The results derived in FIG. 3, Panel a and FIG. 3, Panel b were cross-validated on the ADNI-1 dataset (see Appendix H for details).

Stability to perturbations in C_{HA}: As a consequence of the stability of VNNs, we observed that the regional profile for Δ-Age in FIG. 3, panel a was stable even when the covariance matrix C_{HA} was estimated by a variable composition of individuals from the HC and AD+ group (Appendix L). Anatomical covariance matrix and brain age: Use of anatomical covariance matrix derived from only HC group provides results consistent with FIG. 3, albeit with a slightly smaller group difference between the Δ-Age distributions for HC and AD+ groups. See Appendix L for details.

### Discussion

Our study has proposed a methodologically transparent framework for brain age prediction using VNNs. In contrast to existing studies that primarily focus on the performance of the model in predicting the chronological age of a healthy population, we have focused on the properties that VNNs gained when trained for this task. In particular, the VNNs achieve learning by transforming the input data according to the principal components or the eigenvectors of the covariance matrix estimated from the data. Hence, training the VNNs to predict chronological age using cortical thickness features from a healthy population had enabled them to exploit the eigenvectors of the anatomical covariance matrix in a specific manner. Further, the anatomical interpretability associated with elevated Δ-Age in Alzheimer's disease was derived by the statistical analyses of the features extracted at the final layer of the VNNs and projecting the results of these analyses on the brain surface. Thus, the anatomical interpretability offered by VNNs is an inherent feature of VNNs and fundamentally distinct from the state-of-the-art model agnostic explainability approaches in the context of brain age (such as SHAP, LIME, saliency maps etc., that provide importance weights to individual input features).

The lack of explainability in the machine learning models deployed in brain age prediction frameworks may be a fundamental reason behind exclusive focus on the performance-driven approaches in this domain thus far. VNNs are inherently explainable models, as their inference outcomes can be tied with the eigenvectors of the covariance matrix. Hence, the anatomical interpretability offered by VNNs to Δ-Age could be explained by their ability to exploit specific eigenvectors of the anatomical covariance matrix. This observation is highly relevant, as the quality of prediction on the chronological prediction for healthy population by itself may not be a complete determinant of the quality of brain age prediction in neurodegeneration. Furthermore, the role of the age-bias correction step in the VNN-based brain age prediction framework was restricted to projecting the VNN outputs onto a space where one could infer accelerated biological aging with respect to the chronological age from a layman's perspective.

By associating Δ-Age with a regional profile, VNNs also provide a feasible tool to distinguish pathologies if the distributions of Δ-Age for them are overlapping. A larger focus is needed on principled statistical approaches for brain age prediction that can capture the factors that lead to accelerated aging. Locally interpretable and theoretically grounded deep learning models such as VNNs can provide a feasible, promising future direction to build statistically and conceptually legitimate brain age prediction models in broader contexts. Incorporating other modalities of neuroimaging or alternative metrics of aging other than chronological age (such as DNA methylation [49]) provide promising future directions that can help improve our understanding of aging.

The disclosure of each of the following references is incorporated herein by reference in its entirety.

### REFERENCES

[1] Carlos López-Otin et al. The hallmarks of aging. Cell, 153(6):1194-1217, 2013.
[2] Giovanni B Frisoni, Nick C Fox, Clifford R Jack, Philip Scheltens, and Paul M Thompson. The clinical use of structural MRI in Alzheimer disease. Nature Reviews Neurology, 6(2):67-77, 2010.
[3] J Jean Chen. Functional MRI of brain physiology in aging and neurodegenerative diseases. Neuroimage, 187:209-225, 2019.
[4] Ruth Peters. Ageing and the brain. Postgraduate medical journal, 82(964):84-88, 2006.
[5] James H Cole and Katja Franke. Predicting age using neuroimaging: Innovative brain ageing biomarkers. Trends in Neurosciences, 40(12):681-690, 2017.
[6] James H Cole, Stuart J Ritchie, Mark E Bastin, Valdés Hernández, S Muñoz Maniega, Natalie Royle, Janie Corley, Alison Pattie, Sarah E Harris, Qian Zhang, et al. Brain age predicts mortality. Molecular Psychiatry, 23(5):1385-1392, 2018.
[7] Lea Baecker, Rafael Garcia-Dias, Sandra Vieira, Cristina Scarpazza, and Andrea Mechelli. Machine learning for brain age prediction: Introduction to methods and clinical applications. EBioMedicine, 72:103600, 2021.
[8] Lea Baecker, Jessica Dafflon, Pedro F Da Costa, Rafael Garcia-Dias, Sandra Vieira, Cristina Scarpazza, Vince D Calhoun, Joao R Sato, Andrea Mechelli, and Walter H L Pinaya. Brain age prediction: A comparison between machine learning models using region- and voxel-based morphometric data. Human Brain Mapping, 42(8):2332-2346, 2021.
[9] Iman Beheshti, M A Ganaie, Vardhan Paliwal, Aryan Rastogi, Imran Razzak, and Muhammad Tanveer. Predicting brain age using machine learning algorithms: A comprehensive evaluation. IEEE Journal of Biomedical and Health Informatics, 26(4):1432-1440, 2021.
[10] Oscar Pina, Irene Cumplido-Mayoral, Raffaele Cacciaglia, José Maria González-de Echávarri, Juan Domingo Gispert, and Verónica Vilaplana. Structural networks for brain age prediction. In International Conference on Medical Imaging with Deep Learning, pages 944-960. PMLR, 2022.
[11] Katja Franke and Christian Gaser. Ten years of brain age as a neuroimaging biomarker of brain aging: What insights have we gained?Frontiers in neurology, page 789, 2019.
[12] Katja Franke and Christian Gaser. Longitudinal changes in individual brain age in healthy aging, mild cognitive impairment, and Alzheimer's disease. GeroPsych: The Journal of Gerontopsychology and Geriatric Psychiatry, 25(4):235, 2012.
[13] M Habes, D Janowitz, G Erus, J B Toledo, S M Resnick, J Doshi, S Van der Auwera, K Wittfeld, K Hegenscheid, N Hosten, et al. Advanced brain aging: Relationship with epidemiologic and genetic risk factors, and overlap with Alzheimer disease atrophy patterns. Translational Psychiatry, 6(4):e775-e775, 2016.[14] Mariona Jové, Manuel Portero-Otín, Alba Naudí, Isidre Ferrer, and Reinald Pamplona. Metabolomics of human brain aging and age-related neurodegenerative diseases. Journal of Neuropathology & Experimental Neurology, 73(7):640-657, 2014.
[15] Tomas Hajek, Katja Franke, Marian Kolenic, Jana Capkova, Martin Matejka, Lukas Propper, Rudolf Uher, Pavla Stopkova, Tomas Novak, Tomas Paus, et al. Brain age in early stages of bipolar disorders or schizophrenia. Schizophrenia Bulletin, 45(1):190-198, 2019.
[16] Scott M McGinnis, Michael Brickhouse, Belen Pascual, and Bradford C Dickerson. Age-related changes in the thickness of cortical zones in humans. Brain Topography, 24(3):279-291, 2011.
[17] Manja Lehmann, Sebastian J Crutch, Gerard R Ridgway, Basil H Ridha, Josephine Barnes, Elizabeth K Warrington, Martin N Rossor, and Nick C Fox. Cortical thickness and voxel-based morphometry in posterior cortical atrophy and typical Alzheimer's disease. Neurobiology of aging, 32(8):1466-1476, 2011.
[18] Mert R Sabuncu, Rahul S Desikan, Jorge Sepulcre, Boon Thye T Yeo, Hesheng Liu, Nicholas J Schmansky, Martin Reuter, Michael W Weiner, Randy L Buckner, Reisa A Sperling, et al. The dynamics of cortical and hippocampal atrophy in Alzheimer disease. Archives of neurology, 68 (8):1040-1048, 2011.
[19] Alan C Evans. Networks of anatomical covariance. Neuroimage, 80:489-504, 2013.
[20] Chenzhong Yin, Phoebe Imms, Mingxi Cheng, Anar Amgalan, Nahian F Chowdhury, Roy J Massett, Nikhil N Chaudhari, Xinghe Chen, Paul M Thompson, Paul Bogdan, et al. Anatomically interpretable deep learning of brain age captures domain-specific cognitive impairment. Proceedings of the National Academy of Sciences, 120(2):e2214634120, 2023.
[21] Vishnu M Bashyam, Guray Erus, Jimit Doshi, Mohamad Habes, Ilya M Nasrallah, Monica Truelove-Hill, Dhivya Srinivasan, Liz Mamourian, Raymond Pomponio, Yong Fan, et al. MRI signatures of brain age and disease over the lifespan based on a deep brain network and 14 468 individuals worldwide. Brain, 143(7):2312-2324, 2020.
[22] Baptiste Couvy-Duchesne, Johann Faouzi, Benoît Martin, Elina Thibeau-Sutre, Adam Wild, Manon Ansart, Stanley Durrleman, Didier Dormont, Ninon Burgos, and Olivier Colliot. Ensemble learning of convolutional neural network, support vector machine, and best linear unbiased predictor for brain age prediction: Aramis contribution to the predictive analytics competition 2019 challenge. Frontiers in Psychiatry, 11:593336, 2020.
[23] Ellyn R Butler, Andrew Chen, Rabie Ramadan, Trang T Le, Kosha Ruparel, Tyler M Moore, Theodore D Satterthwaite, Fengqing Zhang, Haochang Shou, Ruben C Gur, et al. Pitfalls in brain age analyses. Technical report, Wiley Online Library, 2021.
[24] Angela Lombardi, Domenico Diacono, Nicola Amoroso, Alfonso Monaco, João Manuel R S Tavares, Roberto Bellotti, and Sabina Tangaro. Explainable deep learning for personalized age prediction with brain morphology. Frontiers in neuroscience, 15:578, 2021.
[25] Jeyeon Lee, Brian J Burkett, Hoon-Ki Min, Matthew L Senjem, Emily S Lundt, Hugo Botha, Jonathan Graff-Radford, Leland R Barnard, Jeffrey L Gunter, Christopher G Schwarz, et al. Deep learning-based brain age prediction in normal aging and dementia. Nature Aging, 2(5): 412-424, 2022.
[26] Simon M Hofmann, Frauke Beyer, Sebastian Lapuschkin, Ole Goltermann, Markus Loeffler, Klaus-Robert Müller, Arno Villringer, Wojciech Samek, and A Veronica Witte. Towards the interpretability of deep learning models for multi-modal neuroimaging: Finding structural changes of the ageing brain. NeuroImage, 261:119504, 2022.
[27] Ann-Kathrin Dombrowski, Maximillian Alber, Christopher Anders, Marcel Ackermann, Klaus-Robert Müller, and Pan Kessel. Explanations can be manipulated and geometry is to blame. Advances in neural information processing systems, 32, 2019.
[28] Julius Adebayo, Justin Gilmer, Michael Muelly, Ian Goodfellow, Moritz Hardt, and Been Kim. Sanity checks for saliency maps. Advances in neural information processing systems, 31, 2018.
[29] Emily Black, Manish Raghavan, and Solon Barocas. Model multiplicity: Opportunities, concerns, and solutions. In Proceedings of the 2022 ACM Conference on Fairness, Accountability, and Transparency, pages 850-863, 2022.
[30] Robert J Jirsaraie, Aaron J Gorelik, Martins M Gatavins, Denis A Engemann, Ryan Bogdan, Deanna M Barch, and Aristeidis Sotiras. A systematic review of multimodal brain age studies: Uncovering a divergence between model accuracy and utility. Patterns, 4(4), 2023.
[31] Saurabh Sihag, Gonzalo Mateos, Corey McMillan, and Alejandro Ribeiro. coVariance neural networks. In Proc. Conference on Neural Information Processing Systems, Nov. 2022.
[32] Hao Yuan, Haiyang Yu, Shurui Gui, and Shuiwang Ji. Explainability in graph neural networks: A taxonomic survey. IEEE transactions on pattern analysis and machine intelligence, 45(5): 5782-5799, 2022.
[33] Pamela J LaMontagne, Tammie L S Benzinger, John C Morris, Sarah Keefe, Russ Hornbeck, Chengjie Xiong, Elizabeth Grant, Jason Hassenstab, Krista Moulder, Andrei G Vlassenko, et al. OASIS-3: longitudinal neuroimaging, clinical, and cognitive dataset for normal aging and Alzheimer disease. MedRxiv, 2019.
[34] Bradley T Wyman, Danielle J Harvey, Karen Crawford, Matt A Bernstein, Owen Carmichael, Patricia E Cole, Paul K Crane, Charles DeCarli, Nick C Fox, Jeffrey L Gunter, et al. Standardization of analysis sets for reporting results from adni mire data. Alzheimer's & Dementia, 9(3): 332-337, 2013.
[35] Saurabh Sihag, Gonzalo Mateos, Corey McMillan, and Alejandro Ribeiro. Predicting brain age using transferable coVariance neural networks. In Proc. IEEE International Conference on Acoustics, Speech, and Signal Processing, Jun. 2023.
[36] Fernando Gama, Elvin Isufi, Geert Leus, and Alejandro Ribeiro. Graphs, convolutions, and neural networks: From graph filters to graph neural networks. IEEE Signal Processing Magazine, 37(6):128-138, 2020.
[37] Antonio Ortega, Pascal Frossard, Jelena Kovacevic, Jose M F Moura, and Pierre Vandergheynst. Graph signal processing: Overview, challenges, and applications. Proceedings of the IEEE, 106 (5):808-828, 2018.
[38] Fernando Gama, Joan Bruna, and Alejandro Ribeiro. Stability properties of graph neural networks. IEEE Transactions on Signal Processing, 68:5680-5695, 2020.
[39] Si Zhang, Hanghang Tong, Jiejun Xu, and Ross Maciejewski. Graph convolutional networks: A comprehensive review. Computational Social Networks, 6(1):1-23, 2019.
[40] David I Shuman, Sunil K Narang, Pascal Frossard, Antonio Ortega, and Pierre Vandergheynst. The emerging field of signal processing on graphs: Extending high-dimensional data analysis to networks and other irregular domains. IEEE Signal Processing Magazine, 30(3):83-98, 2013.
[41] Christophe Destrieux, Bruce Fischl, Anders Dale, and Eric Halgren. Automatic parcellation of human cortical gyri and sulci using standard anatomical nomenclature. Neuroimage, 53(1): 1-15, 2010.
[42] Sid E O'Bryant, Stephen C Waring, C Munro Cullum, James Hall, Laura Lacritz, Paul J Mass-man, Philip J Lupo, Joan S Reisch, Rachelle Doody, Texas Alzheimer's Research Consortium, et al. Staging dementia using clinical dementia rating scale sum of boxes scores: A Texas Alzheimer's research consortium study. Archives of Neurology, 65(8):1091-1095, 2008.
[43] John C Morris. The clinical dementia rating (cdr): current version and scoring rules. Neurology, 1993.
[44] Takuya Akiba, Shotaro Sano, Toshihiko Yanase, Takeru Ohta, and Masanori Koyama. Optuna: A next-generation hyperparameter optimization framework. In Proceedings of the 25th ACM SIGKDD International Conference on Knowledge Discovery & Data Mining, pages 2623-2631, 2019.
[45] Iman Beheshti, Scott Nugent, Olivier Potvin, and Simon Duchesne. Bias-adjustment in neuroimaging-based brain age frameworks: A robust scheme. NeuroImage: Clinical, 24: 102063, 2019.
[46] Ann-Marie G de Lange and James H Cole. Commentary: Correction procedures in brain-age prediction. NeuroImage: Clinical, 26, 2020.
[47] Franziskus Liem, Gasl Varoquaux, Jana Kynast, Frauke Beyer, Shahrzad Kharabian Masouleh, Julia M Huntenburg, Leonie Lampe, Mehdi Rahim, Alexandre Abraham, R Cameron Craddock, et al. Predicting brain-age from multimodal imaging data captures cognitive impairment. Neuroimage, 148:179-188, 2017.
[48] Olof Lindberg, Eric Westman, Sari Karlsson, Per Östberg, Leif A Svensson, Andrew Simmons, and Lars-Olof Wahlund. Is the subcallosal medial prefrontal cortex a common site of atrophy in Alzheimer's disease and frontotemporal lobar degeneration?Frontiers in Aging Neuroscience, 4: 32, 2012.
[49] Steve Horvath. DNA methylation age of human tissues and cell types. Genome biology, 14(10): 1-20, 2013.
[50] Lisa Ronan, Aaron F Alexander-Bloch, Konrad Wagstyl, Sadaf Farooqi, Carol Brayne, Lorraine K Tyler, Paul C Fletcher, et al. Obesity associated with increased brain age from midlife. Neurobiology of aging, 47:63-70, 2016.
[51] Klara Mareckova, Radek Marecek, Lenka Andryskova, Milan Brazdil, and Yuliya S Nikolova. Maternal depressive symptoms during pregnancy and brain age in young adult offspring: findings from a prenatal birth cohort. Cerebral Cortex, 30(7):3991-3999, 2020.
[52] Lars T Westlye, Ivar Reinvang, Helge Rootwelt, and Thomas Espeseth. Effects of apoe on brain white matter microstructure in healthy adults. Neurology, 79(19):1961-1969, 2012.
[53] Joan Bruna, Wojciech Zaremba, Arthur Szlam, and Yann LeCun. Spectral networks and locally connected networks on graphs. arXiv preprint arXiv:1312.6203, 2013.
[54] David K Hammond, Pierre Vandergheynst, and Remi Gribonval. Wavelets on graphs via spectral graph theory. Applied and Computational Harmonic Analysis, 30(2):129-150, 2011.
[55] James Atwood and Don Towsley. Diffusion-convolutional neural networks. Advances in neural information processing systems, 29, 2016.
[56] Saurabh Verma and Zhi-Li Zhang. Stability and generalization of graph convolutional neural networks. In Proceedings of the 25th ACM SIGKDD International Conference on Knowledge Discovery & Data Mining, pages 1539-1548, 2019.
[57] Nicolas Keriven, Alberto Bietti, and Samuel Vaiter. Convergence and stability of graph convolutional networks on large random graphs. Advances in Neural Information Processing Systems, 33:21512-21523, 2020.
[58] Luana Ruiz, Luiz Chamon, and Alejandro Ribeiro. Graphon neural networks and the transferability of graph neural networks. Advances in Neural Information Processing Systems, 33: 1702-1712, 2020.
[59] Hiroshi Murase and Shree K. Nayar. Illumination planning for object recognition using parametric eigenspaces. IEEE Transactions on Pattern Analysis and Machine Intelligence, 16 (12):1219-1227, 1994.
[60] Fernando De la Torre and Michael J Black. Robust principal component analysis for computer vision. In Proceedings Eighth IEEE International Conference on Computer Vision. ICCV 2001, volume 1, pages 362-369. IEEE, 2001.
[61] DB Stephenson. Correlation of spatial climate/weather maps and the advantages of using the mahalanobis metric in predictions. Tellus A, 49(5):513-527, 1997.
[62] Hu Shao, William H K Lam, Agachai Sumalee, Anthony Chen, and Martin L Hazelton. Estimation of mean and covariance of peak hour origin-destination demands from day-to-day traffic counts. Transportation Research Part B: Methodological, 68:52-75, 2014.
[63] Mohd Nazri Ismail, Abdulaziz Aborujilah, Shahrulniza Musa, and AAmir Shahzad. Detecting flooding based dos attack in cloud computing environment using covariance matrix approach. In Proceedings of the 7th international conference on ubiquitous information management and communication, pages 1-6, 2013.
[64] Jaykumar Kakkad, Jaspal Jannu, Kartik Sharma, Charu Aggarwal, and Sourav Medya. A survey on explainability of graph neural networks. arXiv preprint arXiv:2306.01958, 2023.
[65] Phillip E Pope, Soheil Kolouri, Mohammad Rostami, Charles E Martin, and Heiko Hoffmann. Explainability methods for graph convolutional neural networks. In Proceedings of the IEEE/CVF conference on computer vision and pattern recognition, pages 10772-10781, 2019.
[66] Zhitao Ying, Dylan Bourgeois, Jiaxuan You, Marinka Zitnik, and Jure Leskovec. Gnnexplainer: Generating explanations for graph neural networks. Advances in neural information processing systems, 32, 2019.
[67] Qiang Huang, Makoto Yamada, Yuan Tian, Dinesh Singh, and Yi Chang. Graphlime: Local interpretable model explanations for graph neural networks. IEEE Transactions on Knowledge and Data Engineering, 2022.
[68] Hao Yuan, Jiliang Tang, Xia Hu, and Shuiwang Ji. Xgnn: Towards model-level explanations of graph neural networks. In Proceedings of the 26th ACM SIGKDD International Conference on Knowledge Discovery & Data Mining, pages 430-438, 2020.
[69] Hao Yuan, Haiyang Yu, Jie Wang, Kang Li, and Shuiwang Ji. On explainability of graph neural networks via subgraph explorations. In International conference on machine learning, pages 12241-12252. PMLR, 2021.
[70] Sebastian G Popescu, Ben Glocker, David J Sharp, and James H Cole. Local brain-age: a u-net model. Frontiers in Aging Neuroscience, 13:761954, 2021.
[71] Xinyang Feng, Zachary C Lipton, Jie Yang, Scott A Small, Frank A Provenzano, Alzheimer's Disease Neuroimaging Initiative, Frontotemporal Lobar Degeneration Neuroimaging Initiative, et al. Estimating brain age based on a uniform healthy population with deep learning and structural magnetic resonance imaging. Neurobiology of aging, 91:15-25, 2020.
[72] Xinyang Feng, Jie Yang, Zachary C Lipton, Scott A Small, Frank A Provenzano, Alzheimer's Disease Neuroimaging Initiative, et al. Deep learning on MRI affirms the prominence of the hippocampal formation in Alzheimer's disease classification. bioRxiv, page 456277, 2018.
[73] Achraf Essemlali, Etienne St-Onge, Maxime Descoteaux, and Pierre-Marc Jodoin. Understanding Alzheimer disease's structural connectivity through explainable AI. In Medical Imaging with Deep Learning, pages 217-229. PMLR, 2020.
[74] Helmet T Karim, Maria Ly, Gary Yu, Robert Krafty, Dana L Tudorascu, Howard J Aizenstein, and Carmen Andreescu. Aging faster: Worry and rumination in late life are associated with greater brain age. Neurobiology of Aging, 101:13-21, 2021.
[75] Adam Paszke, Sam Gross, Francisco Massa, Adam Lerer, James Bradbury, Gregory Chanan, Trevor Killeen, Zeming Lin, Natalia Gimelshein, Luca Antiga, et al. Pytorch: An imperative style, high-performance deep learning library. Advances in Neural Information Processing Systems, 32, 2019.
[76] Christian Gaser, Robert Dahnke, Paul M Thompson, Florian Kurth, Eileen Luders, and Alzheimer's Disease Neuroimaging Initiative. Cat-a computational anatomy toolbox for the analysis of structural mri data. biorxiv, pages 2022-06, 2022.
[77] Gary W van Hoesen, Jean C Augustinack, Jason Dierking, Sarah J Redman, and Ramasamy Thangavel. The parahippocampal gyrus in Alzheimer's disease: clinical and preclinical neuroanatomical correlates. Annals of the New York Academy of Sciences, 911(1):254-274, 2000.

### Appendix A: Data and Code Availability

The OASIS-3 dataset is publicly available and hosted on central.xnat.org. Access to OASIS-3 dataset may be requested through https://www.oasis-brains.org/. Supplementary files include (i) the VNN models trained to predict chronological age for HC group, (ii) code for demonstrations of evaluating regional profiles corresponding to elevated regional residuals in AD+ group, and correlations between the eigenvectors of the anatomical covariance and VNN outputs using a small subset of z-score normalized cortical thickness data, (iii) the regional residuals derived from different VNN models for the OASIS-3 dataset that lead to results in FIG. 3, and (iv) the code for brain age evaluation on OASIS-3 dataset. All material is also available online at https://github.com/sihagsNNN_Brain_Age.

### Appendix B: Relevant Literature

Graph convolutional networks. GCNs typically rely on an information aggregation procedure (referred to as graph convolutions) over a graph structure for data processing. Several implementation strategies for graph convolution operations have been proposed in the literature, including spectral convolutions [53], Chebyshev polynomials [54], ordinary polynomials [36], and diffusion based representations [55]. GCNs admit the properties of stability to topological perturbations and transferability across graphs of different sizes in various settings [38, 56-58], which makes them a well-motivated data analysis tool for graph-structured data.

In [31], coVariance neural networks (VNN) were proposed as GCNs with sample covariance matrices as graph and polynomial graph filters as convolution operation. Covariance matrices and principal component analysis (PCA) form the two cornerstones of non-parametric analyses in real world applications that have spatially distributed, multi-variate data acquisition protocols, including neuroimaging [19], computer vision [59], [60], weather modeling [61], traffic flow analysis [62], and cloud computing [63].

Explainability in GNNs. We refer the reader to [32, 64] for a detailed review on explainability in GNNs. Here, we adopt the taxonomy from [32], which categorizes the recent efforts to add explainability to GNN models into two categories: instance-level methods and model-level methods for explainability. Instance-level methods for explainability are extensions of the standard model-agnostic methods for wider categories of deep learning models to GNNs, and aim to identify features most important to the inference outcome. Examples of techniques used to determine the importance of features include gradient-based methods [65], perturbation-based methods [66], and surrogate methods [67]. Such methods are, in principle, input-dependent as they provide explanations based on the given instance of the dataset. The robustness of the conclusions drawn from such methods to perturbations in the input and variations in the model training algorithms is an active area of research.

Model-level explainability has previously been studied in terms of graph topology in [68]. Further, importance of subgraphs to inference outcomes using Shapley [69] values was studied in. Since graph topology informs convolution operations in GNNs, it can provide a more generic explanation to GNNs than those that focus on individual features (such as nodes or edges of the graph). Since the convolution operation in VNNs is equivalent to manipulating the input data according to the eigenvectors of the covariance matrix [31], the eigenvectors of the covariance matrix are instrumental to explaining the inference by VNNs. Hence, we argue that VNNs can offer model-level explainability, similar in spirit as discussed in [32], where the explainability hinges on the eigenvectors of the covariance matrix.

Interpretable brain age prediction. Limited focus has been on comparable studies that associate brain age gaps with regional profiles [20, 70]. The study in [20] adopts a convolutional neural network approach to infer brain age from MRI images directly and assigns importance to brain regions in evaluating the brain age. In principle, the interpretability offered by VNNs in the context of brain age is similar, as we infer a regional profile for Δ-Age by isolating the brain regions that are contributors to the elevated Δ-Age in neurodegeneration. In addition, the regional profile identified by VNNs is correlated with specific eigenvectors or the principal components of the anatomical covariance matrix. Hence, the Δ-Age inferred by our framework is driven by the ability of a VNN to manipulate the input data according to certain principal components of the anatomical covariance matrix. Also, VNNs are significantly less complex deep learning models as compared to those studied in [20]. Our results demonstrate that the VNNs trained with less than 300 learnable parameters exhibit regional interpretability in the context of brain age in AD. In general, the regional expressivity offered by VNNs is in stark contrast to a multitude of existing relevant studies that rely on less transparent statistical approaches and further use post-hoc analyses (such as ablation analysis [71-73] or exploring correlations with region-specific markers [25] and psychiatric symptoms [47,74]) to assign interpretability to a scalar, elevated Δ-Age effect.

### Appendix C: An Abstract Overview of VNN-based Brain Age Prediction

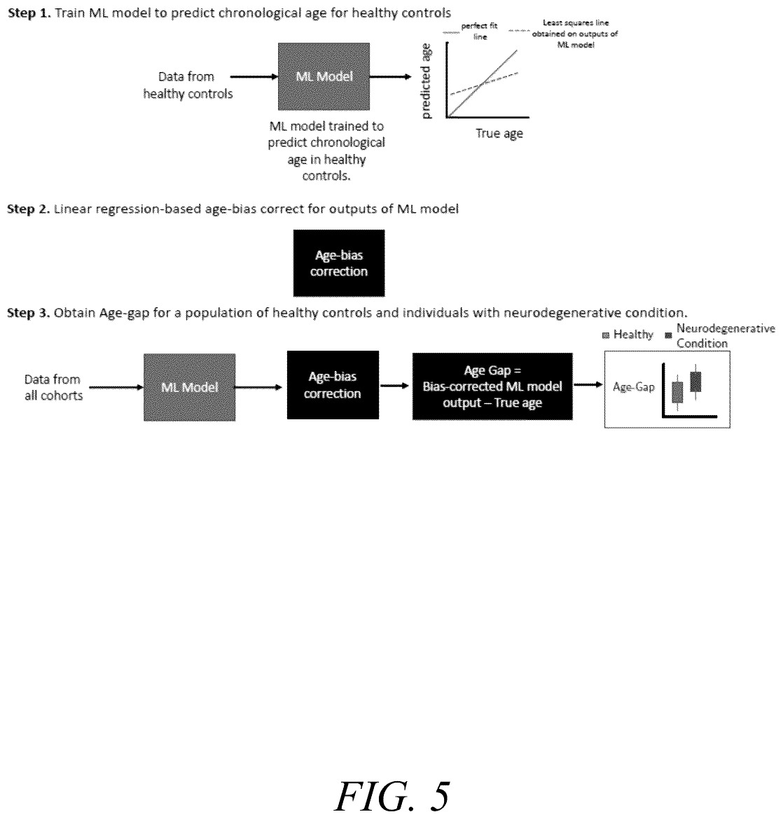

FIG. 5 provides an abstract overview of the general procedure of evaluating brain age using machine learning (ML) models. From FIG. 5, we note that if the ML model is a black box, it may be infeasible to capture the contributors to elevated age-gap in Step 3. Furthermore, in this context, it is also unclear whether age-bias correction step (Step 2) influences final Δ-Age prediction through some statistical artifact [23]. Hence, it can be desirable to minimize the role of age-bias correction in Δ-Age evaluation by selecting an ML model that achieves a near perfect fit on chronological age of healthy controls in Step 1. However, there is no guarantee that achieving an ‘perfect fit’ on true age of healthy controls will enable the ML model to capture the impact of neurodegeneration in individuals with neurodegeneration.

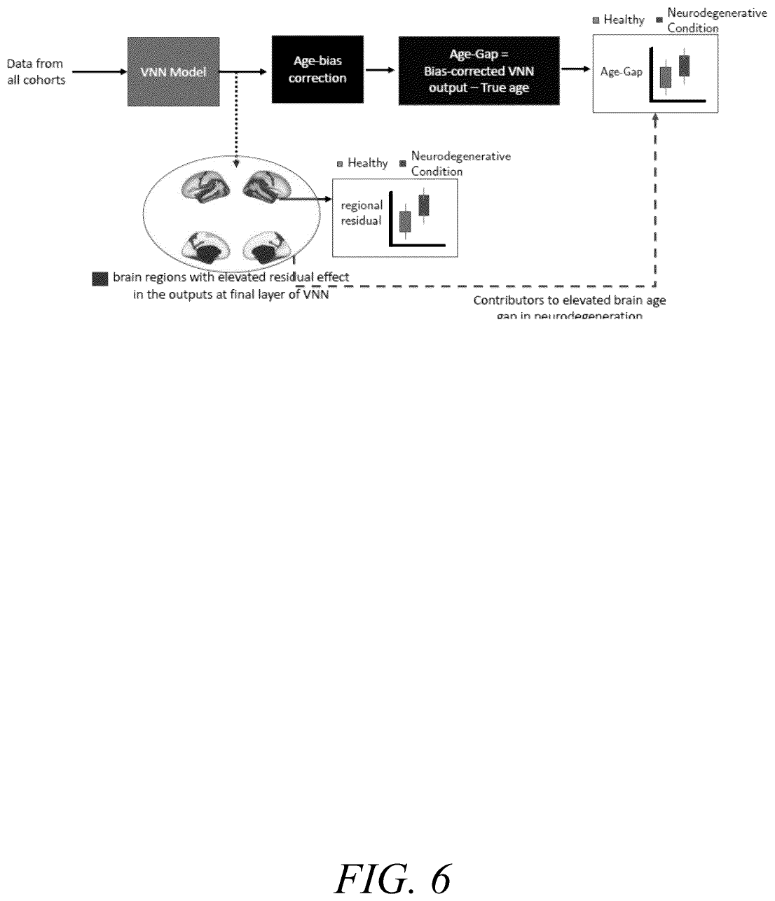

VNNs allow us to analyze the contribution of each feature (brain region) to the final output. Hence, by analyzing the elevations in contributions of different brain regions via studying group differences in regional residuals, we are able to characterize the brain regions that contribute to accelerated aging (or larger Δ-Age) in individuals with neurodegeneration (FIG. 6). Thus, we can verify that VNNs captured neurodegeneration-driven effects that eventually led to elevated Δ-Age for an individual. Our experiments show that VNNs do not obtain a perfect fit on chronological age of healthy individuals. Hence, age-bias correction is important to appropriately project the VNN model outputs via a linear model into an appropriate space such that a clinician can observe an elevated Δ-Age effect in individuals with neurodegenerative condition (AD in this paper). Based on these observations, we remark that VNNs provide an interpretable framework for brain age prediction.

### Appendix D: VNN Architecture

FIG. 7 illustrates the basics of the VNN architecture. Panel a illustrates that the coVariance filter H(C) is a polynomial in C and its application on input x. Panel b shows the construction of a coVariance perceptron based on coVariance filter H(C) and pointwise nonlinearity a. coVariance perceptron specifies one layer of VNN. Panel c shows a basic multi-layer VNN architecture formed by stacking three coVariance perceptrons.

Basics of VNN architecture. Panel a illustrates that the coVariance filter H(C) is a polynomial in C and its application on input x. Panel b shows the construction of a coVariance perceptron based on coVariance filter H(C) and pointwise nonlinearity a. coVariance perceptron specifies one layer of VNN. Panel c shows a basic multi-layer VNN architecture formed by stacking three coVariance perceptrons.

### Appendix E: VNN Training

We randomly split the HC group into an approximately 90/10 train/test split. Thus, the test set consisted of 61 healthy individuals. The sample covariance matrix was evaluated using all samples in the training set (n=550). Furthermore, this covariance matrix was normalized such that its maximum eigenvalue was 1. Cortical thickness data was z-score normalized across the training set and this normalization was applied to the test set. Next, the training set was randomly split internally, such that, the VNN was trained with respect to the mean squared error loss between the predicted age and the true age in n=489 samples of the HC group. The loss was optimized using batch stochastic gradient descent with Adam optimizer available in PyTorch library [75] for up to 100 epochs. The batch size was 78 (determined via ‘optuna’ package [44]). The VNN model with the best minimum mean squared error performance on the remaining 61 samples in the training set (which acted as a validation set) was included in the set of nominal models for this permutation of the training set. For each dataset, we trained and validated the VNN models over 100 permutations of the complete training set of n=550 samples for the HC group, thus, leading to 100 trained VNN models (also referred to as nominal models) per dataset.

### Appendix F: VNN Regression Model Outputs for HC Group in OASIS-3 are Correlated with the First Eigenvector of Anatomical Covariance Matrix

The study in [31] suggests that VNN based statistical inference draws conceptual similarities with PCA-driven analysis. Hence, we further investigated whether the regression performance achieved by VNNs in predicting the chronological age of HC group could be characterized by contributions of the eigenvectors of the anatomical covariance matrix. To avoid any selection bias, we report the results on the complete HC group, where the cortical thickness features were z-score normalized across the group such that the mean of cortical thickness for a brain region across the HC group was 0. Here, the notation C_{H} denotes the anatomical covariance matrix derived from the complete HC group.

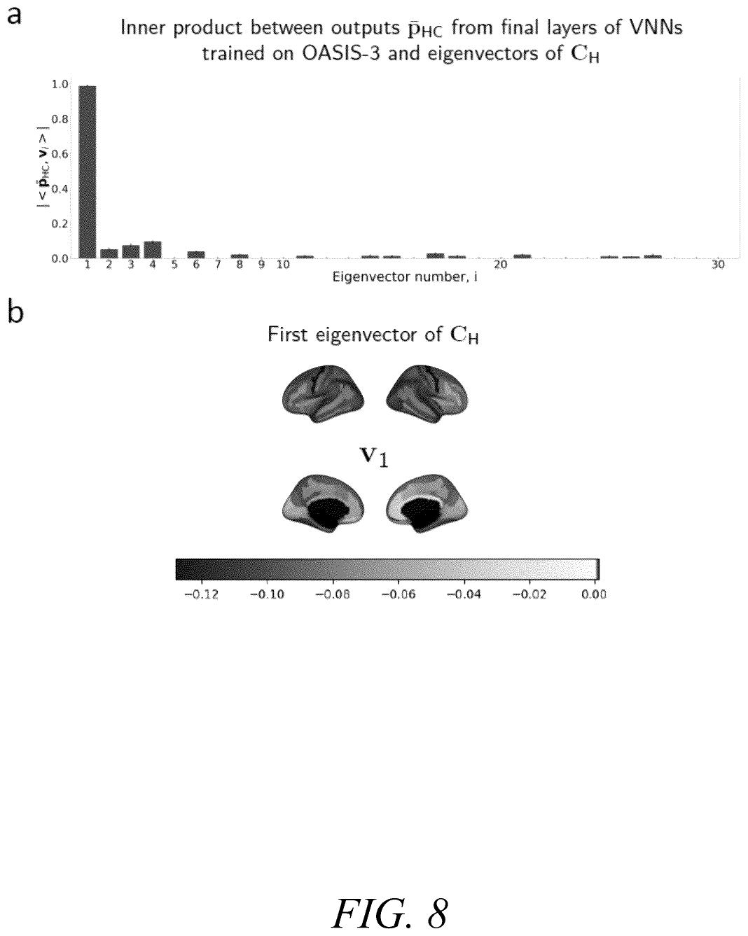

Recall that the final regression output by VNNs is formed by an unweighted average function as a readout function. Thus, we can equivalently represent the functionality of the readout as a simple aggregation of the contributions of different features or brain regions to the final estimate formed by the VNN (see [5]). Hence, for every individual, we evaluated the mean of the inner products (also equivalently referred to as dot product between vectors) between the vectors of contributions of every brain region with the eigenvector of the covariance matrix C_{H} for all 100 VNN models. Note that a vector of regional contributions was of the same length as the number of cortical thickness features (i.e., 148 for OASIS-3) and therefore, each element of this vector was associated with a distinct brain region. We use the notation P̅HC to represent the population of vectors obtained from the HC group. To evaluate the inner product, we used P̅HC, which was obtained from PHC after normalization (norm=1). We denote the population of inner products across the HC group in OASIS-3 by |<P̅_{HC}, νᵢ>| for an eigenvector vᵢ of C_{H}. Note that since all vectors in P̅_{HC} were normalized to have norm 1 and the eigenvectors of C_{H} were of length 1 by default, |<P̅_{HC} νᵢ>| represented the population of the cosine of the angles between the vectors in P_{HC} and eigenvectors vᵢ of C_{H} across the HC group in OASIS-3. FIG. 8, Panel a plots the mean of the inner products observed across the HC group for the first 30 eigenvectors of C_{H} for all 100 VNNs. FIG. 8, Panel b illustrates the projection of v₁ on a brain template.

### Appendix G: Illustration of Regional Residual Analysis from VNN Model Outputs

In this section, we demonstrate the regional analysis described in Section 3.2 for a VNN model that was trained to predict chronological age for HC group in OASIS-3 dataset. All mathematical notations referred to in this section are borrowed from Section 3.2. Note that no further training was performed for this VNN model to evaluate brain age or regional residuals.

The covariance matrix in this VNN model was replaced with C_{H} derived from the cortical thickness features from both HC and AD+ groups. Further, the cortical thickness features in the HC group were z-score normalized and this normalization was used to transform the cortical thickness features of the AD group.

The age prediction ŷᵢ and a vector of residuals rᵢ were obtained for an individual i ∈{1, . . . , 805} in the dataset. The size of residual vector rᵢ was 148×1 and hence, each element of rᵢ corresponded to a distinct brain region as defined by the DKT brain atlas with 148 parcellations. By evaluating the vector of residuals rᵢ for every individual in the combined dataset, a population of residual vectors from HC group (referred to as r_{HC}) and AD+ group (referred to as r_{AD+}) was constructed. The elements of these residual vectors are referred to as regional residuals throughout this document.

Each dimension of these residual vectors was investigated for group differences between HC and AD+ groups via ANOVA as described in Section 3.2. Thus, for every VNN model, we eventually performed m=148 number of ANOVA tests and evaluated the brain regions for significance in group differences in their respective residuals. The significance of group differences between the distributions of regional residuals for HC and AD+ groups corresponding to a brain region was determined after correcting the p-values of ANOVA test for multiple comparisons via Bonferroni correction (Bonferroni corrected p-value <0.05). The group differences were additionally investigated for significance at an uncorrected level using ANCOVA with age and sex as covariates.

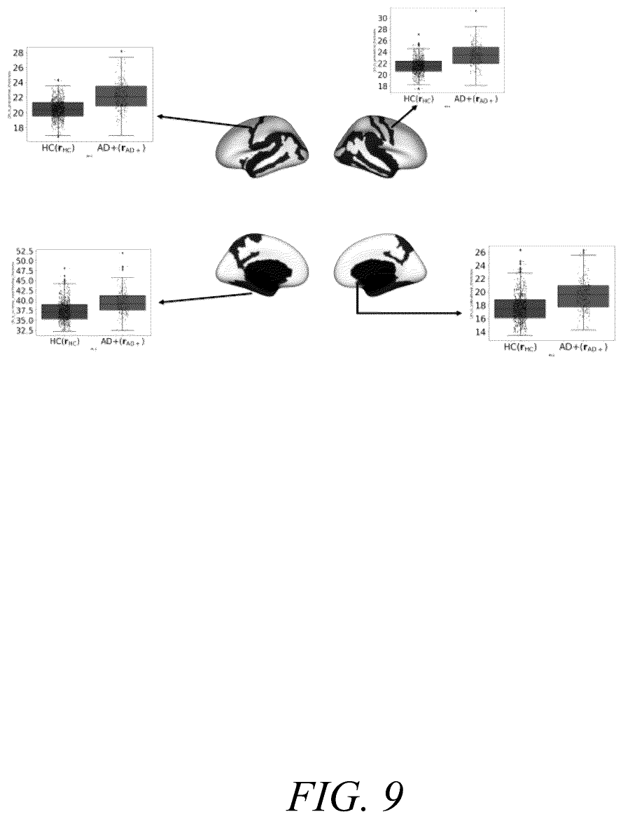

FIG. 9 illustrates the results obtained via ANOVA in this context. The brain regions deemed significant according to the criteria provided in Section 3.2 have been shaded. The box plots for various brain regions show that the regional residuals were significantly elevated in AD+ group as compared to HC. The regional residuals that lead to the results in FIG. 9 are provided in the supplementary material (model ID 50) as part of the regional residuals extracted from all 100 VNN models that were trained on HC group in OASIS-3.

We had 100 trained VNN models for the OASIS-3 dataset and performed similar analyses for each of them. Further, we counted the number of models for which the above described analysis yielded a brain region to be significant. A brain region with robust group difference in its regional residual distribution in HC vs AD+ was expected to be more frequently labeled as significant by the VNN models. The results of this robustness analyses on the OASIS-3 dataset are shown in FIG. 3, Panel a.

### Appendix H: Cross-Validation on ADNI-1 Dataset

In this section, we provide results on the standardized 3.0 T ADN11 dataset (see [34] for details), consisting of 47 controls (age=75.06+3.93 years, 29 females), 71 individuals with mild cognitive impairment (age=74.03±8.12 years, 26 females), and 33 individuals with dementia (age=75.08±8.07 years, 22 females) from the from the wider ADNI database. We chose this standardized dataset in the spirit of reproducibility and to avoid selection bias. The individuals with mild cognitive impairment and dementia diagnosis were combined to form the AD+ cohort, equivalent to that of the AD+ cohort for OASIS-3.

Data used from ADNI database were obtained from the Alzheimer's Disease Neuroimaging Initiative (ADNI) database. The ADNI was launched in 2003 as a public-private partnership, led by Principal Investigator Michael W. Weiner, MD. The primary goal of ADNI has been to test whether serial magnetic resonance imaging (MRI), positron emission tomography (PET), other biological markers, and clinical and neuropsychological assessment can be combined to measure the progression of mild cognitive impairment (MCI) and early Alzheimer's disease (AD). For up-to-date information, see www.adni-info.org.

Data processing. The MRI images for the 3.0T standardized ADNI-1 dataset at the baseline visit were downloaded from https://adni.loni.usc.edu/. Cortical thickness features (curated according to DKT atlas) were derived using the open-access CAT12 pipeline [76] using their default options. We refer the reader to https://neuro-jena.github.io/cat12-help/for detailed processing steps. All outputs were quality checked visually for errors in grey matter segmentation.

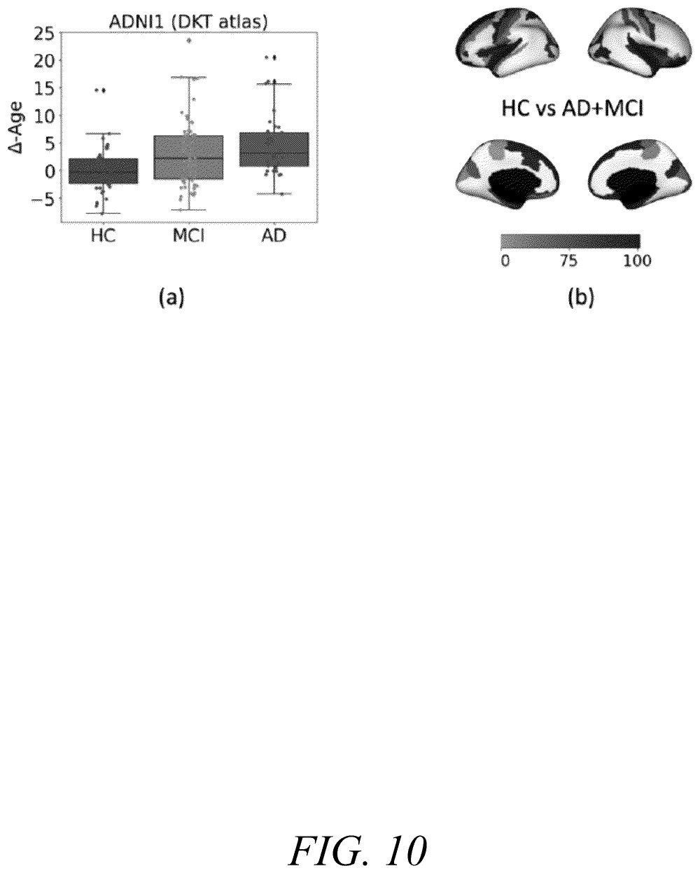

Cross-validation. The results in FIG. 10 were obtained from the models that were trained on OASIS-3 dataset. Here, the anatomical covariance matrix was estimated using cortical thickness measures from all individuals in ADN11 dataset. The observations on ADN11 were highly consistent with those made in the paper. Specifically, in ADN11 dataset, brain age gap was significantly higher for individuals with dementia as compared to healthy controls, with MCI in between them. Also, the subcallosal, entorhinal, temporal pole, and superior temporal regions were cross-validated on this dataset as being contributors to elevated Δ-Age in the combined cohort of dementia and MCI (equivalent to AD+ group from OASIS-3) with respect to the HC group.

### Appendix I: Additional Details on Brain Age Prediction in OASIS-3

In this section, we provide additional figures and discussions pertaining to the results for interpretable brain age prediction in FIG. 3. FIG. 11 displays the distributions of chronological age for AD+ and HC groups in OASIS-3 dataset.

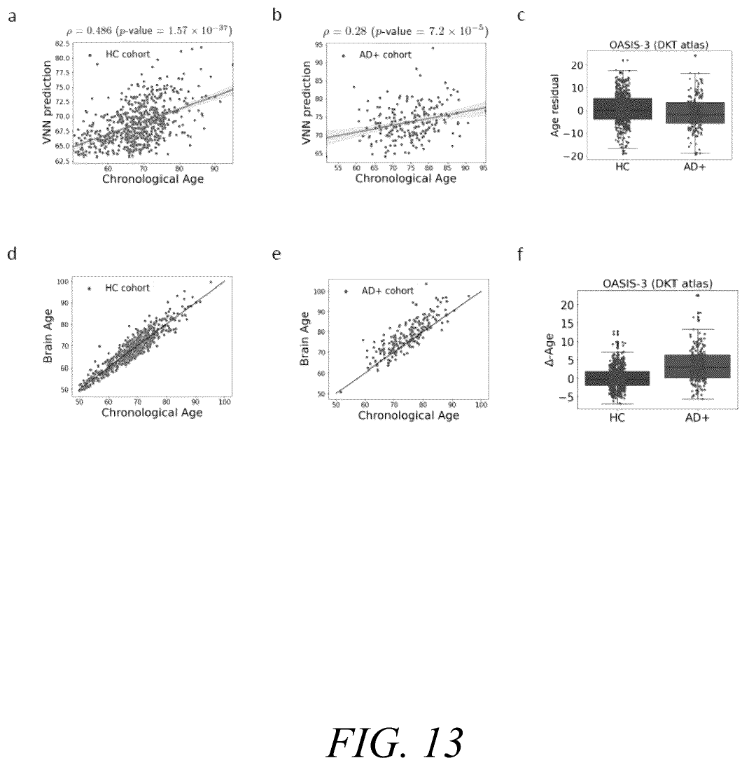

Since VNNs were trained for the regression task, a VNN processed cortical thickness data and provided an estimate for the chronological age for each individual. Since we trained 100 VNN models on different permutations of the training set in OASIS-3, we use the mean of all VNN estimates as the VNN prediction for an individual. This VNN prediction is further leveraged to form brain age estimates and Δ-Age metrics. FIG. 13, Panel a displays the plot for VNN predictions versus chronological age (ground truth) for the complete HC group. The Pearson's correlation between VNN prediction and chronological age (ground truth) for HC group was 0.486, which was similar to that reported in Section 4.1. VNN outputs clearly under-estimated the chronological age for older individuals and over-estimated the chronological age for individuals on the younger end of the age distribution for HC group.

FIG. 13, Panel b displays the plot for VNN predictions versus chronological age (ground truth) for the complete AD+ group. The Pearson's correlation between VNN prediction and chronological age (ground truth) for AD+ group was 0.28. We further note that the VNN architecture and our analysis of regional residuals helped quantify the contribution of each brain region to a data point in FIG. 13, Panel a and FIG. 13, Panel b. Hence, the scatter plot in FIG. 13, Panel b could be affected by larger contributions of certain brain regions for AD+ group relative to the HC group.

FIG. 13, Panel c illustrates the box plots of residuals evaluated by the difference between VNN predictions and chronological age for HC and AD+ groups. FIG. 13, Panel c suggests that the chronological age for AD+ group was underestimated as compared to that for HC group. This observation was also expected since AD+ group is significantly older than the HC group. However, we expect that the robust elevated regional residuals from brain regions in FIG. 3, Panel b mitigated the under-estimation effect due to higher age of AD+ group to some extent.

FIGS. 13, panels d-f display the results after age-bias correction is applied to the VNN outputs. As expected, the brain age for HC group in FIG. 13, panel d is largely concentrated around the line of equality (x=y line). In contrast, the brain age for AD+ group in FIG. 13, panel e is concentrated above the line of equality. These effects manifest into the box plots for Δ-Age in FIG. 13, panel f where we observe the AD+ group to have elevated A-Age as compared to HC group.

VNN architecture facilitated isolation of the effects of accelerated aging before age-bias correction was applied. Hence, the transformation of VNN outputs to brain age from FIG. 13, panels a-c to FIG. 13, panels d-f was not surprising. However, such insights may be infeasible for machine learning approaches that lack transparency and hence, the impact of deviations due to neurodegeneration from the healthy control population cannot be interpreted or isolated. In this context, if the learning model was a black box, FIG. 13 Panels a-c may appear to be counter-intuitive to the goal of detecting accelerated aging in the AD+ group and the effect of age-bias correction can be unclear, thus leading to several criticisms.

### Appendix J: Adaptive Readouts May Penalize the Interpretability of Regional Residuals and Δ-Age

Thus far, we have focused on VNNs that operate with a non-adaptive readout (unweighted average) function. However, it is expected that the performance of the VNNs on their original task of chronological age prediction could be improved significantly with the help of an adaptive readout function. Our experiments showed that this was indeed the case. If a single-layer fully connected perceptron consisting of 10 neurons was added to the VNNs with the same architecture as the ones that were trained on OASIS-3 dataset, we could improve the performance on the chronological prediction task. For 100 VNNs with adaptive readout that were trained on random permutations of the training data, the median MAE for the HC group was 4.64 years, which was significantly smaller than the MAE achieved by VNNs with non-adaptive readouts (Section 4.1). Among the 100 VNN models with adaptive readouts, we analyzed the regional residuals for one VNN model with adaptive readout that had the best performance on chronological age prediction in HC group (test set: MAE=4.17 years, Pearson's correlation between prediction and ground truth=0.73; complete HC group: MAE=4.26 years, Pearson's correlation between prediction and ground truth=0.725). Our regional residuals revealed no significant difference between the regional residuals for AD+ group and HC group. This observation suggested that VNN lost its interpretability due to the addition of adaptive readout function. Moreover, we also observed a diminished gap between Δ-Age for AD+ and HC groups determined using this VNN model (Δ-Age for AD group: 1.58±4.67 years, A-Age for HC group: 0±3.45 years, Cohen's d=0.384). The findings discussed here suggest that boosting the performance on chronological age prediction task by using an adaptive readout function may penalize the interpretability offered by VNNs with non-adaptive readouts and also diminish the Δ-Age gap between AD+ and HC groups.

### Appendix K: Results for Randomly Initialized VNNs

FIG. 14 illustrates results derived by VNN models that were randomly initialized and had the same architecture as those in Section 3.1.

### Appendix L: Regional Profiles Corresponding to Elevated Regional Residuals in AD+ Group are Stable to the Composition of Data Used to Estimate Anatomical Covariance Matrix C_{HA}

Recall that Δ-Age and associated regional profiles were evaluated using VNNs that operated upon a composite anatomical covariance matrix C_{HA}. We next checked whether the results derived from VNNs relevant to A-Age were stable to the changes in composition of the combined HC and AD+ groups used to estimate the anatomical covariance matrix. Note that the bilateral parahippocampal, entorhinal, subcallosal, and temporal pole regions are expected to be among the most relevant to Δ-Age in AD based on the results in FIG. 4.

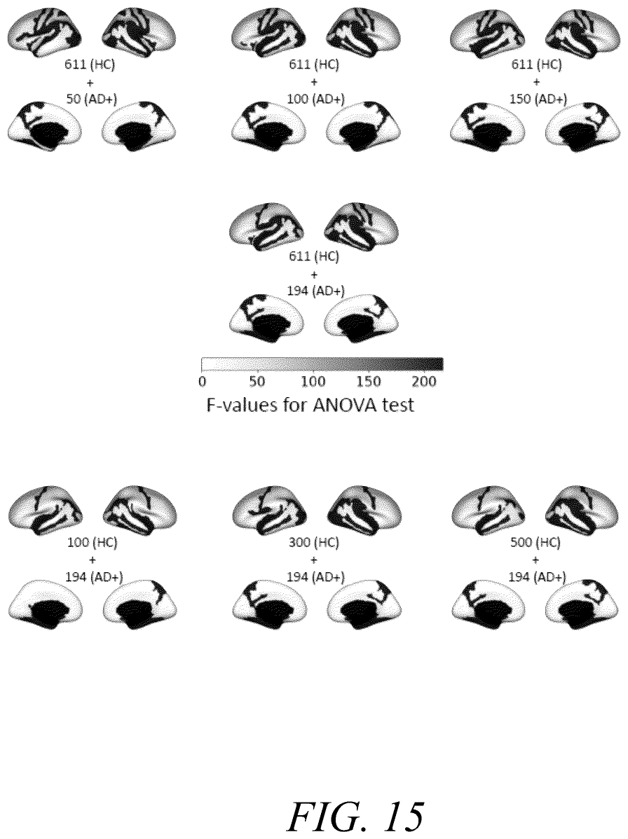

We performed two sets of experiments. In the first set, we included the whole HC group and gradually varied the number of individuals from the AD+ group to be included to form the estimate C_{HA}. FIG. 15, Panel a includes the results obtained from a randomly selected VNN model corresponding to the anatomical covariance matrix formed by different combinations of the individuals from HC and AD+ groups. The results in FIG. 15, Panel a, display the brain regions whose regional residuals from AD+ group were higher than that in the HC group (Bonferroni corrected p-value <0.05). The result obtained by the VNN when it used C_{HA} estimated from all 611 HC individuals and 194 AD+ individuals forms the baseline to evaluate stability in this context. When the covariance matrix C_{HA} was perturbed by using a smaller number of individuals from the AD+ group to estimate it, we observed that the significance of the relevant brain regions (parahippocampal, subcallosal and temporal pole) were preserved till exclusion of about 144 AD+ individuals from the estimate C_{HA}. Hence, the significant differences observed in the regional residuals for AD+ and HC groups in the aforementioned regions were robust to perturbations in C_{HA} due to variability in the number of individuals from the AD+ group.

FIG. 15, Panel b illustrates the results obtained for a similar experiment as above, with the difference that the regional residuals were evaluated for the VNN when the anatomical covariance matrix C_{HA} was perturbed by reducing the number of individuals from the HC group used to estimate it. Using the result obtained for C_{HA} estimated from 611 HC individuals and 194 AD+ individuals in FIG. 15, Panel a as the baseline, the results pertaining to ANOVA between regional residuals for AD+ and HC groups (with AD+ elevated as compared to HC) remained consistent as long as 100 or more individuals from HC group were included in forming C_{HA}. With less than 100 number of HC individuals included in C_{HA}, the results became noticeably less significant in precuneus and supramarginal regions in the left hemisphere.

In summary, the group differences observed between the regional residuals for AD+ and HC groups in OASIS-3 dataset were robust to perturbations in the covariance matrix C_{HA} when it was perturbed from the baseline by using a different combination of HC and AD+ individuals to estimate it. However, we also remark that (nearly) complete exclusion of HC or AD+ groups from C_{HA} resulted in loss of significance of the elevation in regional residuals in AD+ for various regions, including bilateral parahippocampal and temporal pole regions, and precuneus and supramarginal regions in the left hemisphere. Thus, both HC and AD+ groups were relevant to the anatomical covariance matrix C_{HA} that resulted in regional profiles in FIG. 3, Panel a, and they were robust to the combination of individuals from HC and AD+ used to estimate C_{HA}.

### Appendix M: Δ-Age Evaluation with Anatomical Covariance Matrix from HC Group

Using the anatomical covariance matrix derived only from the HC group (denoted by C_{H}) resulted in observations that were consistent with FIG. 3 with a slightly diminished difference in Δ-Age between HC and AD+ groups. Specifically, Δ-Age for AD+ group in this setting was 3.41±4.57 years, which was significantly larger than that for the HC group (ANOVA: partial η²=0.141, Cohen's d=0.88). The correlation between Δ-Age and CDR sum of boxes scores in the AD+ group was 0.464. Thus, when the anatomical covariance matrix derived solely from the HC group was utilized in our brain age prediction framework, the magnitude of Δ-Age and its utility as a marker of dementia severity was slightly diminished as compared to the results in FIG. 3.

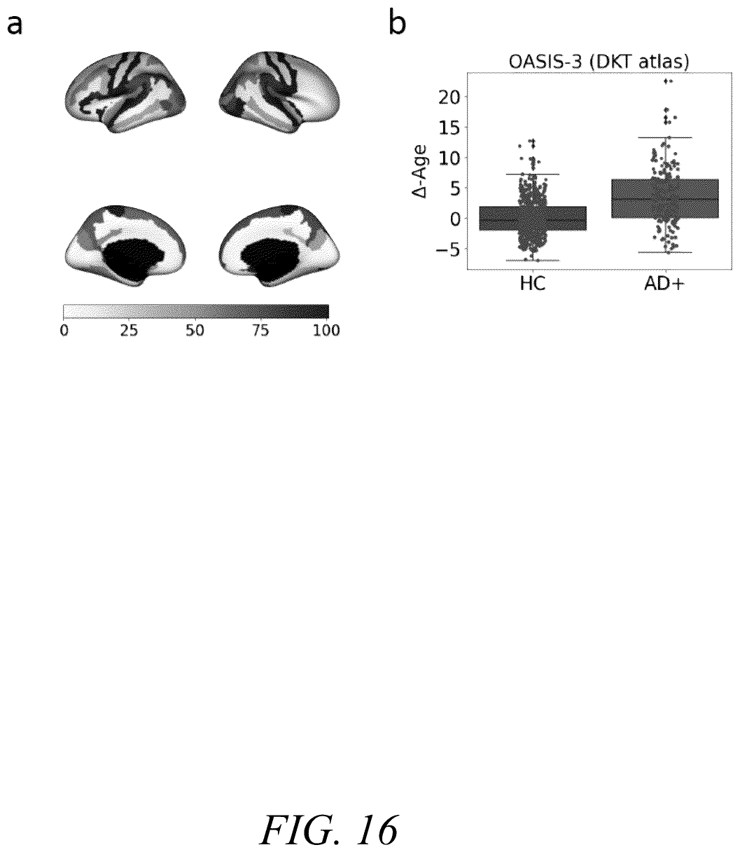

FIG. 16, Panel a displays the regional profile associated with Δ-Age derived from VNNs with C_{H} as the anatomical covariance matrix. Comparison with FIG. 3, panel a reveals that the robustness of regional residuals being elevated in the AD+ group in subcallosal and temporal regions was preserved, but that in bilateral entorhinal and parahippocampal regions was diminished in this scenario. FIG. 16, Panel b displays the distributions for Δ-Age in AD+ and HC groups.

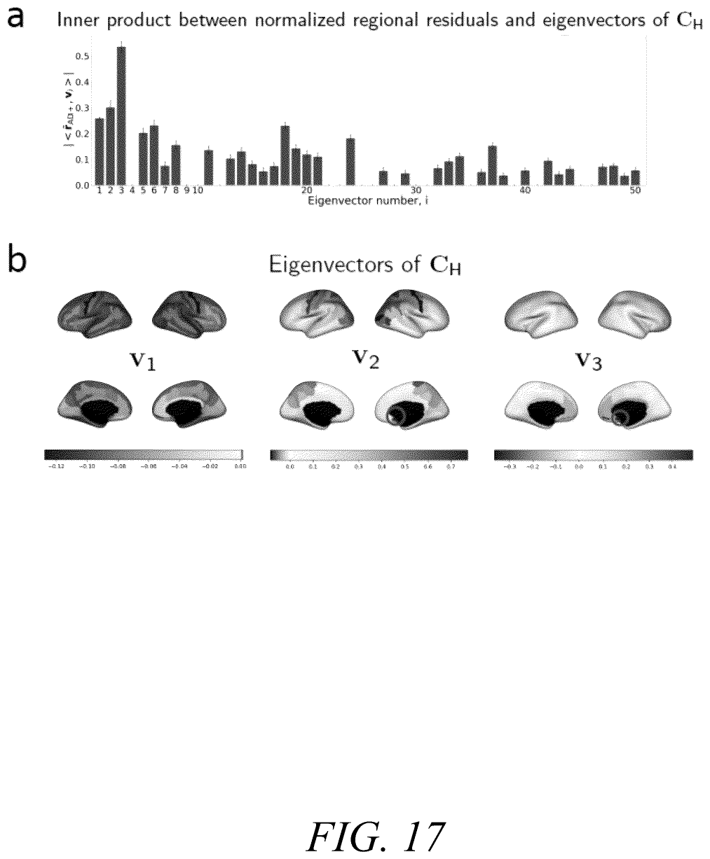

The variation in the regional residuals associated with entorhinal and parahippocampal regions in FIG. 16, Panel a and FIG. 3, panel a could be explained by investigating the eigenvectors of C_{H}. Specifically, FIG. 17, panel a displays the associations between regional residuals for the AD+ group and the first 50 eigenvectors of C_{H}, where we observed that the third, second, and first eigenvectors of C_{H} had the top three largest associations. FIG. 17, panel b plots the projections of the first three eigenvectors of C_{H} on a brain template. Note that the eigenvectors v₃ and v₂ have the largest weights associated with the subcallosal region in the right hemisphere, which is consistent with the relevant eigenvectors of C_{HA} in FIG. 4. However, unlike the eigenvectors of C_{H} in FIG. 17, Panel b, the eigenvectors of C_{HA} in FIG. 4, Panel b are also characterized by comparatively larger weights in the entorhinal and parahippocampal regions. Since parapippocampal and entorhinal regions are well known to be associated with disease onset and cortical atrophy, the anatomical covariance matrix C_{HA} may provide a more holistic perspective to Δ-Age in AD.

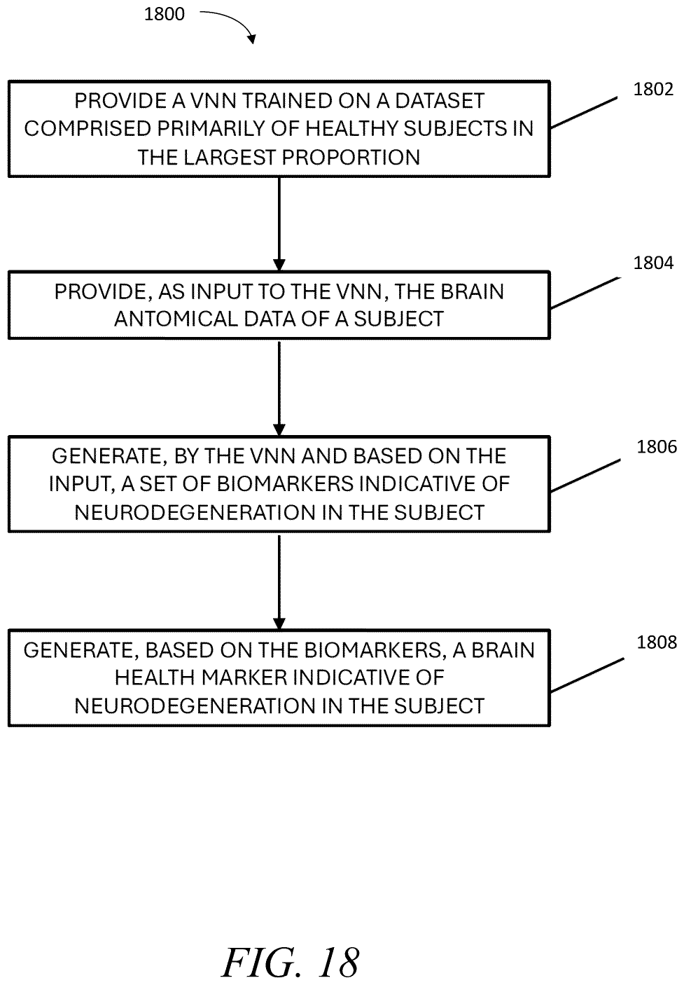

FIG. 18 is a flow diagram of an example method 1800 for identifying biomarkers and a brain health marker indicative of neurodegenerative disease using a VNN. At step 1802, a VNN trained for specific tasks on brain anatomical data from a population comprising of healthy subjects in the largest proportion and an anatomical covariance matrix is provided. Examples of tasks for which VNN is trained for can include predicting the chronological age and predicting the brain activity in the next time step based on the brain activity in the previous time steps. The brain anatomical data used to train the VNN is characterized by multivariate data samples and each element in a data sample is a feature associated with a specific brain region or a combination of brain regions. Examples of brain anatomical data can include brain morphometry features (for example, cortical thickness, volume or surface area) derived from structural magnetic resonance imaging (MRI) images of brains or the time series data derived from functional MRI or EEG modalities of brain imaging from primarily healthy population (healthy subjects form the largest proportion of the dataset). The elements of the anatomical covariance matrix used to train the VNN capture the association between different brain regions. The elements of the anatomical covariance matrix used to train the VNN are determined by the covariance between the features associated with different brain regions or a transformation of the covariance between the features associated with different brain regions. Examples of transformations on the covariance between the features associated with different brain regions can include thresholding, division, normalization, and multiplication.

At step 1804, brain anatomical data of a subject and an anatomical covariance matrix are provided as input to the VNN that has been trained to predict chronological age of healthy population. The brain anatomical data of the subject can include a multivariate data sample and each element in the data sample is a feature associated with a specific brain region or a combination of brain regions. Examples of brain anatomical data can include brain morphometry features (for e.g., cortical thickness, volume or surface area) derived from structural magnetic resonance imaging (MRI) images of brains or the time series data derived from functional MRI or EEG modalities of brain imaging from a dataset is primarily composed of healthy population, making up the largest proportion. The elements of the anatomical covariance matrix capture the association between different brain regions. The elements of the anatomical covariance matrix are determined by the covariance between the features associated with different brain regions or a transformation of the covariance between the features associated with different brain regions. Examples of transformations on the covariance between the features associated with different brain regions can include thresholding, division, normalization, and multiplication.

At step 1806, the VNN generates a set of biomarkers indicative of neurodegeneration of the subject based on the input. Generating the biomarkers indicative of neurodegeneration of the subject can include the VNN outputs or a mathematical transformation of the VNN outputs and mapping the VNN outputs or a mathematical transformation of the VNN outputs to the specific brain regions. Examples of mathematical transformation of the VNN outputs can include weighted or unweighted aggregation, thresholding, and normalization.

At step 1808, the biomarkers of the subject generate a brain health marker indicative of the neurodegeneration of the subject. Example of the brain health marker can include brain age and brain age gap of the subject and a label for the subject, which classifies the subject as healthy or any other set of categories that represent a neurodegenerative condition.

The VNN-based paradigm can generate the brain health marker of the subject from the biomarkers generated by the VNN. The biomarkers generated by the VNN can be associated with the anatomical regions of the subject's brain and their contribution to the brain health marker can be identified. Identification of the anatomical regions contributing to the brain health marker can include evaluating a statistic from the VNN output for each anatomical region that characterizes the anatomical region with respect to the brain health marker.

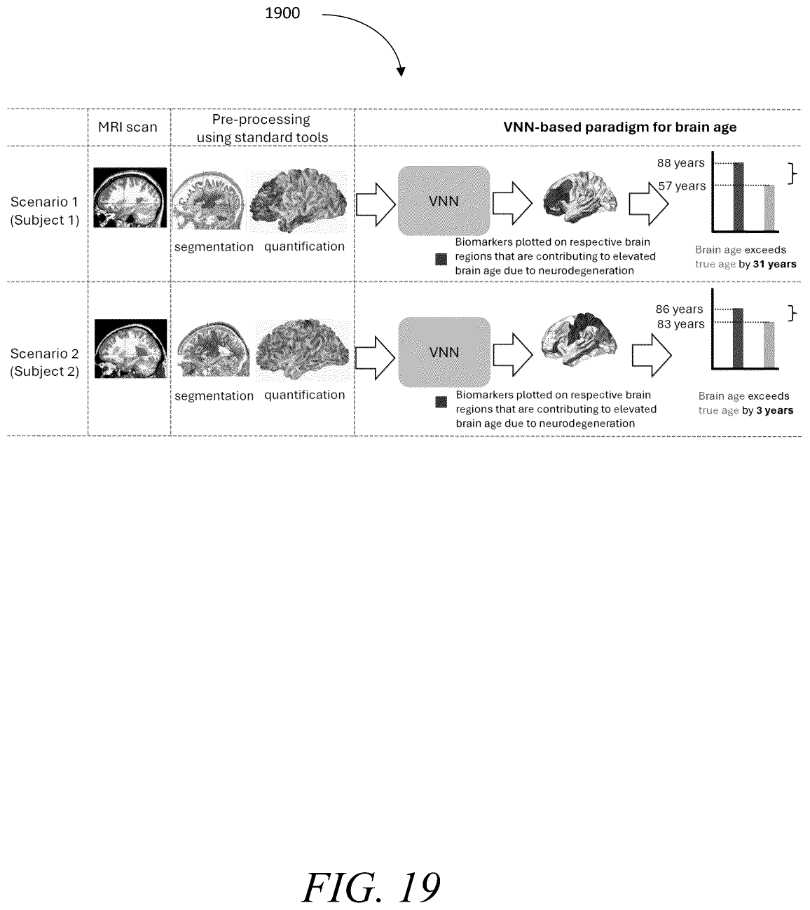

FIG. 19 is a flow diagram illustrating the example process 1900 for deploying a VNN model to identify biomarkers and brain age as a brain health marker of neurodegeneration in two scenarios. In scenario 1, the whole brain cortical thickness data extracted from T1 MRI image for the subject 1 is processed by the VNN to generate outputs of the same dimensionality as the input. The VNN outputs are biomarkers and the biomarkers that are statistically higher than what is expected for a healthy subject are plotted on the brain surface. The VNN outputs are aggregated and transformed through a linear regression model to form the estimate of brain age. In scenario 2, the whole brain cortical thickness data extracted from T1 MRI image for the subject 2 is processed by the same VNN as the scenario 1 to generate outputs of the same dimensionality as the input. The VNN outputs as biomarkers characterize the impact of neurodegeneration in subject 2 through comparisons relative to healthy population are plotted on the brain surface. The VNN outputs are aggregated and transformed through a linear regression model to form the estimate of brain age. Because the VNN can transfer across different subjects with distinct impacts of neurodegeneration to provide brain health marker, this VNN-based paradigm is termed as the foundation model for finding markers of neurodegeneration and providing brain health assessment from brain anatomical data.

### Transferability of Covariance Neural Networks

Graph convolutional networks (GCN) leverage topology-driven graph convolutional operations to combine information across the graph for inference tasks. In our recent work, we have studied GCNs with covariance matrices as graphs in the form of coVariance neural networks (VNNs) that draw similarities with traditional principal component analysis (PCA) while overcoming its limitations regarding instability. In this paper, we focus on characterizing the transferability of VNNs. The notion of transferability is motivated from the intuitive expectation that learning models could generalize to “compatible” datasets (i.e., datasets of different dimensionalities describing the same domain) with minimal effort. VNNs inherit the scale-free data processing architecture from GCNs and here, we show that VNNs exhibit transferability of performance (without re-training) over datasets whose covariance matrices converge to a limit object. Multi-scale neuroimaging datasets enable the study of the brain at multiple scales and hence provide an ideal scenario to validate the transferability of VNNs. We demonstrate the quantitative transferability of VNNs over a regression task based on a multi-scale dataset of cortical thickness features. Further, to elucidate the advantages offered by VNNs in neuroimaging data analysis, we also deploy VNNs as regression models in a pipeline for “brain age” prediction from cortical thickness features. The discordance between brain age and chronological age (“brain age gap”) can reflect increased vulnerability or resilience toward neurological disease or cognitive impairments. The architecture of VNNs allows us to extend beyond the coarse metric of brain age gap and associate anatomical interpretability to elevated brain age gap in Alzheimer's disease (AD). We leverage the transferability of VNNs to cross validate the anatomical interpretability offered by VNNs to brain age gap across datasets of different dimensionalities.

### I. Introduction

In various modern applications, the number of features (denoted by m) in a dataset is a fundamental component of data acquisition that is typically a characteristic of the desired application and logistics involved [1], [2]. Most machine learning algorithms and statistical inference approaches are designed over a pre-defined feature set and hence, their computational and sample complexities inherently depend on the dimensionality m [3], [4]. In this document, we study a deep learning framework called coVariance neural networks (VNN) [5] that is based on graph neural networks (GNNs) operating on the sample covariance matrix from a given dataset and is scale-free, i.e., the number of learnable parameters in VNN is independent of the dimensionality m of the dataset. The scale-free aspect of VNNs makes it feasible for them to be transferable between datasets of different dimensionalities without any changes to their architecture, i.e., VNNs trained on a dataset with dimensionality m=m₁ can process another dataset with dimensionality m=m₂ with the same set of learned parameters. Thus, the notion of transferability of VNNs in this paper is primarily focused on transferability across datasets of different dimensionalities, under the same domain. Other notions of transferability in machine learning, such as domain transferability or transfer between datasets of different distributions, are not considered in this paper.

VNNs are GCNs with sample covariance matrices as a graph and a polynomial graph filter as convolution operation [5]. Covariance matrices and principal component analysis (PCA) form the foundations of non-parametric analyses in various practical applications that are characterized by spatially distributed, multi-variate data acquisition protocols. Some examples of such applications include neuroimaging [6], computer vision [7], weather modeling [8], traffic flow analysis [8], and cloud computing [10]. Moreover, GCNs admit the properties of stability to topological perturbations and transferability across graphs of different sizes in various settings [11]-[14], which makes them a well-motivated data analysis tool for graph-structured data. Our results in [5] formalized the following significant observations: i) there exist similarities between the spectral analysis of graph convolution on a covariance matrix and the standard PCA transformation; and ii) VNNs are robust to the number of samples used to estimate the sample covariance matrix, thus, overcoming a potential source of instability and irreproducibility of PCA based statistical inference [15], [16].

The transferability of GNNs from training graphs to some compatible family of test graphs has been previously studied from different theoretical perspectives [14], [17]-[19]. The notion of transferability of GNNs broadly encapsulates the intuition that GNNs may be able to retain their performance for some inference task when applied over test graphs (irrespective of the size) that describe the same phenomenon as the training graphs. In this context, the study in [17] considers transferability of GNNs over graphs that represent the discretization of underlying topological spaces. Several studies also consider GNN transferability over graphs that belong to a converging sequence that approaches a limiting object in the asymptote of a large number of nodes [19]. In [18], the similarity between the ego graph distributions (derived from graph topology) formed the workhorse for assessing transferability of GNNs. Transferability of GNNs also provides advantages in terms of computational complexity, which for GNNs scales as O(m²) for dense graphs with m nodes. In this document, we broaden the notion of transferability to VNNs and establish the transference over covariance matrices of different sizes that converge in some sense. In this context, transferability is not feasible for traditional statistical models as they are restricted within the feature space of the original dataset and need to be reevaluated if the number of features change. Thus, transferability of VNNs is broadly relevant to the domain of multivariate statistics.

Neuroimaging is an example of a timely application in which the number of features can vary across datasets, yet different datasets contain similar information [20], [21]. Specifically, MRI data can be represented in many scales ranging from single voxels (typically ˜1 mm³) to regions-of-interest (ROIs) derived from multi-scale brain atlases that range from dozens to thousands of parcellations (e.g., from 100 to 1000 number of parcellations in a multi-scale brain atlas [22], [23]). There has been a growing interest in multi-scale datasets in neuroscience [21], [24]-[27]. These datasets rely on brain atlases or templates that allow a multi-scale parcellation of the brain surface (for instance, Schaefer's atlas and Lausanne atlas). A multi-scale brain atlas partitions the brain cortex into a variable number of regions at different scales. However, statistically sound approaches that can leverage or cross-validate the redundancy of information in datasets at multiple scales are currently lacking.

Our recent work described herein has demonstrated that VNNs can provide an anatomically interpretable perspective to the task of “brain age” prediction from cortical thickness features in AD [28]. Accelerated aging (i.e., when brain age estimated from neuroimaging is elevated as compared to chronological age) can be a predictor of cognitive decline or neurological conditions like Alzheimer's disease and related dementias (ADRD) [29], [30]. Hence, in this application, the difference between the brain age estimated from neuroimaging data and the chronological age, i.e., the brain age gap (A-Age) is the metric of interest. Here, we leverage the transferability of VNNs to cross-validate Δ-Age predictions and their associated anatomical interpretability across multi-scale datasets.

Several machine learning and statistical approaches have been adopted to estimate brain age from neuroimaging data [29]-[36]. Commonly, these methods rely on models trained to predict chronological age of healthy population. Deep learning approaches are often adopted due to their ability to provide highly accurate estimates of chronological age in healthy population. However, the accuracy of chronological age prediction in healthy population may not be correlated with the clinical utility of brain age estimates in adverse health conditions [37]. Moreover, due to lack of transparency in such models, it is not guaranteed that relevant disease-driven effects in the neuroimaging data were indeed leveraged in the estimates of brain age. To address this, limited studies have utilized state-of-the-art post-hoc, model-agnostic methods such as SHAP or LIME [36] and saliency maps [35] to add explainability to their brain age estimation approaches, identifying the input features most relevant to the inference outcome. However, explainability offered by such approaches may be unstable to small perturbations to the input [38], [38], inconsistent to variations in training algorithms and model multiplicity (i.e., when multiple models with similar performance may exist) [41], and computationally expensive [42]. In this context, VNNs provide a transparent learning model that is inherently interpretable and can associate elevated Δ-Age with brain regions characteristic of a disease or health condition as well as the principal components of the covariance matrix, with no significant added computational cost. Importantly, the implication of the transferability of VNNs is that the anatomical interpretability offered by VNNs can also be guaranteed on datasets of different dimensionalities; a feature which is infeasible for state-of-the-art explainability methods.

Contributions. Our contributions can be summarized as follows:
Transferability of VNNs: We theoretically characterize the transferability of VNNs between datasets of different dimensionalities. For a dataset with m₁ features and covariance matrix Cₘ_{₁} and another dataset with m₂ features and covariance matrix Cₘ_{₂}, we establish that the outputs of a VNN when instantiated on Cₘ_{₁} and Cₘ_{₂} are close in some sense under appropriate conditions on the covariance matrices Cₘ_{₁} and Cₘ_{₂} (see Theorem 2).
Evaluation on multi-scale neuroimaging datasets: We train VNNs for the regression task of predicting chronological age from cortical thickness features. The transferability of VNNs is validated by the transference of regression performance across multi-scale cortical thickness datasets curated according to different scales of a commonly used brain atlas (Section IV-B). Further, in Section IV-C we deploy VNNs in the pipeline for anatomically interpretable brain age prediction from and compare the Δ-Age between healthy controls and individuals with AD diagnosis. We leverage the transferability of VNNs to cross-validate the distributions of Δ-Age and the accompanying anatomical interpretability across cortical thickness datasets available at different scales of a multi-resolution brain atlas. In the interest of reproducibility, the code developed is publicly available at https://github.com/sihagsNNN_Brain_Age.

As described above, we have empirically demonstrated transferability of VNNs on a regression task in [5] and a brain age prediction task described above. However, no theoretical results regarding transferability were provided in these preliminary studies. Moreover, our prior work in did not associate brain age with anatomical interpretability. This document extends the contributions in [5] and those described above in various significant ways. First, we develop a theoretical framework to establish transferability of VNNs. Also, a more comprehensive empirical evaluation in the context of brain age is provided, where we leverage the transferability of VNNs to cross-validate anatomical interpretability across datasets of different dimensionalities.

### II. coVariance Neural Networks

VNNs operate on covariance matrices and have similar architecture as GNNs (GCNs and GNNs are used interchangeably herein). We start by providing preliminary definitions pertaining to the architecture and discuss the theoretical properties associated with VNNs later.

Consider an m-dimensional random vector x ∈ [symbol] ^{m×1} whose ensemble covariance matrix is defined as

$$C {=}^{Δ} 𝔼 [ x - 𝔼 [ x ] {( x - 𝔼 [ x ] )}^{⊤} ] \qquad ( 1 )$$

Where ^{T} is the transpose operator and [symbol] [·] is the expectation with respect to the probability distribution of x. In practice, we usually have access to a dataset that provides us with the statistical information about x. Therefore, we also consider a dataset consisting of n random, independent and identically distributed (i.i.d) samples of x, given by xᵢ∈ [symbol] ^{m×1}, ∀i ∈{1, . . . , n}, where the dataset can be represented in matrix form as Xₙ=[x₁, . . . , xₙ]. Using Xₙ, the sample covariance matrix estimator is given by

$$\hat{C} {=}^{Δ} \frac{1}{n - 1} {\sum }_{i = 1}^{n} ( {x}_{i} - \overline{x} ) {( {x}_{i} - \overline{x} )}^{⊤} \qquad ( 2 )$$

where x̅ is the sample mean. Next, we discuss the motivation behind studying VNNs separately from GNNs.

### A. Motivation

Covariance matrices are ubiquitous in real world applications that have spatially distributed, multi-variate data acquisition protocols [6], [8]-[10]. The eigenvectors of covariance matrices are termed as principal components of the dataset and constitute the PCA transformation [43].

The covariance matrix C can be viewed as the adjacency matrix of a graph representing the stochastic structure of the vector x, where the m dimensions of x can be thought of as the nodes of an m-node, undirected graph and its edges represent the pairwise covariance between elements in x. Furthermore, the eigenvalues of C encode the variability of the dataset along different directions in an orthogonal space determined by the associated eigenvectors or principal components.

In graph signal processing, a vector defined on the nodes of the graph is viewed as the graph signal and the projection of a graph signal on the eigenbasis of the graph yields the graph Fourier transform [44]. The graph Fourier transform provides a systematic mathematical tool to analyze convolutional filters over graphs [45], [46]. Interestingly, the classical Fourier transform and graph Fourier transform converge over a discrete, periodic time series represented on a directed, cyclic graph [47]. Similarly to the graph Fourier transform, we can define the coVariance Fourier transform as the projection of a random instance x on the eigenvectors of the covariance matrix C [5]. For ease of exposition, we henceforth use the notation x to refer to a realization of the random vector whose covariance matrix is C. The definition of coVariance Fourier transform from [5] is stated next. For this purpose, consider the eigendecomposition of C given by

$$C = V Λ {V}^{⊤} , \qquad ( 3 )$$

where V=[v₁, . . . , vₘ] is a matrix of size m×m with its columns as the eigenvectors and Λ=diag(λ₁, . . . , λₘ) is a diagonal matrix with its diagonal elements representing the eigenvalues of C.
Definition 1 (coVariance Fourier Transform). The coVariance Fourier transform (VFT) of a random sample x is defined as its projection on the eigenspace of C and is given by

$$\tilde{x} {=}^{Δ} {V}^{⊤} x . \qquad ( 4 )$$

The i-th entry of \tilde{x}, i.e., [\tilde{x}]ᵢ represents the projection of x on eigenvector vᵢ and hence, it is associated with the eigenvalue λᵢ. Thus, the similarity between PCA transformation and VFT in (4) implies that eigenvalue λᵢ encodes the variability of the dataset Xₙ in the direction of the principal component vᵢ. In this context, the eigenvalues of the covariance matrix are the mathematical equivalent of the notion of graph frequencies in graph signal processing.

GNNs with convolutional filters that rely on a linear shift-and-sum operation fundamentally exhibit the stability to changes in graph topology[13]. Since the sample covariance matrix Ĉ is likely to be perturbed with respect to C [48], stability is desirable to mitigate finite-sample effects on statistical inference. Motivated by this observation, we define the notion of coVariance filters that are polynomials in the covariance matrix and characterize the convolution operation in VNNs.
Definition 2 (coVariance Filters). Given a set of real valued, scalar parameters

$$ℋ = {\{ {h}_{k} \}}_{k = 0}^{K} ,$$

the coVariance filter on a covariance matrix C is defined as

$$ℋ ( C ) {=}^{Δ} {{\sum }_{k = 0}}^{K} {h}_{k} {C}^{k} . \qquad ( 5 )$$

Furthermore, the output of the covariance filter H(C) for an input x is given

$$by z = H ( C ) x . \qquad ( 6 )$$

The application of coVariance filter H(C) on an input x translates to combining information across different sized neighborhoods.

The spectral analysis of the covariance filtering operation in Definition 2 via VFT of the filter output z yields the frequency response of the covariance filter and reveals the similarities between covariance filtering and PCA. After taking the VFT of z, we have

$$\tilde{z} = {V}^{⊤} H ( C ) x = {\sum }_{k = 0}^{K} {h}_{k} {Λ}^{k} {V}^{⊤} x = {\sum }_{k = 0}^{K} {h}_{k} {Λ}^{k} \tilde{x} \qquad ( 7 )$$

where \tilde{x}=V^{T}x is the covariance Fourier transform of x and (7) is valid due to the orthonormality of eigenvectors of C. The frequency response of the coVariance filter depends on the filter taps [symbol] ={hₖ} as well as the eigenvalues of C, and is given by

$$h ( λ ) = {\sum }_{k = 0}^{K} {h}_{k} {λ}^{k} . \qquad ( 8 )$$

Furthermore, since x is a projection of x on the eigenvector space V and [\tilde{z}]ᵢ=h(λᵢ)[V^{T}x]ᵢ, the i-th element of \tilde{z} exhibits similarities with the standard PCA transformation. This observation is formalized in Lemma 1.

Lemma 1 (Spectrum of coVariance Filter and PCA). Given a covariance matrix C with eigendecomposition in (3), if the PCA transformation of input x is given by q=V^{T}x, there exists a filter bank of coVariance filters {Hᵢ(C): i∈ {1, . . . , m}}, such that, the score of the projection of input x on eigenvector vᵢ can be recovered by the application of a coVariance filter Hᵢ(C) as:

$${[ q ]}_{i} = {v}_{i}^{⊤} {H}_{i} ( C ) x \qquad ( 9 )$$

where the frequency response hᵢ(λ) of the filter Hᵢ(C) is given by

$${h}_{i} ( λ ) = \{ \begin{matrix}1 , & if λ = {λ}_{i} , \\ 0 , & otherwise\end{matrix} . \qquad ( 10 )$$

Lemma 1 asserts the equivalence between processing data samples with a weighted PCA transformation and with a specific polynomial on the covariance matrix.

Our work in [5] showed that in contrast to PCA involving eigenvectors of the sample covariance matrix, information processing with polynomials of the sample covariance matrix can be stable to finite sample-induced perturbations. Indeed, in practice we may only have access to the sample covariance matrix Ĉ which is an estimate of C. Since Ĉ is a consistent estimator of C, the eigenvalues and eigenvectors of Ĉ approach those of C in the limit of infinite number of samples, i.e., n→∞. However, for finite n, the eigenvectors and eigenvalues of Ĉ are perturbed with respect to those of C [48]. In principle, statistical inference using PCA can be prone to instability due to eigenvectors corresponding to eigenvalues of the ensemble covariance matrix that are close [15] and, thus, lead to irreproducible statistical conclusions [16]. In this context, we informally state Theorem 2 from [5].

Lemma 2 (Stability of coVariance filter). Consider a dataset with sample covariance matrix Ĉ formed by n samples and the counterpart ensemble covariance matrix C. Under a mild assumption in [5], the following holds with high probability:

$$‖ H ( \hat{C} ) - H ( C ) ‖ = 𝒪 ( \frac{v}{{n}^{\frac{1}{2} - ε}} ) , \qquad ( 11 )$$

for some ν>0 and ε∈ (0, 1/2).

Lemma 2 establishes that information processing using a polynomial of the covariance matrix offers stability with respect to the perturbations between the sample covariance matrix C and C [5]. Also, as a corollary to Lemma 2, we can state that the difference between outputs of covariance filters instantiated on distinct sample covariance matrices are bounded. These observations imply that statistical inference based on covariance filters is robust to finite sample size effects and, thus, result in consistent statistical outcomes with high confidence. No such guarantees are offered by approaches that leverage PCA-based transformation. Next, we discuss VNN architectures based on covariance filters, which results in VNNs inheriting the stability offered by coVariance filters.

### B. Architecture

We begin with the description of a coVariance perceptron that forms one layer of the VNN architecture. To this end, we leverage a pointwise, nonlinear activation function σ(·), such that, for x=[x₁, . . . , xₘ], we have σ(x)=[σ(x₁), . . . , σ(xₘ)]. Examples of pointwise, nonlinear activation functions are ReLU and tanh.
Definition 3 (coVariance Perceptron). For a given pointwise, nonlinear activation function σ(·), input x, a coVariance filter H(C) and its corresponding set of filter taps [symbol] the coVariance perceptron is defined as

$$Φ ( x ; C , ℋ ) {=}^{△} σ ( H ( C ) x ) . \qquad ( 12 )$$

A VNN can be constructed by stacking perceptrons to form a multi-layer information processing architecture. This observation is formalized next. Remark 1 (Multi-layer VNN). Consider an L-layer architecture formed by stacking L coVariance perceptrons defined in Definition 3. In this scenario, we denote the coVariance filter in layer [symbol] by H [symbol] (C) and its corresponding set of filter taps are given by [symbol] For a given pointwise nonlinear activation function σ(·), the relationship between the input [symbol] and the output [symbol] for the coVariance perceptron in the, [symbol] -th layer is given by

$${x}_{ℓ} = σ ( {H}_{ℓ} ( C ) {x}_{ℓ - 1} ) for ℓ ∈ \{ 1 , … , L \} , \qquad ( 13 )$$

where x₀ is the input x. We refer to this L-layer architecture as an L-layer VNN.

A figurative illustration of a multi-layer VNN is included in FIG. 7. Furthermore, sufficient expressive power can be accommodated in the VNN architecture via multiple input multiple output (MIMO) processing at every layer. Formally, consider a VNN layer [symbol] that can process [symbol] number of m-dimensional inputs and outputs [symbol] number of m-dimensional outputs via [symbol] × [symbol] number of filter banks. In this scenario, the input is specified as Xᵢₙ=[xᵢₙ[1], . . . , Xᵢₙ[Fᵢₙ]], and the output is specified as Xₒᵤₜ=[xₒᵤₜ[1], . . . , xₒᵤₜ[Fₒᵤₜ]]. The relationship between the f-th output xₒᵤₜ[f] and the input xᵢₙ is given by

$${x}_{out} [ f ] = σ ( {\sum }_{g = 1}^{{F}_{i n}} {H}_{fg} ( C ) {x}_{i n} [ g ] ) , \qquad ( 14 )$$

where H_{fg}(C) is the coVariance filter that processes xᵢₙ[g]. Without loss of generality, we focus the subsequent discussion on the scenario when we have [symbol] =F, ∀ [symbol] ∈{1, . . . , L}. In this case, the set of all filter taps is given by

$$ℋ = \{ {ℋ}_{fg}^{ℓ} \} , ∀ f ,$$

g ∈{1, . . . , F}, [symbol] ∈{1, . . . , L}, where

$${ℋ}_{fg} = {\{ {h}_{fg}^{ℓ} [ k ] \}}_{k = 0}^{K} and {h}_{fg}^{ℓ} [ k ]$$

is the k-th filter tap for filter H_{fg}(C). Thus, we can compactly represent a multi-layer VNN architecture capable of MIMO processing via the notation Φ(x; C, [symbol] ), as the set of filter taps [symbol] capture the full span of its architecture. We also use the notation Φ(x; C, [symbol] ) to denote the output of the VNN. For a VNN with F number of m-dimensional outputs in the final layer, the size of the VNN output Φ(x; C, [symbol] ) is m×F. The output Φ(x; C, [symbol] ) is succeeded by a readout function that maps Φ(x; C, [symbol] ) to the desired output. In this paper, we adopt a non-adaptive or non-learnable readout function (e.g., mean, max or min functions), which preserves the property of permutation invariance for the VNN model. Furthermore, a non-adaptive readout function is essential for the transferability property of VNNs (discussed in Section III).

It is prudent to study the robustness of VNN outputs to the number of samples n in order to guarantee reproducibility of VNN statistical outcomes. Specifically, it is desirable that the change in VNN outputs is controlled or bounded when the architecture is trained using sample covariance matrices estimated from n₁ or n₂ samples, when n₁≠n₂. In Theorem 1, we informally state the result on the stability of VNNs by analyzing ∥Φ(x; Ĉ, [symbol] )-Φ(x; C, [symbol] )∥, i.e., the operator norm of the difference between the VNN outputs for the sample covariance matrix Ĉ and the ensemble covariance matrix C. This Theorem was also previously established in [5].

Theorem 1 (VNN Stability). Consider an ensemble covariance matrix C and its estimate Ĉ formed from n samples. Given a bank of coVariance filters with filter taps

$$ℋ = \{ {ℋ}_{fg}^{ℓ} : f ,$$

g ∈{1, . . . , F}, [symbol] ∈{1, . . . , L}}, the coVariance filters are stable under a mild assumption in and satisfy

$$‖ {ℋ}_{fg}^{ℓ} ( \hat{C} ) - {H}_{fg}^{ℓ} ( C ) ‖ \leq {α}_{n} , \qquad ( 15 )$$

for some αₙ>0 with high probability (Lemma 2). Also, for a pointwise, nonlinear activation function σ(·), such that, |σ(a)-σ(b)|≤| a-b| for a, b∈ [symbol] , we have

$$‖ Φ ( x ; \hat{C} , ℋ ) - Φ ( x ; C , ℋ ) ‖ \leq {LF}^{L} {α}_{n} . \qquad ( 16 )$$

The parameter αₙ in (15) represents the finite sample effect on the perturbations in Ĉ with respect to C, and is borrowed from Lemma 2. By leveraging the perturbation theory of covariance matrices to analyze the stability of coVariance filters, we also show in the proof of Theorem 1 that αₙ scales as

$$1 / {n}^{\frac{1}{2} - ε} ,$$

for some ε∈ (0, 1/2). We note that the bound in (16) becomes looser as F or L increases, which is consistent with the (less refined) results for GNNs. However, without the analysis of lower bounds on ∥Φ(x; Ĉ, [symbol] )-Φ(x; C, [symbol] )∥, we cannot claim that the robustness of VNNs indeed worsens with an increase in F or L. Moreover, we remark that VNNs sacrifice discriminability between eigenvectors associated with close eigenvalues to achieve stability [5]. As a corollary, we also state that Theorem 1 can readily be extended to characterize the difference between VNN outputs corresponding to sample covariance matrices estimated from n₁ and n₂ samples, via (16) and application of the triangle inequality.

### III. Transferability of VNNs

The notion of transferability of VNNs across datasets of different dimensionalities is made feasible by the “scale-free” property of coVariance filters (Definition 2). From an implementation perspective, transferability of VNNs to a dataset of different number of features amounts to replacing the covariance matrix C in a VNN model Φ(·; C, [symbol] ) with a covariance matrix of another size, while keeping the parameters [symbol] fixed. Since we consider covariance matrices of different dimensionalities, we denote a covariance matrix C of size m×m by Cₘ. Informally, we can state our objective for assessing transferability as follows.

Informal problem statement for VNN transferability. Given a data point xₘ_{₁} from a dataset with m₁ features and associated covariance matrix Cₘ_{₁}, and another data point xₘ_{₂} from a dataset with m₂ features and associated covariance matrix Cₘ_{₂}, we aim to characterize the operator distance between VNN outputs Φ(xₘ_{₁}; Cₘ_{₁}, [symbol] ) and Φ(xₘ_{₂}; Cₘ_{₂}, [symbol] ). If Φ(xₘ_{₁}; Cₘ_{₁}, [symbol] ) and Φ(xₘ_{₂}; Cₘ_{₂}, [symbol] ) converge in some sense, we can conclude that the parameters [symbol] are transferable between two datasets consisting of m₁ and m₂ features.

a. Continuous Representation of a VNN

Note that the VNN outputs Φ(·; Cₘ_{₁}, [symbol] ) and Φ(·; Cₘ_{₂}, [symbol] ) have distinct dimensionalities if m₁≠m₂ and therefore, a direct comparison between them is not natural. Fundamentally, it is imperative to provide a mathematical framework to compare vectors and covariance matrices of different sizes in order to facilitate transferability analyses of VNNs. To this end, we consider a simple mapping that represents the vector on a continuous interval [0, 1].

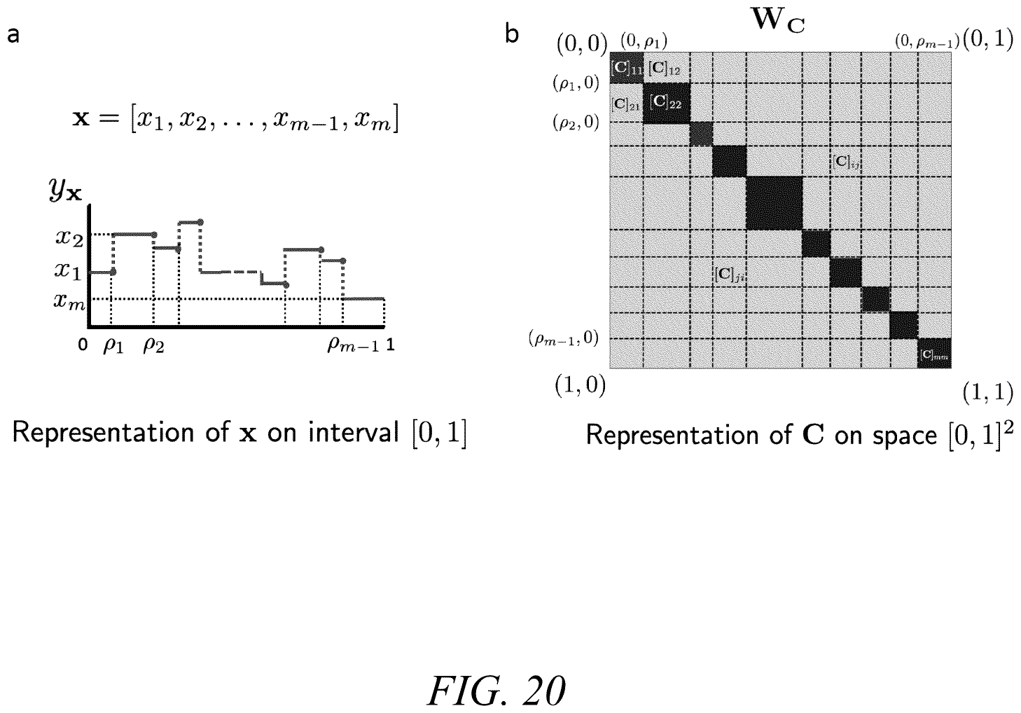

Specifically, given an m-dimensional vector x=[x₁, . . . , xₘ], we can define a continuous representation of x as a function yₓ: [0,1] [symbol] [symbol] such that yₓ(u)=xᵢ for u ∈ [symbol] ᵢ, where [symbol] ᵢ is a pre-defined interval associated with the i-th element of x. Similarly, we can map a covariance matrix Cₘ to a compact set [0, 1]² using the mapping W_{C}_{ₘ}: [0,1]² [symbol] [symbol] where we have W_{C}_{ₘ}(u, V)=[Cₘ]ᵢⱼ for u∈ [symbol] ᵢ and v∈ [symbol] ⱼ. A pictorial illustration of yₓ and W_{C} for covariance matrix C is included in FIG. 20. Therein, the intervals [symbol] ᵢ are parameterized by variables ρᵢ, which will be discussed subsequently in (18). Note that we can recover x from yₓ and vice-versa (similarly for Cₘ and W_{C}_{ₘ}). Hence, for data points xₘ_{₁} and xₘ_{₂} consisting of m₁ and m₂ elements, respectively, the closeness of continuous representations

$${y}_{{x}_{{m}_{1}}} and {y}_{{x}_{{m}_{2}}}$$

can be used as a metric to assess the similarity between data points in multi-scale datasets. This observation also extends to the comparison between matrices Cₘ_{₁} and Cₘ_{₂}.

For a VNN architecture with F outputs in the final layer, the dimensionality of VNN outputs Φ(xₘ_{₁}; Cₘ_{₁}, [symbol] ) is m₁×F and for Φ(xₘ_{₂}; Cₘ_{₂}, [symbol] ) it is m₂×F. Thus, we can compare Φ(xₘ_{₁}; Cₘ_{₁}, [symbol] ) and Φ(xₘ_{₂}; Cₘ_{₂}, [symbol] ) in terms of the continuous representations of every column in outputs Φ(xₘ_{₁}; Cₘ_{₁}, [symbol] ) and Φ(xₘ_{₂}; Cₘ_{₂}, [symbol] ), where these continuous representations are defined in the same fashion as yₓ above. For VNN Φ(xₘ_{₁}; Cₘ_{₁}, [symbol] ), we use the notation yₘ_{₁}[f] to denote the continuous representation of the f-th output in Φ(xₘ_{₁}; Cₘ_{₁}, [symbol] ), i.e.,

$${y}_{{[ Φ ( {x}_{{m}_{1}} ; {C}_{{m}_{1}} , ℋ ) ]}_{f}} .$$

Similar to the relationship between yₓ and x, the f-th VNN output [Φ(xₘ_{₁}; Cₘ_{₁}, [symbol] )]_{f} and its continuous representation yₘ_{₁}[f] are operationally interchangeable (see the Appendix for details). Using the continuous representations above, we can now formally describe the assessment of transferability of VNNs.

Formal problem statement for VNN transferability: Consider two VNNs Φ(xₘ_{₁}; Cₘ_{₁}, [symbol] ) and Φ(xₘ_{₂}; Cₘ_{₂}, [symbol] ) instantiated on data with m₁ and m₂ features, respectively. If we have the following conditions: (a) the continuous approximations of inputs xₘ_{₁} and xₘ_{₂} are close, i.e.,

$${‖ {y}_{{x}_{{m}_{1}}} - {y}_{{x}_{{m}_{2}}} ‖}_{2}$$

is bounded; and (b) the continuous approximations of covariance matrices Cₘ_{₁} and Cₘ_{₂} are close, i.e.,

$${‖ {W}_{{C}_{{m}_{1}}} - {W}_{{C}_{{m}_{2}}} ‖}_{2}$$

is bounded; we aim to characterize the closeness between the continuous representations of VNN outputs Φ(xₘ_{₁}; Cₘ_{₂}, [symbol] ) and Φ(xₘ_{₂}; Cₘ_{₂}, [symbol] ), i.e., find ϑ>0, such that,

$${‖ {y}_{{m}_{1}} [ f ] - {y}_{{m}_{2}} [ f ] ‖}_{2} \leq ϑ , ∀ f ∈ \{ 1 , … , F \} . \qquad ( 17 )$$

### B. Mathematical Foundations of Transferability

The continuous representations of graph signals and graphs have previously been leveraged to study transferability of GNNs under the domain of graphon information processing [49]. Specifically, GNNs can be transferable between graphs belonging to a converging sequence if the graphs in this sequence converge to a limit object called graphon, as the number of nodes approaches infinity [50]. In a similar fashion, we leverage the theory of graphons [50] and graphon signal processing [49] to establish the transferability of VNNs. Graphons are the limits of dense graphs (i.e., graphs with number of edges of the order θ(m²)) [52] and hence, appropriate to study limits of covariance matrices that are typically dense. The definition of a graphon is provided in Definition 4
Definition 4 (Graphon). A graphon is a bounded, symmetric, measurable function W: [0, 1]² [symbol] [−1, 1].

To align the covariance matrix with the notion of graphon in Definition 4, we can consider an appropriate scaling procedure (for instance, scaling the covariance matrix such that its maximum eigenvalue is 1). Recalling that a covariance matrix can be viewed as a weighted graph, a sequence of covariance matrices {Cₘ} being convergent implies that the sequence of their continuous representations, i.e., {W_{C}_{ₘ}}, converges to some graphon W if W_{C}_{ₘ} is appropriately constructed from Cₘ. This claim is based on generalizing to our setting and the formal statement is provided in Remark 2. This statement leverages the notion of cut distance to characterize convergence. The definition of the cut distance between any two covariance matrices Cₘ_{₁} and Cₘ_{₂} is borrowed from the definition of cut distance between weighted graphs in [50].

Remark 2 (Graphon as limit object [50]). A sequence of covariance matrices {Cₘ} is deemed convergent if they form a Cauchy sequence with respect to the cut distance [50]. Furthermore, for any convergent sequence of covariance matrices {Cₘ}, the corresponding sequence of graphon approximations {W_{C}_{ₘ}} converges to a graphon W.

### Image

To establish transferability of VNNs, we consider a converging sequence of covariance matrices {Cₘ} and investigate whether the parameters H can be transferred between any two VNNs instantiated on distinct covariance matrices in this sequence. The construction of W_{C}_{ₘ} relies on appropriately defining the intervals [symbol] ᵢ and is described in the following steps [50], Section 3.1].
a. Partition the interval [0,1] into m disjoint intervals [ [symbol] ₁, . . . , [symbol] ₘ], such that,

$${𝒰}_{i} = \{ \begin{matrix}[ 0 , {ρ}_{1} ] if i = 1 \\ [ {ρ}_{i - 1} , {ρ}_{i} ] if i ∈ \{ 2 , … , m \}\end{matrix} , \qquad ( 18 )$$

$$where$$

$${ρ}_{i} \hat{=} \frac{1}{tr ( {C}_{m} )} {\sum }_{j = 1}^{i} {[ {C}_{m} ]}_{jj} \qquad ( 19 )$$

and tr(Cₘ) is the trace of Cₘ. Clearly, ρₘ=1.
b. The relationship between feature i and feature j is given by

$${W}_{{C}_{m}} ( {u}_{i} , {u}_{j} ) = {[ {C}_{m} ]}_{ij} for {u}_{i} ∈ {𝒰}_{i} , {u}_{j} ∈ {𝒰}_{j} .$$

If the continuous representation W_{C}_{ₘ} of Cₘ is constructed according to the above steps, we refer to W_{C}_{ₘ} as the graphon approximation of Cₘ. Thus, the graphon W forms the schema for which the covariance matrix Cₘ represents the covariance realization at resolution m. Our main result on the transferability of VNNs is contingent upon the following assumptions related to the covariance matrix Cₘ, the graphon limit W, and frequency response of the coVariance filters.
A.1 ((Ω, ζ)-dominant property of covariance matrices.) For the sequence {Cₘ}, there exist positive constants Ω and ζ, such that we have

$$\frac{1}{tr ( {C}_{m} )} {\begin{matrix}max \\ j ∈ \{ 1 , … , m \}\end{matrix} [ {C}_{m} ]}_{jj} \leq \frac{Ω}{{m}^{ζ}} , \qquad ( 20 )$$

for all finite m. We refer to the covariance matrix Cₘ satisfying the property in (20) as being (Ω, ζ)-dominant. This property implies that

$$\frac{1}{tr ( {C}_{m} )} {{max}_{j ∈ \{ 1 , … , m \}} [ {C}_{m} ]}_{jj} \to 0 , as m \to ∞ .$$

A.2. (Lipschitz continuity of Graphon.) If W is the limit of the sequence {W_{C}_{ₘ}} as m→∞, then W satisfies

$$\vert W ( {u}_{1} , {v}_{1} ) - W ( {u}_{2} , {v}_{2} ) \vert \leq {α}_{1} ( \vert {u}_{1} - {u}_{2} \vert + \vert {v}_{1} - {v}_{2} \vert ) , \qquad ( 21 )$$

for any u₁, v₁, u₂, v₂ ∈[0,1] and α₁>0. Any graphon satisfying (21) is termed an α₁-Lipschitz graphon. The Lipschitz continuity of graphon W determines the smoothness of the information present between any two coordinates (u₁, v₁) and (u₂, v₂).
A.3. The graphon approximation W_{C}_{ₘ} and the limit W satisfy W_{C}_{ₘ}(ρᵢ, ρⱼ) W(ρᵢ,ρⱼ), where ρᵢ is defined in (19).
A.4. (Lipschitz inputs to VNNs.) The continuous approximations of the inputs to VNNs are α₂-Lipschitz for some α₂>0. Given an input xₘ_{₁}, its continuous approximation y_{X}_{ₘ}₁ satisfies

$$\vert {y}_{{x}_{{m}_{1}}} ( u ) - {y}_{{x}_{{m}_{1}}} ( v ) \vert \leq {α}_{2} \vert u - v \vert , for u , v ∈ [ 0 , 1 ] . \qquad ( 22 )$$

A.5. (Lipschitz coVariance filters.)
   The frequency response of a coVariance filter is α₃-Lipschitz for some α₃>0, i.e., it satisfies |h(η)-h(\hat{η})|≤α₃|η-\hat{η}| for any pair of scalars (η, \hat{η}).

Assumption A1 suggests that the variance profile of individual features in the dataset, characterized by their corresponding diagonal elements in the covariance matrix, must not be concentrated in a small subset of features. Also, since the covariance matrix Cₘ is considered to be a finite realization of graphon W, a smaller Lipschitz constant α₁ in Assumption A2 implies a smaller information mismatch between W and W_{C}_{ₘ} for a finite m, when W_{C}_{ₘ} and W satisfy the construction in Assumption A3. Assumption A4 characterizes the smoothness of entries across the features of the dataset and, hence, the Lipschitz constant α₂ is a property of the given data. Assumption A5 characterizes the variability in the coVariance filter outputs and is derived from the analyses of the transferability of VNNs.

Next, we state the main result of this section that establishes the transferability between VNNs processing datasets consisting of m₁ and m₂ features.

Theorem 2 (Transferability of VNNs). Consider two VNNs Φ(xₘ_{₁}; Cₘ_{₁}, [symbol] ) and Φ(xₘ_{₂}; Cₘ, [symbol] ) consisting of L layers and F outputs per layer. Under Assumptions A1-A5, the continuous representations of covariance matrices and inputs to the VNNs are close, i.e.,

$${‖ {W}_{{C}_{{m}_{1}}} - {W}_{{C}_{{m}_{2}}} ‖}_{2} \leq {α}_{1} ϱ ( Ω , ζ , {m}_{1} , {m}_{2} ) , \qquad ( 23 )$$

$$and {‖ {y}_{{x}_{{m}_{1}}} - {y}_{{x}_{{m}_{2}}} ‖}_{2} \leq {α}_{2} ϱ ( Ω , ζ , {m}_{1} , {m}_{2} ) . \qquad ( 24 )$$

Moreover, we have

$$‖ {{y}_{{m}_{1}} [ f ] - {y}_{{m}_{2}} [ f ] ‖}_{2} \leq {LF}^{L} αϱ ( Ω , ζ , {m}_{1} , {m}_{2} ) , \qquad ( 25 )$$

for f∈{1, . . . , F}, where

$$ϱ ( Ω , ζ , {m}_{1} , {m}_{2} ) \hat{=} {Ω}^{3 / 2} ( \frac{1}{{m}_{1}^{3 ζ / 2 - 1}} + \frac{1}{{m}_{2}^{3 ζ / 2 - 1}} ) , \qquad ( 26 )$$

α=(α₁(α₃+β)+α₂) for some constant β>0, and ζ∈(2/3, 1]. Proof. See Appendix.

Theorem 2 implies that continuous representations of all F outputs of the respective final layers of VNNs Φ(xₘ_{₁}; Cₘ_{₁}, [symbol] ) and Φ(xₘ_{₂}; Cₘ_{₂}, [symbol] ) converge as m₁ and m₂ grow. Since the continuous representation yₘ_{₁}[f] and VNN output [Φ(xₘ_{₁}; Cₘ_{₁}, [symbol] )]_{f} are operationally interchangeable, we expect the measures of central tendency (e.g., mean, median) of outputs [Φ(xₘ_{₁}; Cₘ_{₁}, [symbol] )]_{f} and [Φ(xₘ_{₂}; Cₘ_{₂}, [symbol] )]_{f} to converge as well. By extension, we also expect the measures of central tendency for Φ(xₘ_{₁}; Cₘ_{₁}, [symbol] ) and Φ(xₘ_{₂}; Cₘ_{₂}, [symbol] ) to converge if Theorem 2 holds. In this context, if the VNN readout function is the unweighted mean, we expect the statistical outcomes of VNNs Φ(xₘ_{₁}; Cₘ_{₁}, [symbol] ) and Φ(xₘ_{₂}; Cₘ_{₂}, [symbol] ) to be close and this convergence to be stronger for large m₁ and m₂. We also remark that Assumption A5 must hold only for a pair of scalars η and \hat{η} that characterize the eigenvalues of the limiting graphon W and the graphon approximation W_{C}_{ₘ} in the proof of Theorem 2 and hence, is less stringent in practice.

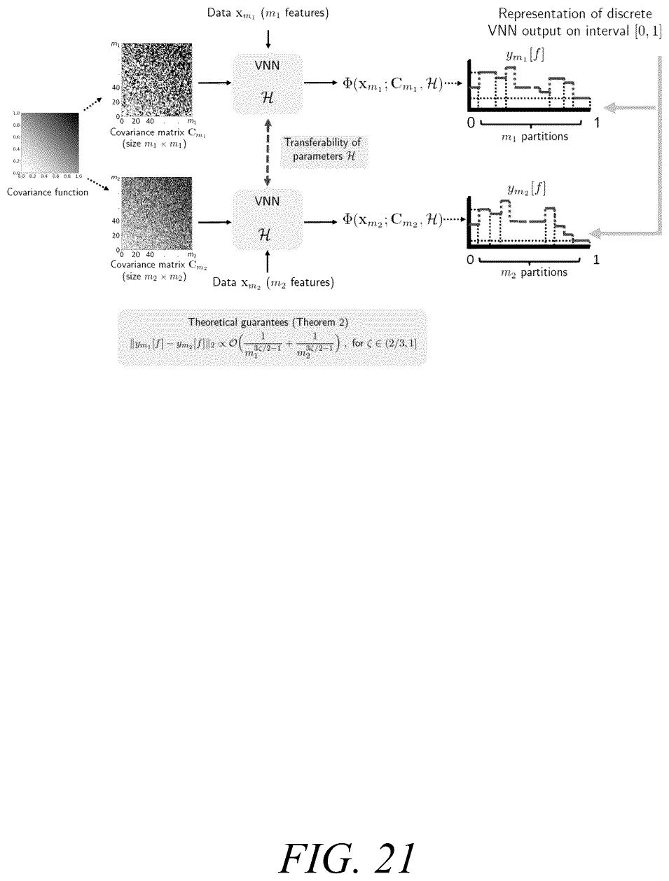

The impact of Theorem 2 is broad, as we have shown that the parameters [symbol] can be “scale-free” while preserving the performance over an inference task. Specifically, a VNN can be instantiated on a dataset of different dimensionality than the training dataset and the VNN recovers close statistical outcomes for the same parameters [symbol] for both datasets, provided the data samples and covariance matrices of the training dataset and the new dataset are close in terms of their continuous representations. Thus, VNNs also offer a significant advantage over PCA-based analysis approaches as the principal components are restricted within the feature space of a dataset. They do not provide any mathematical insight into the structure of another dataset of different dimensionality, even when the datasets may be related. FIG. 21 provides an overview of the transferability of VNNs. Note that the theoretical results in Theorem 2 and the associated Assumptions A1-A5 have been provided in the context of ensemble covariance matrices. In practice, the VNNs operate on sample covariance matrices, which are estimates of the ensemble covariance matrices. Under the inherent statistical uncertainty due to the finiteness of the data, Assumption A3 may not be satisfied exactly for sample covariance matrices. Thus, the sample size and hence the closeness of the sample covariance matrices to the ensemble covariance matrices may also dictate the quality of transferability of VNNs.

As discussed previously, a multi-scale neuroimaging dataset provides an ideal setting for validating the transferability guarantees in Theorem 2. Indeed, note that VNNs also provide feature-level expressivity at the final layer. For instance, if a VNN is deployed for a regression task and the readout layer is a simple mean function, the VNN outputs can be used to characterize the contributions of each feature in the dataset to the regression outcomes. This can be of great value in neuroimaging applications, as each feature in a neuroimaging dataset is typically associated with a distinct brain region. Thus, VNNs naturally provide a feasible way to interpret the final statistical outcomes. Importantly, interpretability offered by VNNs can be traced to individual principal components of the covariance matrix. Hence, we contend VNNs are inherently interpretable, unlike other black-box deep learning architectures that rely on model-agnostic substrates for explainability, such as SHAP [52] or LIME [53]. Moreover, explanations offered by these methods may be unstable [38], [39], inconsistent [40], and computationally prohibitive [42]. Unlike a simpler statistical model such as PCA-based regression, VNNs offer theoretical stability guarantees that are of practical relevance; see [5] for an empirical demonstration of this advantage. We can further intertwine the interpretability with the transferability of VNNs to cross-validate findings across datasets of different dimensionalities. This desirable feature is lacking for most explainability methods in machine learning, which are primarily driven by sensitivity analyses of model outputs to perturbations in the input features. The observations made here motivate well investigating the utility of VNNs on multi-scale neuroimaging datasets; the subject dealt with next.

### IV. Experiments

### A. Multi-Scale FTDC Datasets

These datasets consist of the cortical thickness data extracted at different resolutions from a group of healthy controls (HC; n=105, age=62.6±7.62 years, 57 females) and a group of 67 individuals with mild cognitive impairment or Alzheimer's disease diagnosis (AD+; age=68.52±9.29 years, 28 females). For each individual, the cortical thickness data was curated according to multi-resolution Schaefer atlas [22], at 100 parcel, 300 parcel, and 500 parcel resolutions with finer resolution cortical thickness estimates with increasing number of parcellations. The ANTs cortical thickness pipeline [54], [55] was used to derive mean cortical thickness within each atlas parcel using 3T T1-weighted MRIs (˜1 mm isotropic resolution). We investigate the transferability of VNNs on three datasets: FTDC100, FTDC300 and FTDC500, that constitute the cortical thickness datasets corresponding to 100, 300 and 500 cortical thickness features, respectively. Also, the FTDC100, FTDC300, and FTDC500 datasets are jointly referred to as FTDC datasets. All participants in the FTDC datasets took part in an informed consent procedure approved by an Institutional Review Board convened at University of Pennsylvania. The MRI data for FTDC datasets were provided by the Penn Frontotemporal Degeneration Center (NIH AG066597). Cortical thickness data were made available by Penn Image Computing and Science Lab at University of Pennsylvania. We remark that results consistent with the ones reported here have been obtained on publicly available datasets in recent work [56].

### B. Transferability of VNNs for a Regression Task

VNNs were trained as regression models to predict chronological age using cortical thickness data of the HC group from FTDC datasets across different resolutions of Schaefer's atlas. To begin with, we remark that the sequence of covariance matrices formed by cortical thickness features extracted according to 100,300,500 parcellations for the HC group in the FTDC datasets was converging. This assessment was pertinent as our theoretical results in Theorem 2 hold for a converging sequence of covariance matrices. Our primary objective here is to show that predictive performance is transferable by VNN models across FTDC datasets, without re-training. Hence, this experiment is beyond the scope of traditional multivariate regression approaches that rely on a PCA-based transformation in the first step, followed by a regression model.

VNN learning. We trained three sets of VNN models; one each for the HC group in FTDC100, FTDC300, and FTDC500 datasets. The training process was similar for all VNNs. Note that the VNN output of the architecture represented by Φ(x; Ĉ, [symbol] ) for one m-dimensional input is of dimension m×F if the VNN architecture has F m-dimensional outputs in the final layer. The regression output is determined by a readout layer which evaluates an unweighted mean of all outputs of the final layer of VNN. We use the notation Ĉ for covariance matrix here, as the VNN architecture is instantiated on the sample covariance matrix in practical implementations. Therefore, the regression output for a vector of cortical thickness features x is given by

$$\hat{y} = \frac{1}{F m} {Σ}_{j = 1}^{m} {{Σ}_{f = 1}^{F} [ Φ ( x ; \hat{C} , ℋ ) ]}_{jf} . \qquad ( 27 )$$

Prediction using unweighted mean at the output implies that the VNN model exhibits permutation-invariance (i.e., the final output is independent of the permutation of the input features and covariance matrix) and transferability.

For a regression task, the training dataset

$${\{ {x}_{i} , {y}_{i} \}}_{i = 1}^{n}$$

is used to learn the filter taps in [symbol] for the VNN Φ(·; Ĉ, [symbol] ) such that they minimize the training loss, i.e.,

$${ℋ}_{o p t} = \begin{matrix}min \\ ℋ\end{matrix} \frac{1}{n} {\sum }_{i = 1}^{n} ℓ ( {\hat{y}}_{i} , {y}_{i} ) \qquad ( 28 )$$

where ŷ is evaluated similar to (27) and [symbol] (·) is the mean-squared error (MSE) loss function.

Each dataset was split into an approximately 90/10 train/test split. Thus, the test sets for FTDC datasets consisted of 10 individuals. The sample covariance matrix was evaluated using all samples in the training set (n=95) and we had the sample covariance matrix C of size m×m (where m=100 for FTDC100, m=300 for FTDC300, m=500 for FTDC500). Furthermore, for all datasets, C was normalized such that its maximum eigenvalue was 1. Next, the training set was randomly split internally, such that, the VNN was trained with respect to the MSE loss between the predicted age and the true age in n=84 samples for FTDC datasets. The loss was optimized using batch stochastic gradient descent with Adam optimizer available in the PyTorch library [58] for up to 100 epochs. The batch size was 34 for FTDC100 dataset, 8 for FTDC300 dataset, and 12 for FTDC500 dataset. The VNN model with the minimum MSE on the remaining samples in the training set (which acted as a validation set) was included in the set of nominal models for this permutation of the training set. For each dataset, we trained and validated the VNN models over 100 permutations of the complete training set of n=95 samples for each of the FTDC datasets, thus, leading to 100 trained VNN models (also referred to as nominal models) per dataset.

The hyperparameters for the VNN architecture and learning rate of the optimizer were chosen according to a hyperparameter search procedure using the package Optuna [59]. For FTDC100, the VNN had a L=2-layer architecture, with a filter bank such that we had F=26 and 2 filter taps in each layer. The learning rate for the optimizer was 0.058. The number of learnable parameters for this VNN was 1456. For FTDC300, the VNN had a L=2-layer architecture, with a filter bank such that we had F=39 and 3 filter taps in the first layer and 2 filter taps in the second layer. The learning rate for the optimizer was 0.0241. The number of learnable parameters for this VNN was 3237. For FTDC500, the VNN model had a L=2-layers with a filter bank such that we had F=27 and 4 filter taps in the first layer and 2 filter taps in the second layer. The number of learnable parameters for this VNN was 1620. The learning rate for the Adam optimizer was set to 0.0631.

Since the readout layer in all trained VNNs was non-adaptive and it evaluated the unweighted mean of the outputs of the final VNN layer to form an estimate for chronological age, the trained VNN could readily process a dataset with different dimensionality without any retraining or alteration to the architecture. The performance outcomes were quantified in terms of mean absolute error (MAE) and Pearson's correlation between the VNN output and ground truth.

Results. We tabulate MAE in Table I and Pearson's correlation between ground truth and VNN output in Table II. Since the objective is to illustrate transferability of VNNs over different scales, the MAE and Pearson's correlation results are reported for complete datasets. For both tables, the row ID provides the dataset on which VNN models were trained and the column ID indicates the dataset for which the VNN performance is reported (after transferring the VNNs if training and testing datasets are different). For instance, the element with row ID “FTDC100” and column ID “FTDC300” in Table I represents the mean and standard deviation of MAE evaluated on FTDC300 dataset (m=300) for the 100 nominal VNN models trained on FTDC100 dataset (m=100). The elements with same row ID and column ID in Tables I and II provide the baseline performance to gauge performance after transferring VNNs.

TABLE I
Transferability across datasets (MAE for VNN regression
outputs with respect to the ground truth)

| | Testing | | |
|---|---|---|---|
| Training | FTDC100 (HC) | FTDC300 (HC) | FTDC500 (HC) |
| FTDC100 (HC) | 5.39 ± 0.084 | 5.5 ± 0.101 | 5.61 ± 0.132 |
| FTDC300 (HC) | 5.39 ± 0.193 | 5.41 ± 0.167 | 5.47 ± 0.169 |
| FTDC500 (HC) | 5.43 ± 0.2 | 5.38 ± 0.15 | 5.4 ± 0.169 |

TABLE II
Transferability across datasets (Pearson's correlation
between VNN outputs and ground truth).

| | Testing | | |
|---|---|---|---|
| Training | FTDC100 (HC) | FTDC300 (HC) | FTDC500 (HC) |
| FTDC100 (HC) | 0.49 ± 0.017 | 0.47 ± 0.018 | 0.468 ± 0.018 |
| FTDC300 (HC) | 0.498 ± 0.05 | 0.49 ± 0.042 | 0.486 ± 0.04 |
| FTDC500 (HC) | 0.51 ± 0.021 | 0.509 ± 0.02 | 0.51 ± 0.021 |

The results in Tables I and II show that the performance of VNNs in terms of MAE and correlation between VNN output and ground truth was preserved after transferring VNNs across FTDC datasets that were curated according to different resolutions of Schaefer's atlas. We also remark that this experiment is not feasible for PCA-regression models as the principal components and the regression model from one dataset cannot be naively transferred to process another dataset that has a different number of features. Next, we demonstrate the utility of regression models trained in Section IV-B in the task of anatomically interpretable brain age prediction. Besides sacrificing the transferability property of VNNs, the usage of adaptive readout functions can also potentially impact the interpretability offered by VNNs in the context of brain age prediction (Appendix I in [28]). Hence, VNNs trained as regression models without adaptive readout functions can provide an explainable perspective to an inference task, albeit without achieving the best possible performance.

### C. Transferability of Interpretability in Δ-Age Prediction Task

Δ-Age is a known biomarker of cognitive decline and neurodegeneration [29], [33]. Also, age is a major risk factor for Alzheimer's disease (AD) and hence, AD is characterized by biological traits that signify accelerated aging [60]. We start by leveraging our Δ-Age prediction pipeline from [28] to provide an anatomical perspective to Δ-Age in AD in FTDC100 dataset.
1) Δ-Age prediction in FTDC100: A layman's overview of the procedure of inferring Δ-Age using neuroimaging data can be summarized in three steps.
Step 1. Train a VNN model to predict chronological age of a healthy population from a neuroimaging dataset.
Step 2. If the correlation between chronological age estimate and ground truth is smaller than 1, it may induce an age-related bias in the VNN model output (implying underestimation for older individuals and overestimation for younger individuals). Hence, an age-bias correction model (e.g., using linear regression) is applied to correct for this bias in the VNN model outputs.
Step 3. The output of the VNN model after age-bias correction forms the brain age estimate. The difference between the brain age estimate and chronological age provides the Δ-Age for an individual.

In Step 1, we utilize the VNN models trained to predict chronological age for FTDC100 group in Section IV-B. Because the regression output by the VNN model was determined by unweighted aggregation of the final layer outputs, it can be conceptualized as an unweighted mean of age predictions at individual brain regions. Therefore, the VNN architecture allows us to compute “regional residuals” (scalar output at a given region derived from VNN final layer output—aggregated VNN output or age estimate formed by VNN) at each brain region to assess their contribution to the final output of VNN. This procedure is formalized next.

Identification of regions associated with neurodegeneration. The VNN architecture allows us to associate a scalar output with each of the m dimensions in the final layer. Specifically, we have

$${p}_{i} = \frac{1}{F} {{Σ}_{f = 1}^{F} [ Φ ( {x}_{i} ; {\hat{C}}_{m} , ℋ ) ]}_{f} , \qquad ( 29 )$$

where pᵢ is the vector denoting the mean of all final layer outputs associated with filters in the filter bank at the final layer. Note that the mean of all elements of pᵢ is the prediction ŷᵢ formed in (27). In the context of cortical thickness datasets, we can associate each element of pᵢ with a distinct brain region. Therefore, the vector pᵢ is a vector of “regional contributions” to the output ŷᵢ by the VNN. The parameters H were learnt over the HC group as described previously and kept unchanged in the subsequent analyses. We use the notation Ĉ₁₀₀ for the covariance matrix formed by the cortical thickness features from HC group.

Next, we leverage (29) to capture the effect of neurodegeneration on brain regions. For this purpose, in the FTDC100 dataset, we evaluated the covariance matrix

$${\hat{C}}_{100}^{A D +}$$

from the combined cortical thickness data of HC and AD+ groups. Note that the VNN models were not re-trained for Δ-Age evaluations and hence, were oblivious to the AD+ group during training. For every individual in the combined dataset of HC and AD+ groups, we processed their cortical thickness data x through the VNN model

$$Φ ( x ; {\hat{C}}_{100}^{A D +} , ℋ ) ,$$

where parameters [symbol] were learnt in the regression task on the data from HC group as described previously. Hence, the mean vector of all final layer outputs for cortical thickness input x is given by

$$p = \frac{1}{F} {{Σ}_{f = 1}^{F} [ Φ ( x ; {\hat{C}}_{100}^{A D +} , ℋ ) ]}_{f} , \qquad ( 30 )$$

and the VNN output is

$$\hat{y} = \frac{1}{100} {{Σ}_{j = 1}^{100} [ p ]}_{j} .$$

Furthermore, we define the vector of residuals as r, whose α-th element (associated with brain region represented by feature α in this case) is given by

$${[ r ]}_{a} {=}^{△} {[ p ]}_{a} - \hat{y} . \qquad ( 31 )$$

Thus, (31) allows us to characterize the residuals with respect to the VNN output ŷ at the regional level for an individual with cortical thickness data x. Henceforth, we refer to the residuals evaluated according to (31) as “regional residuals”. We hypothesized that a larger brain age in a neurodegenerative condition could be linked to an aggregated effect of contributions from certain biologically plausible brain regions.

The population of residual vectors for HC group is denoted by [symbol] _{HC} and that for individuals in AD+ group by [symbol] _{AD+}. ANOVA was performed to test for [symbol] group differences between the regional residuals from the individuals in HC and AD+ groups. Also, since the objective is to capture accelerated aging, our results focus only on elevated regional residual distribution in AD+ group with respect to HC group. Further, the group difference between AD+ and HC groups in the residual vector element for a brain region was deemed significant if it met the following criteria: i) the corrected p-value (Bonferroni correction) for the clinical diagnosis label in the ANOVA model was smaller than 0.05; and ii) the uncorrected p-value for clinical diagnosis label in ANCOVA model with age and sex as covariates was smaller than 0.05.

An example for this regional analysis of VNN outputs is included in [28, Appendix F]. The analysis of regional residuals described above was performed for each trained VNN model, and we tabulated the number of VNN models for which a brain region was deemed to have a higher regional residual in the AD+ group with respect to the HC group. A higher number of VNN models isolating a brain region as significant suggested higher robustness of the effect observed for that brain region.

The fsbrain package in R was used to project the robustness of significantly elevated regional residuals for a brain region on the brain template [61]. Subject-level brain age prediction.[brain_age] In general, the systemic bias in the gap between ŷ and y, where the age may be underestimated for older individuals and overestimated for younger individuals, may confound the interpretations of brain age [61]. Therefore, to correct for this age-driven bias, we adopted a linear regression model based approach to correct any age bias in the VNN age estimates. Specifically, the VNN estimate ŷ was bias-corrected to obtain the brain age ŷ_{B} for an individual with chronological age y and cortical thickness data x, by adopting the following procedure.
Step 1. Fit a linear regression model on the complete dataset to determine coefficients α and β in the following linear model: ŷ−y=αy+β.
Step 2. Evaluate brain age as follows: ŷ_{B}=ŷ−(αy+β). For an individual with cortical thickness x and chronological age y, the brain age gap Δ-Age is defined as

$$Δ - Age {=}^{△} {\hat{y}}_{B} - y . \qquad ( 32 )$$

The linear regression model in the age-bias correction procedure was trained only for the HC group to account for bias in the VNN estimates due to healthy aging, and then applied to the AD+ group. Further, the distributions of Δ-Age were obtained for all individuals in HC and AD+ groups. We verified that differences in Δ-Age for AD+ and HC group were not driven by age or gender differences via ANCOVA with age and sex as covariates.

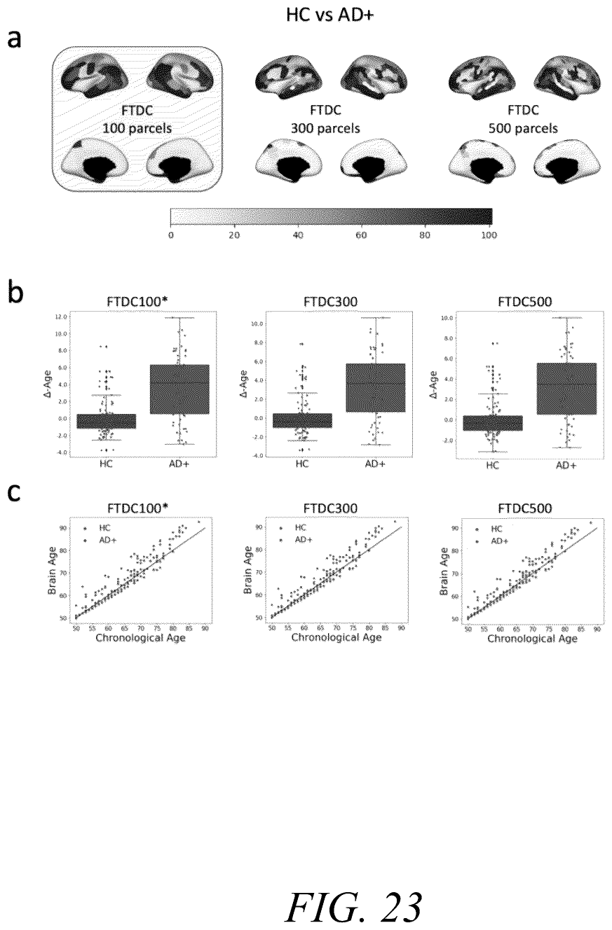

Results. The brain regions with significantly elevated regional residuals in AD+ with respect to HC are projected on the brain template for the FTDC100 dataset in FIG. 23, Panel a. Specifically, FIG. 23, Panel a displays the robustness (from analyses of 100 VNN models) for various brain regions in having an elevated regional effect in their corresponding residual elements for AD+ group with respect to HC group. The highlighted brain regions were concentrated in various biologically plausible regions for AD, such as bilateral medial temporal, temporal pole, entorhinal, and frontal regions. Additional experiments showed that the anatomical interpretability inferred by the analyses of residual elements was highly correlated with specific eigenvectors of

$${\hat{C}}_{100}^{A D +} .$$

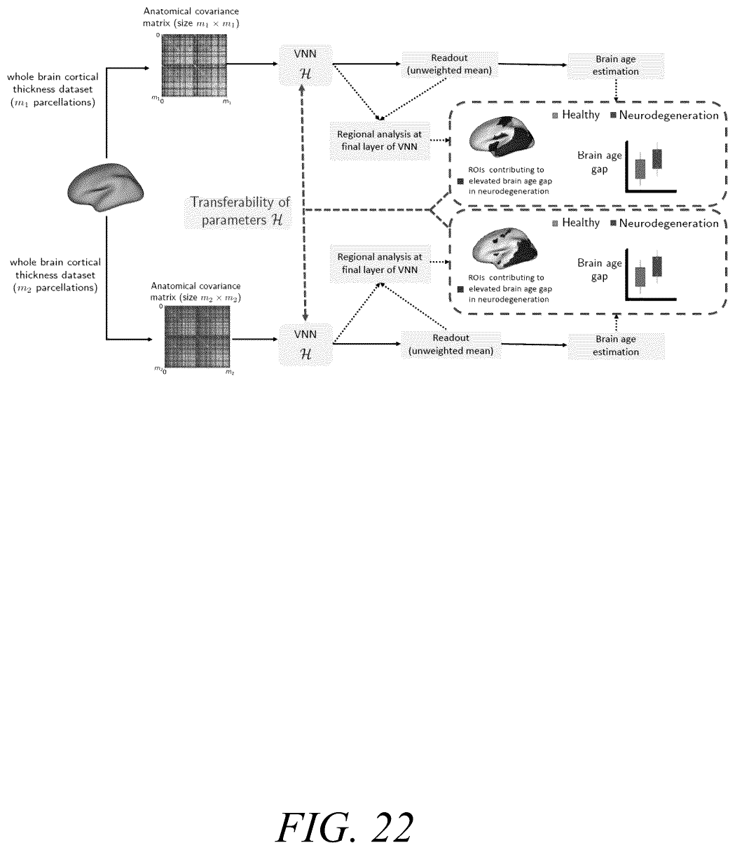

The Δ-Age for AD+ group was 3.67±3.73 years and for HC was 0±2.06 years. Hence, as expected, the Δ-Age tended to be elevated for the AD+ group. FIG. 23, Panel b displays the box plots for Δ-Age in HC and AD+ groups for FTDC100 dataset and FIG. 23, Panel c displays the scatter plots between brain age and chronological age for both groups.
2) Cross-validation on FTDC300 and FTDC500 datasets using transferability of VNNs: Next, we leverage the transferability of VNNs to cross-validate the Δ-Age results obtained via analyses of FTDC100 dataset on FTDC300 and FTDC500 datasets. For this purpose, the VNNs trained on FTDC100 dataset were transferred to predict chronological age in FTDC300 and FTDC500 datasets, followed by age-bias correction to obtain brain age and Δ-Age in both scenarios. Our approach to cross-validating Δ-Age and its associated anatomical interpretability is depicted in FIG. 22.

Due to the transferability of VNNs across FTDC datasets, Δ-Age profiles and brain age versus chronological age plots for FTDC300 and FTDC500 datasets in FIG. 21 were observed to be consistent with that for FTDC100 dataset. Hence, the transferability of VNNs enabled us to recover results similar to that of FTDC100 dataset in FTDC300 and FTDC500 datasets without re-training. This observation suggests that Δ-Age inferred by VNNs was transferable, and its anatomical interpretability was robust across different parcellations of Schaefer's atlas.

### V. Conclusions

The graph convolution operator on a covariance matrix, termed as a coVariance filter, forms the backbone of the VNN architecture. The coefficients of the coVariance filter characterize its ability to manipulate the data according to the eigenspectrum of the covariance matrix to achieve a learning objective. Thus, statistical inference using VNNs draws similarities with PCA-driven statistical approaches. However, PCA conventionally operates within the feature space of a given dataset and hence does not provide any notion of similarity between principal components of datasets with different number of features. In this paper, we have studied the key property of transferability of VNN models, which allows VNNs to be transferable between datasets with similar characteristics but different number of features. The notion of similarity between datasets consisting of different number of features is borrowed from the existing theory of graphons that studies limits of dense graphs. Specifically, our theoretical results show that if there exists a sequence of covariance matrices that converges to a continuous limit object in the limit of infinite number of features, then VNNs can be transferred between any two covariance matrices of such a sequence for statistical inference. The underlying theoretical results rely on the convergence of the eigenspectrum of a continuous approximation of covariance matrices, which result in convergence of the coVariance filter outputs for covariance matrices belonging to a converging sequence, and subsequently, the convergence of VNN outputs. Our experiments pertain to dense anatomical covariance matrices and therefore, graphon model-based analyses were certainly appropriate to study transferability of VNNs.

Furthermore, sparse covariance matrices are also of practical interest as they can help manage computational complexity. Therefore, studying VNN transferability over sparse covariance matrices is a future direction of interest. In the experiments in Section IV-C, VNNs that were trained on the healthy population were deployed on a population with AD diagnosis. Thus, in principle, VNNs learned information about healthy aging from the healthy population and were able to quantify accelerated aging as a biomarker in AD. Furthermore, the transferability of VNNs to datasets of various dimensionalities and populations in different clinical contexts draws similarities with the adaptability and transference of large-scale foundation models. The observations made here could further be extended to study VNNs trained on healthy population as foundation models for biomarkers for various health conditions in future work [55].

### Appendices

The theory of graphons has previously been leveraged to study the transferability of GNNs between graphs in the same graphon family. The proof of Theorem 2 relies on establishing the transferability of VNNs between datasets in the setting where their corresponding covariance matrices belong to a converging sequence characterized by a graphon. Our first objective in this section is to show that data processing over coVariance filter can equivalently be represented in the continuous domain using its graphon approximation. Establishing this property will ultimately allow us to compare VNNs instantiated on covariance matrices derived from datasets with different numbers of features.

Using the theory of convergence of graphons and interpreting the covariance matrix as a weighted graph representation of data, a graphon W exists as a limiting object for the sequence of graphon approximations {W_{C}_{ₘ}} if the sequence of covariance matrices {Cₘ} converges in the cut distance. A distinct feature of the cut distance is that it allows the comparison of covariance matrices of different sizes. Hence, all covariance matrices whose graphon approximations converge to a graphon can be considered to be a part of that graphon family.

Appendix a. Information Processing with Graphons

We next show that a coVariance filter H(Cₘ) can be equivalently represented in the continuous domain using convolution operations over graphon representations W_{C}_{ₘ}. Given a coVariance filter output z=H(Cₘ)x, the continuous representation of x is yₓ and that of Cₘ is W_{C}_{ₘ}. The operation Cx is fundamental to the convolution operation in H(Cₘ)x and therefore, we first provide its continuous equivalent. For s=Cx, the i-th element of s is

$${[ s ]}_{i} = {{{Σ}_{j = 0}^{m} [ {C}_{m} ]}_{i j} [ x ]}_{j} . \qquad ( 33 )$$

Thus, [s]ᵢ is a linear combination of elements in x according to the i-th row of Cₘ. In the continuous space, we can equivalently write (33) as

$${y}_{s} ( u ) = {\int }_{0}^{1} {W}_{{C}_{m}} ( u , v ) y x ( v ) dv , \qquad ( 34 )$$

where yₓ is the continuous representation of x obtained according to the intervals defined in (18). Note that yₛ is a continuous representation of s, i.e., they satisfy yₛ(u)=[s] for u∈ [symbol] ₜ. Hence, yₛ and s can be recovered from each other. This observation can be extrapolated to define the continuous equivalent of a coVariance filter. This is feasible because we can write the entity

$${C}_{m}^{k} x$$

in H(C) in a recursive form. Specifically, if we have

$${s}_{k} = {C}_{m}^{k} x ,$$

then we can rewrite sₖ as sₖ=Cₘsₖ₋₁, where s₀=x. Thus, using the same reasoning that established the equivalence between (33) and (34), we conclude that the continuous representation yₛ_{ₖ} of sₖ can be recovered via the following operation

$${y}_{{s}_{k}} ( u ) = {\int }_{0}^{1} {W}_{{C}_{m}} ( u , v ) {y}_{{s}_{k - 1}} ( v ) dv , \qquad ( 35 )$$

Since the coVariance filter output z is a weighted aggregation of the terms sₖ, we can write its continuous representation y_{z} as

$${y}_{z} ( u ) = {\sum }_{k = 0}^{K} {h}_{k} {y}_{{s}_{k}} ( u ) . \qquad ( 36 )$$

Using the mathematical steps leading up to (36), we have shown that the continuous representation of the covariance filter output z can be recovered via the convolution operations over the graphon representation W_{C}_{ₘ} in (34) and (35). Also, z and y_{z} are operationally interchangeable. Moreover, we can also extrapolate this correspondence between z and y_{z} to covariance perceptrons and VNNs with multi-layer architecture and MIMO information processing. The extension of this observation to coVariance perceptron and a basic VNN is trivial as the coVariance output is evaluated after application of pointwise non-linearity σ on z and a basic VNN is formed by stacking multiple coVariance perceptrons and number of inputs and outputs at each layer (i.e., F) being set to 1.

We use the notation xₘ to denote an input vector with m features. Thus, if VNN output Φ(xₘ; Cₘ, [symbol] ) is of size m×1 and we have F=1 and number of layers L, its continuous approximation [symbol] can be recovered by a convolutional architecture instantiated on W_{C}_{ₘ} with input yₓ_{ₘ}. For a VNN with MIMO processing, each VNN layer has multiple m-dimensional inputs and multiple m-dimensional outputs. Thus, we can equivalently define an architecture capable of performing MIMO processing that is instantiated on W_{C}_{ₘ} and xₘ and produces multiple continuous representations as the output. Such an architecture has previously been studied in the form of graphon neural networks [48]. In this context, we define the model \tilde{Φ}(yₓ_{ₘ}; W_{C}_{ₘ}, [symbol] ) that is modeled via convolution operations over W_{C}_{ₘ} in (35) and has the same architecture as the VNN Φ(xₘ; Cₘ, [symbol] ). Note that the outputs of Φ(yₓ_{ₘ}; W_{C}_{ₘ}, [symbol] ) are continuous representations of the outputs of VNN Φ(xₘ; Cₘ, [symbol] ) (see also FIG. 21 for an illustration). Thus, we can investigate the transferability of parameters [symbol] between VNNs instantiated on covariance [symbol] matrices Cₘ_{₁} and Cₘ_{₂} by analyzing the difference between

$$\tilde{Φ} ( {y}_{{x}_{{m}_{1}}} ; {W}_{{C}_{{m}_{1}}} , ℋ ) and \tilde{Φ} ( {y}_{{x}_{{m}_{2}}} ; {W}_{{C}_{{m}_{2}}} , ℋ ) .$$

In this context, our analysis hinges on the setting in which the graphon approximations

$${W}_{{C}_{{m}_{1}}} and {W}_{{C}_{{m}_{2}}}$$

belong to a sequence of graphon approximations {W_{C}_{ₘ}} that converges to a graphon W. Thus, we also consider an information processing architecture \tilde{Φ}(y; W, [symbol] ) instantiated on graphon W, such that y and continuous representations Yₓ_{ₘ} always satisfy yₓ_{ₘ}(ρᵢ)=y(ρᵢ), ∀i ∈{1, . . . , m}. Here, we can also interpret \tilde{Φ}(y; W, [symbol] ) as a generative model with \tilde{Φ}(yₓ_{ₘ}; W_{C}_{ₘ}, [symbol] ) being an instance of \tilde{Φ}(y; W, [symbol] ) at resolution m. Thus, our analysis of transferability of VNNs also includes the study of convergence of outputs from \tilde{Φ}(yₓ_{ₘ}; W_{C}_{ₘ}, [symbol] ) with that from \tilde{Φ}(y; W, [symbol] ).

To this end, we now formally define a convolution filter over a graphon and characterize its frequency response. We denote the k-hop aggregation (analogous to C^{k}x) on W_{C}_{ₘ} and continuous representation yₓ_{ₘ} by the operator

$${T}_{{W}_{{c}_{m}}}^{k} {y}_{{x}_{m}}$$

that is given by

$$( {T}_{{W}_{{c}_{m}}}^{k} {y}_{{x}_{m}} ) ( u ) \hat{=} {\int }_{0}^{1} {W}_{{C}_{m}} ( u , v ) ( {T}_{{W}_{{c}_{m}}}^{k - 1} {y}_{{x}_{m}} ) ( v ) dv , \qquad ( 37 )$$

for any k>1, where

$$( {T}_{{W}_{{c}_{m}}} {y}_{{x}_{m}} ) ( u ) \hat{=} {\int }_{0}^{1} {W}_{{C}_{m}} ( u , v ) {y}_{{x}_{m}} ( v ) dv . \qquad ( 38 )$$

Thus, based on the discussion above,

$${T}_{{W}_{{c}_{m}}}^{k} {y}_{{x}_{m}} {C}_{m}^{k} {x}_{m}$$

are operationally interchangeable. We can also define k-hop aggregation over W using the operator T_{w}y when y is related to yₓ_{ₘ} by Yₓ_{ₘ}(ρᵢ)=y(ρᵢ), where ρᵢ is defined in (18). Thus, graphon W and the continuous representation y can be seen as generative models for covariance matrix Cₘ and data point xₘ. This observation is in parallel to that in the context of graphs and graphons. We denote the graphon filter for a set of filter taps

$$ℋ = {\{ {h}_{k} \}}_{k = 0}^{K}$$

by Ψ(y; W, [symbol] ): [0,1]→ [symbol] , which is defined as

$$Ψ ( y ; W , ℋ ) ( u ) \hat{=} {\sum }_{k = 0}^{K} {h}_{k} ( {T}_{W}^{k} y ) ( u ) . \qquad ( 39 )$$

Similar to coVariance filter, the frequency response of a graphon filter can be characterized via using eigendecomposition of W in (39). Because W is bounded and symmetric, the spectral decomposition of W can be expressed as

$$W ( u , v ) = {\sum }_{i ∈ ℤ ∖ \{ 0 \}} {η}_{i} {Γ}_{i} ( u ) {Γ}_{i} ( v ) , \qquad ( 40 )$$

where ηᵢ, ∀i ∈ [symbol] \{0} are eigenvalues and Γᵢ are the eigensignals of W. Therefore, (39) can be re-stated as

$$Ψ ( y ; W , ℋ ) ( u ) = {\sum }_{i ∈ ℤ ∖ \{ 0 \}} {\sum }_{k - 1}^{K} {h}_{k} {η}_{i}^{k} {Γ}_{i} ( u ) {\int }_{0}^{1} {Γ}_{i} ( v ) y ( v ) dv \qquad ( 41 )$$

$$= {\sum }_{i ∈ ℤ ∖ \{ 0 \}} \tilde{h} ( {η}_{i} ) {Γ}_{i} ( u ) {\int }_{0}^{1} {Γ}_{i} ( v ) y ( v ) dv \qquad ( 42 )$$

for u ∈ [0,1]. Note that (42) follows from (41) using (40) and (37), and we have used the definition

$$\tilde{h} ( η ) \hat{=} {\sum }_{k = 0}^{K} {h}_{k} {η}^{k}$$

in (42). The term \tilde{h}(ηᵢ) characterizes the frequency response of a graphon filter and depends on the filter taps {hₖ} and the graphon eigenvalues.

### Appendix B. Proof of Theorem 2

In Theorem 2, we compare the continuous representations of the f-th outputs of VNNs Φ(xₘ_{₁}; Cₘ_{₁}, [symbol] ) and Φ(xₘ_{₂}; Cₘ_{₂}, [symbol] ). Our discussion in Appendix A showed that these continuous representations appear naturally as the outputs of the architectures

$$\tilde{Φ} ( {y}_{{x}_{{m}_{1}}} ; {W}_{{C}_{{m}_{1}}} , ℋ ) and \tilde{Φ} ( {y}_{{x}_{{m}_{1}}} ; {W}_{{C}_{{m}_{1}}} , ℋ )$$

instantiated on graphon approximations

$${W}_{{C}_{{m}_{1}}} and {W}_{{C}_{{m}_{2}}} ,$$

respectively. Therefore, our subsequent analysis is focused on the comparisons between their constituent graphon filters that eventually enables us to establish the convergence between f-th outputs of

$$\tilde{Φ} ( {y}_{{x}_{{m}_{1}}} ; {W}_{{C}_{{m}_{1}}} , ℋ ) and \tilde{Φ} ( {y}_{{x}_{{m}_{1}}} ; {W}_{{C}_{{m}_{1}}} , ℋ ) .$$

We begin by establishing various results pertaining to the comparisons between W and W_{C}_{ₘ}, y and yₓ_{ₘ}, and difference between eigenvalues of two distinct graphons. We leverage the (Ω, ζ)-dominant property of sequence of covariance matrices {Cₘ} in (20) and the Lipschitz condition of graphon in (21) to establish the following result.

Lemma 3. Given an α₁-Lipschitz graphon W and W_{C}_{ₘ} as graphon representation of a (Ω, ζ)-dominant covariance matrix Cₘ, we have

$${‖ W - {W}_{{C}_{m}} ‖}_{2} \leq \frac{{α}_{1} {Ω}^{\frac{3}{2}}}{{m}^{\frac{3 ζ}{2} - 1}} . \qquad ( 43 )$$

Proof. From the construction of W_{C}_{ₘ}, we have

$${‖ W - {W}_{{C}_{m}} ‖}_{2}$$

$$= {( {\int }_{0}^{1} {\int }_{0}^{1} {‖ W ( u , v ) - {W}_{{C}_{m}} ( u , v ) ‖}^{2} dudv )}^{\frac{1}{2}} \qquad ( 44 )$$

$$= {( {\sum }_{i , j ,} {\int }_{{u}_{i}} {\int }_{{u}_{i}} {‖ W ( u , v ) - {W}_{{C}_{m}} ( u , v ) ‖}^{2} dudv )}^{\frac{1}{2}} \qquad ( 45 )$$

Without loss of generality, we assume that [symbol] ₁=[0, ρ₁] is the largest interval. Using the α₁-Lipschitz continuity of graphon W_{C}_{ₘ} and noting that W_{C}_{ₘ}(ρᵢ,ρⱼ)=W(ρᵢ,ρⱼ), we have

$${‖ W - {W}_{{C}_{m}} ‖}_{2} \leq {( {\sum }_{i , j ,} {\int }_{{I}_{i}} {\int }_{{I}_{j}} {{α}_{1}^{2} ( \vert u \vert + \vert v \vert )}^{2} dudv )}^{\frac{1}{2}} \qquad ( 46 )$$

$$\leq {( {m}^{2} {\int }_{0}^{{ρ}_{1}} {\int }_{0}^{{ρ}_{1}} {{α}_{1}^{2} ( \vert u \vert + \vert v \vert )}^{2} dudv )}^{\frac{1}{2}} \qquad ( 47 )$$

$$\leq {( {m}^{2} {\int }_{0}^{{ρ}_{1}} {\int }_{0}^{{ρ}_{1}} {α}_{1}^{2} ( \vert u \vert + \vert v \vert ) dudv )}^{\frac{1}{2}} \qquad ( 48 )$$

$$\leq α m {ρ}_{1}^{3 / 2} \qquad ( 49 )$$

Using the assumption that Cₘ is (Ω, ζ)-dominant, we have

$${‖ W - {W}_{{C}_{m}} ‖}_{2} \leq \frac{{α}_{1} {Ω}^{3 / 2}}{{m}^{3 ζ / 2 - 1}} . \qquad ( 50 )$$

Next, we characterize the difference between a graphon signal y∈L₂([0,1]) and approximation yₓ_{ₘ} obtained from a random sample x in Lemma 4. For this purpose, we have the following assumption: a graphon signal y satisfies |y(a)−y(b)|≤α₂| a−b|, ∀ a, b ∈[0,1]. We term a graphon signal satisfying this property as α₂-Lipschitz graphon signal.

Lemma 4. Given an α₂-Lipschitz graphon signal y and a graphon signal approximation yₓ_{ₘ} obtained from xₘ ∈ [symbol] ^{m×1}, we have

$${‖ y - {y}_{{x}_{m}} ‖}_{2} \leq \frac{{α}_{2} {Ω}^{3 / 2}}{{m}^{3 ζ / 2 - 1}} .$$

Proof. Note that

$${‖ y - {y}_{{x}_{m}} ‖}_{2} = {\sum }_{{U}_{i}} {‖ y - {y}_{{x}_{m}} ‖}_{{L}_{2} [ {I}_{i} ]} \qquad ( 52 )$$

$$= {\sum }_{i = 1}^{m} {( {\int }_{{ρ}_{i - 1}}^{{ρ}_{i}} {( y ( u ) - {y}_{{x}_{m}} ( u ) )}^{2} du )}^{\frac{1}{2}} \qquad ( 53 )$$

where we have ρ₀=0. Using the Lipschitz property of graphon signal and (Ω, ζ)-property of Cₘ, we have

$${‖ y - {y}_{{x}_{m}} ‖}_{2} \leq {m ( {α}_{2}^{2} {\int }_{0}^{{ρ}_{1}} {u}^{2} du )}^{\frac{1}{2}} \leq \frac{{α}_{2} {Ω}^{3 / 2}}{{m}^{3 ζ / 2 - 1}} \qquad ( 54 )$$

Next, we state Proposition 4 from [14] that characterizes a bound on the difference between eigenvalues from two graphons.

Lemma 5 (Proposition 4 from). Consider two graphons W and W′ with set of eigenvalues

$${\{ {η}_{i} \}}_{i = 1}^{∞} and {\{ {β}_{i} \}}_{i = 1}^{∞} ,$$

respectively. Then, for all i∈ [symbol] ⁺, we have |ηᵢ−βᵢ|≤∥T_{W-W}, ∥₂≤∥W−W′ ∥₂.

We leverage Lemmas 3, 4, and 5 to bound the difference between graphon convolution Ψ(y; W, [symbol] ) and convolution by the approximation Ψ(yₓ_{ₘ}; W_{C}_{ₘ}, [symbol] ) realized from the coVariance filter over Cₘ.

In the following Lemma, we use the notations {\hat{η}ᵢ} and { [symbol] } for the set of eigenvalues and eigenfunctions, respectively, of W_{C}_{ₘ}. The frequency response is assumed to be band-limited, such that, |\tilde{h}(η)=0 for η≤η_{c}. Furthermore, we assume that m_{c} largest eigenvalues of graphon W in terms of magnitude satisfy |η|>η_{c} and the set of such eigenvalues is denoted by [symbol] .

Lemma 6 (Transferability of Graphon Filters). Fora convolution Ψ(y; W, [symbol] ) and its approximation Ψ(yₓ_{ₘ}; W_{C}_{ₘ}, [symbol] ), under the assumptions A1-A5 and when Lemmas 3-5 hold, we have

$${‖ Ψ ( y ; W , ℋ ) - Ψ ( {y}_{{x}_{m}} ; {W}_{{C}_{m}} , ℋ ) ‖}_{2} \leq \frac{{Ω}^{3 / 2}}{{m}^{3 ζ / 2 - 1}} ( {α}_{2} + {α}_{1} [ {α}_{3} + \frac{π {m}_{c}}{2 {Δ}_{c}} ] ) \qquad ( 55 )$$

where Δ_{c}=m [symbol] {ηᵢ−\hat{η}ⱼ} and ∥y∥₂<1. Proof. Note that we can rewrite ∥Ψ(y; W, [symbol] )−Ψ(yₓ_{ₘ}; W_{C}_{ₘ}, [symbol] )∥₂ as

$${‖ Ψ ( y ; W , ℋ ) - Ψ ( {y}_{{x}_{m}} ; {W}_{{C}_{m}} , ℋ ) ‖}_{2} = {‖ Ψ ( y ; W , ℋ ) - Ψ ( y ; {W}_{{C}_{m}} , ℋ ) + Ψ ( y ; {W}_{{C}_{m}} , ℋ ) - Ψ ( {y}_{{x}_{m}} ; {W}_{{C}_{m}} , ℋ ) ‖}_{2} \qquad ( 56 )$$

and using triangle inequality, we have

$${‖ Ψ ( y ; W , ℋ ) - Ψ ( {y}_{{x}_{m}} ; {W}_{{C}_{m}} , ℋ ) ‖}_{2} \leq {\underbrace{{‖ Ψ ( y ; W , ℋ ) - Ψ ( y ; {W}_{{C}_{m}} , ℋ ) ‖}_{2}}}_{Term 1} + {\underbrace{{‖ Ψ ( y ; {W}_{{C}_{m}} , ℋ ) - Ψ ( {y}_{{x}_{m}} ; {W}_{{C}_{m}} , ℋ ) ‖}_{2}}}_{Term 2} . \qquad ( 57 )$$

Next, we analyze Terms 1 and 2 from (57) separately.

Analysis of Term 1. Using the expansion of Ψ(y; W, [symbol] ) and Ψ(y; W_{C}_{ₘ}, [symbol] ), we have

$${‖ Ψ ( y ; W , ℋ ) - Ψ ( y ; {W}_{{C}_{m}} , ℋ ) ‖}_{2} = {( {\int }_{0}^{1} {f}^{2} ( v ) dv )}^{1 / 2} , \qquad ( 58 )$$

$$where$$

$$f ( v ) = {\sum }_{i ∈ ℤ ∖ \{ 0 \}} [ \tilde{h} ( {η}_{i} ) {Γ}_{i} ( v ) {\int }_{0}^{1} y ( u ) {Γ}_{i} ( u ) du - \tilde{h} ( {\hat{η}}_{i} ) {\hat{Γ}}_{i} ( v ) \times {\int }_{0}^{1} y ( u ) {\hat{Γ}}_{i} ( u ) du ] \qquad ( 59 )$$

By adding and subtracting

$$\tilde{h} ( {\hat{η}}_{i} ) {Γ}_{i} ( v ) {\int }_{0}^{1} y ( u ) {Γ}_{i} ( u ) du$$

in (59) and using the triangle inequality, we obtain

$${‖ Ψ ( y ; W , ℋ ) - Ψ ( y ; {W}_{{C}_{m}} , ℋ ) ‖}_{2} \leq {( {\int }_{0}^{1} {f}_{1}^{2} ( v ) dv )}^{1 {/}_{2}} + {( {\int }_{0}^{1} {f}_{2}^{2} ( v ) dv )}^{1 {/}_{2}} = {‖ {f}_{1} ‖}_{2} + {‖ {f}_{2} ‖}_{2} \qquad ( 60 )$$

$$where$$

$${f}_{1} ( v ) = {\sum }_{i ∈ ℤ ∖ \{ 0 \}} [ ( \tilde{h} ( {η}_{i} ) - \tilde{h} ( {\hat{η}}_{i} ) ) {Γ}_{i} ( v ) {\int }_{0}^{1} y ( u ) {Γ}_{i} ( u ) du ] \qquad ( 61 )$$

$$and$$

$${f}_{2} ( v ) = {\sum }_{i ∈ ℤ ∖ \{ 0 \}} [ \tilde{h} ( {\hat{η}}_{i} ) {Γ}_{i} ( v ) {\int }_{0}^{1} y ( u ) ( {Γ}_{i} ( u ) - {\hat{Γ}}_{i} ( u ) ) du ] \qquad ( 62 )$$

Using the Lipschitz property of graphon filter, we have |\tilde{h}(ηᵢ)−\tilde{h}(\hat{η}ᵢ)|≤α₃|ηᵢ−\hat{n}ᵢ|. Therefore, Lemma 5 and Lemma 3 lead to

$${‖ {f}_{1} ‖}_{2} \leq \frac{{α}_{3} {α}_{1} {Ω}^{3 / 2}}{{m}^{3 ζ / 2 - 1}} . \qquad ( 63 )$$

for any y that satisfies ∥y∥₂≤1. To analyze ∥f₂∥₂, we leverage the Cauchy-Schwarz inequality to have

$${‖ {f}_{2} ‖}_{2} \leq {\sum }_{i ∈ ℤ ∖ \{ 0 \}} \vert \tilde{h} ( {\hat{η}}_{i} ) \vert {‖ {Γ}_{i} ‖}_{2} {‖ y ( {Γ}_{i} - \hat{{Γ}_{i}} ) ‖}_{2} , \qquad ( 64 )$$

$$\leq {\sum }_{i ∈ ℤ ∖ \{ 0 \}} \vert \tilde{h} ( {\hat{η}}_{i} ) \vert {‖ {Γ}_{i} - \hat{{Γ}_{i}} ‖}_{2} , \qquad ( 65 )$$

where (64) follows from (64), without loss of generality for ∥y∥₂=1, ∥Γᵢ∥₂=1 and another application of Cauchy-Schwarz inequality. Next, we note that the integral operator T_{W}, such that,

$$( {T}_{w} y ) ( v ) = {\int }_{0}^{1} W ( u , v ) y ( u )$$

du is a self-adjoint Hilbert-Schmidt operator and W admits the spectral decomposition with {ηᵢ} as eigenvalues and {Γᵢ} as eigensignals. Therefore, to analyze ∥Γᵢ−ηᵢ∥₂, we note that Γᵢ is projection of operator T_{W} associated with eigenvalue ηᵢ and \hat{Γ}ᵢ is projection of operator

$${T}_{{W}_{{c}_{m}}}$$

associated with eigenvalue \hat{η}ᵢ. By dividing the spectrum of T_{W} as spec(T_{w})={ηᵢ}∪{ηⱼ}_{j≠1} and that of

$${T}_{{W}_{{c}_{m}}}$$

as spec

$$( {T}_{{W}_{{c}_{m}}} ) = \{ {\hat{η}}_{i} \} ⋃ {\{ {\hat{η}}_{j} \}}_{j \neq i} ,$$

we apply Proposition 2.3 from to have

$${‖ {Γ}_{i} - \hat{{Γ}_{i}} ‖}_{2} \leq \frac{π}{2} \frac{{‖ {τ}_{W} - {τ}_{{W}_{{C}_{m}}} ‖}_{2}}{{d}_{i}} , \qquad ( 66 )$$

where dᵢ>0 is a constant that satisfies |ηᵢ−\hat{η}ᵢ₊₁|≥dᵢ, |ηᵢ−\hat{η}ᵢ₋₁|≥dᵢ, |ηᵢ₊₁−\hat{η}ᵢ|≥dᵢ, and |ηᵢ₋₁−\hat{η}ᵢ|≥dᵢ. Using (66), Lemma 5 and Lemma 3 in (65), we have

$${‖ {f}_{2} ‖}_{2} \leq \frac{{παΩ}^{3 / 2}}{2 {Δ}_{c} {m}^{3 ζ / 2 - 1}} {\sum }_{i ∈ ℤ ∖ 0} \vert \tilde{h} ( {\hat{η}}_{i} ) \vert , \qquad ( 67 )$$

where Δ_{c}=minᵢdᵢ. Next, we note that |\tilde{h}(\hat{η}ᵢ)|≤1 under Assumption A5 as all eigenvalues of W_{C}_{ₘ} are smaller than or equal to 1. Hence, we have [symbol] \tilde{h}(\hat{η}ᵢ)|≤m_{c} under the band-limiting condition \tilde{h}(\hat{η}ᵢ)=0 for |ηᵢ|≤η_{c} and \tilde{h}(\hat{η}ᵢ)≠0 for at most m_{c} eigenvalues. In this scenario, we can rewrite (67) as

$${‖ {f}_{2} ‖}_{2} \leq \frac{{παΩ}^{3 / 2} {m}_{c}}{2 {Δ}_{c} {m}^{3 ζ / 2 - 1}} . \qquad ( 68 )$$

Clearly, there is a trade-off between m_{c} and ζ as we must have m_{c}<m^{3ζ/2-1} and ζ>2/3 for (68) to have decreasing behavior in m. Equations (63) and (68) provide the upper bound on Term 1.

Analysis of Term 2. We can expand term 2 as

$${‖ Ψ ( y ; {W}_{{C}_{m}} , ℋ ) - Ψ ( {y}_{{x}_{m}} ; {W}_{{C}_{m}} , ℋ ) ‖}_{2} = {( {\int }_{0}^{1} {g}^{2} ( v ) d v )}^{1 / 2} , \qquad ( 69 )$$

$$where$$

$$g ( v ) = {\sum }_{i = 1}^{∞} [ \tilde{h} ( {η}_{i} ) {\hat{Γ}}_{i} ( v ) {\int }_{0}^{1} ( y ( u ) - {y}_{{x}_{m}} ( u ) ) {\hat{Γ}}_{i} ( u ) du ] \qquad ( 70 )$$

Therefore, using (70), we have

$${‖ Ψ ( y ; {W}_{{C}_{m}} , ℋ ) - Ψ ( {y}_{{x}_{m}} ; {W}_{{C}_{m ′}} , ℋ ) ‖}_{2} = {‖ Ψ ( y - {y}_{{x}_{m}} ; {W}_{{C}_{m} ,} ℋ ) ‖}_{2} . \qquad ( 71 )$$

Note that for a frequency response that satisfies \tilde{h}(η)≤1, the graphon filter is non-expanding and therefore, we have

$${‖ Ψ ( y ; {W}_{{C}_{m} ,} ℋ ) - Ψ ( {y}_{{x}_{m}} ; {W}_{{C}_{m} ,} ℋ ) ‖}_{2} \leq {‖ y - {y}_{{x}_{m}} ‖}_{2} .$$

Using Lemma 4, we have

$$‖ Ψ ( y ; {W}_{{C}_{m}} , ℋ ) - Ψ ( {y}_{{x}_{m}} ; {W}_{{C}_{m}} , ℋ ) {‖}_{2} \leq \frac{{a}_{2} {Ω}^{3 / 2}}{{m}^{3 ζ / 2 - 1}} . \qquad ( 72 )$$

Therefore, by combining the upper bounds on Term 1 and Term 2 from (63), (68), and (73), the proof of Lemma 6 is concluded.

Lemma 6 establishes the transference between the graphon W and the graphon approximation W_{C}_{ₘ} obtained from the covariance matrix Cₘ. We leverage the result in Lemma 6 to establishing the transference for graphon neural networks in a similar setting. We denote the f-th output for graphon neural network \tilde{Ψ}(y; W_{C}_{ₘ}, [symbol] ) with F outputs in the final layer by [\tilde{Ψ}(y; W_{C}_{ₘ}, [symbol] )]_{f}.

Lemma 7 (Transferability of Graphon Neural Networks). Consider a graphon neural network \tilde{Φ}(·; W, [symbol] ) with L layers and F outputs per layer and a VNN Φ(·; Cₘ, [symbol] ) with graphon neural network representation as \tilde{Φ}(·; W_{C}_{ₘ}, [symbol] ). If the covariance matrix Cₘ belongs to a (Ω, ζ)-dominant sequence of covariance matrices and its graphon approximation W_{C}_{ₘ} belongs to a graphon family of α-Lipschitz graphon W, then under the assumptions A1-A5, for ∥y∥₂≤1 and 2/3<ζ≤1, we have

$${‖ {[ \tilde{Φ} ( y ; W , ℋ ) ]}_{f} − {[ \tilde{Φ} ( {y}_{{x}_{m}} ; {W}_{{C}_{m}} , ℋ ) ]}_{f} ‖}_{2} \qquad ( 73 )$$

$$\leq L {F}^{L} ( \frac{{Ω}^{3 / 2}}{{m}^{3 ζ / 2 - 1}} [ {α}_{2} + {α}_{1} [ {α}_{3} + \frac{π {m}_{c}}{2 {Δ}_{c}} ] ] ) . \qquad ( 74 )$$

The proof of Lemma 7 leverages Lemma 6 and accommodates the impact of multi-layer VNN architecture. We refer the reader to (23)-(28) in for exact analytical steps. Finally, by applying the triangle inequality on [14], we establish the transference between graphon neural network approximations \tilde{Φ}(·; W_{C}_{ₘ}₁, [symbol] ) and \tilde{Φ}(·; W_{C}_{ₘ}₂, [symbol] ) for VNNs Φ(·; Cₘ_{₁}, [symbol] ) and Φ(·; Cₘ_{₂}, [symbol] ), respectively, and the proof of Theorem 2 is concluded.

The disclosure of each of the following references is incorporated herein by reference in its entirety.
[1] P. Liu, “A survey of remote-sensing big data,” Front. Environ. Sci., vol. 3, p.45, 2015.
[2] B. H. Brinkmann, M. R. Bower, K. A. Stengel, G. A. Worrell, and M. Stead, “Large-scale electrophysiology: acquisition, compression, encryption, and storage of big data,” J. Neurosci. Methods, vol. 180, no. 1, pp. 185-192, 2009.
[3] J. Cai, J. Luo, S. Wang, and S. Yang, “Feature selection in machine learning: A new perspective,” Neurocomputing, vol. 300, pp. 70-79, 2018.
[4] A. Y. Ng, “Feature selection, L1 vs. L2 regularization, and rotational invariance,” in Proc. Int. Conf. Mach. Learn., 2004, pp. 78-86.
[5] S. Sihag, G. Mateos, C. McMillan, and A. Ribeiro, “coVariance neural networks,” in Proc. Conf. Adv. in Neural Inf. Process. Syst., Nov. 2022.
[6] A. C. Evans, “Networks of anatomical covariance,” Neuroimage, vol. 80, pp. 489-504, 2013.
[7] H. Murase and S. K. Nayar, “Illumination planning for object recognition using parametric eigenspaces,” IEEE Trans. Pattern Anal. Mach. Intell., vol. 16, no. 12, pp. 1219-1227, 1994.
[8] D. Stephenson, “Correlation of spatial climate/weather maps and the advantages of using the mahalanobis metric in predictions,” Tellus A, vol. 49, no. 5, pp. 513-527, 1997.
[9] H. Shao, W. H. Lam, A. Sumalee, A. Chen, and M. L. Hazelton, “Estimation of mean and covariance of peak hour origin-destination demands from day-to-day traffic counts,” Transport. Res. B-Meth., vol. 68, pp. 52-75, 2014.
[10] M. N. Ismail, A. Aborujilah, S. Musa, and A. N. Keriven, A. Bietti, and S. Vaiter, “Convergence and stability of graph convolutional networks on large random graphs,” in Proc. Conf. Adv. in Neural Inf. Process. Syst., vol. 33, 2020, pp. 21 512-21 523.
[11] S. Verma and Z. L. Zhang, “Stability and generalization of graph convolutional neural networks,” in Proc. ACM Int. Conf. Knowl. Discov. Data Min., 2019, pp. 1539-1548.
[12] N. Keriven, A. Bietti, and S. Vaiter, “Convergence and stability of graph convolutional networks on large random graphs,” in Proc. Conf. Adv. in Neural Inf. Process. Syst., vol. 33, 2020, pp. 21 512-21 523.
[13] F. Gama, J. Bruna, and A. Ribeiro, “Stability properties of graph neural networks,” IEEE Trans. Signal Process., vol. 68, pp. 5680-5695, 2020.
[14] L. Ruiz, L. Chamon, and A. Ribeiro, “Graphon neural networks and the transferability of graph neural networks,” in Proc. Conf. Adv. in Neural Inf. Process. Syst., vol. 33, 2020, pp. 1702-1712.
[15] I. T. Joliffe and B. Morgan, “Principal component analysis and exploratory factor analysis,” Stat. Methods Med. Res., vol. 1, no. 1, pp. 69-95, 1992.
[16] E. Elhaik, “Principal component analyses (PCA)-based findings in population genetic studies are highly biased and must be reevaluated,” Sci. Rep., vol. 12, no. 1, pp. 1-35, 2022.
[17] R. Levie, W. Huang, L. Bucci, M. M. Bronstein, and G. Kutyniok, “Transferability of spectral graph convolutional neural networks.” J. Mach. Learn. Res., vol. 22, pp. 272-1, 2021.
[18] Q. Zhu, C. Yang, Y. Xu, H. Wang, C. Zhang, and J. Han, “Transfer learning of graph neural networks with ego-graph inf. maximization,” in Proc. Conf. Adv. in Neural Inf. Process. Syst., vol. 34, 2021, pp. 1766-1779.
[19] S. Maskey, R. Levie, and G. Kutyniok, “Transferability of graph neural networks: An extended graphon approach,” Appl. Comput. Harmon. Anal., vol. 63, pp. 48-83, 2023.
[20] Y. Yang, Y. Zhu, H. Cui, X. Kan, L. He, Y. Guo, and C. Yang, “Data-efficient brain connectome analysis via multi-task meta-learning,” arXiv preprint arXiv:2206.04486, 2022.
[21] R. D. Markello, J. Y. Hansen, Z. Q. Liu, V. Bazinet, G. Shafiei, L. E. Suarez, N. Blostein, J. Seidlitz, S. Baillet, T. D. Satterthwaite et al., “Neuromaps: structural and functional interpretation of brain maps,” Nat. Methods, vol. 19, no. 11, pp. 1472-1479, 2022.
[22] A. Schaefer, R. Kong, E. M. Gordon, T. O. Laumann, X. N. Zuo, A. J. Holmes, S. B. Eickhoff, and B. T. Yeo, “Local-global parcellation of the human cerebral cortex from intrinsic functional connectivity MRI,” Cerebral Cortex, vol. 28, no. 9, pp. 3095-3114, 2018.
[23] P. Hagmann, L. Cammoun, X. Gigandet, R. Meuli, C. J. Honey, V. J. Wedeen, and O. Sporns, “Mapp. the structural core of human cerebral cortex,” PLoS Biol., vol. 6, no. 7, p. e159, 2008.
[24] Y. Zeighami and A. C. Evans, “Association vs. prediction: The impact of cortical surface smoothing and parcellation on brain age,” Front. Big Data, vol. 4, p. 637724, 2021.
[25] A. I. Luppi and E. A. Stamatakis, “Combining network topology and inf. theory to construct representative brain networks,” Netw. Neurosci., vol. 5, no. 1, pp. 96-124, 2021.
[26] J. Royer, R. Rodriguez-Cruces, S. Tavakol, S. Lariviere, P. Herholz, Q. Li, R. Vos de Wael, C. Paquola, O. Benkarim, B. Y. Park et al., “An open MRI dataset for multiscale neuroscience,” Sci. Data, vol. 9, no. 1, pp. 1-12, 2022.
[27] B. S. Khundrakpam, J. Tohka, A. C. Evans, B. D. C. Group et al., “Prediction of brain maturity based on cortical thickness at different spatial resolutions,” Neuroimage, vol. 111, pp. 350-359, 2015.
[28] S. Sihag, G. Mateos, C. McMillan, and A. Ribeiro, “Explainable brain age prediction using covariance neural networks,” in Proc. Conference on Neural Information Processing Systems, 2023. [Online]. Available: https://openreview.net/forum?id=cAhJF87GNO.
[29] J. H. Cole and K. Franke, “Predicting age using neuroimaging: Innovative brain ageing biomarkers,” Trends in Neurosci., vol. 40, no. 12, pp. 681-690, 2017.
[30] J. H. Cole, S. J. Ritchie, M. E. Bastin, V. Hernandez, S. Munoz Maniega, N. Royle, J. Corley, A. Pattie, S. E. Harris, Q. Zhang et al., “Brain age predicts mortality,” Mol. Psychiatry, vol. 23, no. 5, pp. 1385-1392, 2018.
[31] L. Baecker, R. Garcia-Dias, S. Vieira, C. Scarpazza, and A. Mechelli, “Machine learning for brain age prediction: Introduction to methods and clinical applications,” EBioMedicine, vol. 72, p. 103600, 2021.
[32] L. Baecker, J. Dafflon, P. F. Da Costa, R. Garcia-Dias, S. Vieira, C. Scarpazza, V. D. Calhoun, J. R. Sato, A. Mechelli, and W. H. Pinaya, “Brain age prediction: A comparison between machine learning models using region- and voxel-based morphometric data,” Human Brain Mapp., vol. 42, no. 8, pp. 2332-2346, 2021.
[33] K. Franke and C. Gaser, “Ten years of BrainAGE as a neuroimaging biomarker of brain aging: What insights have we gained?” Front. Neurol., p. 789, 2019.
[34] “Longitudinal changes in individual brain age in healthy aging, mild cognitive impairment, and Alzheimer's disease.” GeroPsych: The J. of Gerontopsychology and Geriatric Psychiatry, vol. 25, no. 4, p. 235, 2012.
[35] C. Yin, P. Imms, M. Cheng, A. Amgalan, N. F. Chowdhury, R. J. Massett, N. N. Chaudhari, X. Chen, P. M. Thompson, P. Bogdan et al., “Anatomically interpretable deep learning of brain age captures domain-specific cognitive Int. Conf. Ubiquitous Inf. Manag. Commun., 2013, pp. 1-6.
[36] A. Lombardi, D. Diacono, N. Amoroso, A. Monaco, J. M. R. Tavares, R. Bellotti, and S. Tangaro, “Explainable deep learning for personalized age prediction with brain morphology,” Frontiers in neuroscience, vol. 15, p. 578, 2021.
[37] R. J. Jirsaraie, A. J. Gorelik, M. M. Gatavins, D. A. Engemann, R. Bogdan, D. M. Barch, and A. Sotiras, “A systematic review of multimodal brain age studies: Uncovering a divergence between model accuracy and utility,” Patterns, vol. 4, no. 4, 2023.
[38] A. K. Dombrowski, M. Alber, C. Anders, M. Ackermann, K. R. Muller, and P. Kessel, “Explanations can be manipulated and geometry is to blame,” Adv. in Neural Inf. Proc. systems, vol. 32, 2019.
[39] J. Adebayo, J. Gilmer, M. Muelly, I. Goodfellow, M. Hardt, and B. Kim, “Sanity checks for saliency maps,” Adv. in neural Inf. Proc. systems, vol. 31, 2018.
[40] E. Lee, D. Braines, M. Stiffler, A. Hudler, and D. Harborne, “Developing the sensitivity of lime for better machine learning explanation,” in Artificial Intelligence and Machine Learning for Multi-Domain Operations Applications, vol. 11006. SPIE, 2019, pp. 349-356.
[41] E. Black, M. Raghavan, and S. Barocas, “Model multiplicity: Opportunities, concerns, and solutions,” in Proc. the 2022 ACM Conference on Fairness, Accountability, and Transparency, 2022, pp. 850-863.
[42] J. Chen, L. Song, M. J. Wainwright, and M. I. Jordan, “L-shapley and c-shapley: Efficient model interpretation for structured data,” arXiv preprint arXiv:1808.02610, 2018.
[43] J. Shlens, “A tutorial on principal component analysis,” arXiv preprint arXiv:1404.1100, 2014.
[44] A. Ortega, P. Frossard, J. Kovacevic, J. M. Moura, and P. Vandergheynst, “Graph signal processing: Overview, challenges, and applications,” Proc. IEEE, vol. 106, no. 5, pp. 808-828, 2018.
[45] S. Zhang, H. Tong, J. Xu, and R. Maciejewski, “Graph convolutional networks: A comprehensive review,” Comput. Soc. Netw., vol. 6, no. 1, pp. 1-23, 2019.
[46] D. I. Shuman, S. K. Narang, P. Frossard, A. Ortega, and P. Vandergheynst, “The emerging field of signal process. on graphs: Extending high-dimensional data analysis to networks and other irregular domains,” IEEE Signal Process. Mag., vol. 30, no. 3, pp. 83-98, 2013.
[47] A. Sandryhaila and J. M. Moura, “Discrete signal process. on graphs,” IEEE Trans. Signal Process., vol. 61, no. 7, pp. 1644-1656, 2013.
[48] A. Loukas, “How close are the eigenvectors of the sample and actual covariance matrices?” in Proc. Int. Conf. Mach. Learn., 2017, pp. 2228-2237.
[49] L. Ruiz, L. F. Chamon, and A. Ribeiro, “Graphon signal processing,” IEEE Trans. Signal Process., vol. 69, pp. 4961-4976, 2021.
[50] C. Borgs, J. T. Chayes, L. Lovasz, V. T. S'os, and K. Vesztergombi, “Convergent sequences of dense graphs i: Subgraph frequencies, metric properties and testing,” Adv. Math., vol. 219, no. 6, pp. 1801-1851, 2008.
[51] L. Lovasz, Large Networks and Graph Limits. American Mathematical Soc., 2012, vol. 60.
[52] S. M. Lundberg and S. I. Lee, “A unified approach to interpreting model predictions,” Adv. in Neural Inf. Proc. Syst., vol. 30, 2017.
[53] M. T. Ribeiro, S. Singh, and C. Guestrin, ““why should i trust you?” explaining the predictions of any classifier,” in Proc. the 22nd ACM SIGKDD international conference on knowledge discovery and data mining, 2016, pp. 1135-1144.
[54] N. J. Tustison, P. A. Cook, A. Klein, G. Song, S. R. Das, J. T. Duda, B. M. Kandel, N. van Strien, J. R. Stone, J. C. Gee et al., “Large-scale evaluation of ANTs and Freesurfer cortical thickness measurements,” Neuroimage, vol. 99, pp. 166-179, 2014.
[56] S. R. Das, B. B. Avants, M. Grossman, and J. C. Gee, “Registration based cortical thickness measurement,” Neuroimage, vol. 45, no. 3, pp. 867-879, 2009.
[57] N. Alon and A. Naor, “Approximating the cut-norm via Grothendieck's inequality,” SIAM J. Comput., vol. 35, no. 4, pp. 787-803, 2006.
[58] A. Paszke, S. Gross, F. Massa, A. Lerer, J. Bradbury, G. Chanan, T. Killeen, Z. Lin, N. Gimelshein, L. Antiga et al., “Pytorch: An imperative style, high-performance deep learning library,” in Proc. Conf. Adv. in Neural Inf. Process. Syst., vol. 32, 2019.
[59] T. Akiba, S. Sano, T. Yanase, T. Ohta, and M. Koyama, “Optuna: A next-generation hyperparameter optimization framework,” in Proc. ACM SIGKDD Int. Conf. Knowl. Discov. Data Min., 2019, pp. 2623-2631.
[60] M. Jove, M. Portero-Otin, A. Naudi, I. Ferrer, and R. Pamplona, “Metabolomics of human brain aging and age-related neurodegenerative diseases,” J. Neuropathol. Exp. Neurol., vol. 73, no. 7, pp. 640-657, 2014.
[61] T. Schafer and C. Ecker, “fsbrain: an R package for the visualization of structural neuroimaging data,” Biorxiv, pp. 2020-09, 2020.
[62] I. Beheshti, S. Nugent, O. Potvin, and S. Duchesne, “Bias-adjustment in neuroimaging-based brain age frameworks: A robust scheme,” Neuroimage Clin., vol. 24, p. 102063, 2019.
[63] A. M. G. de Lange and J. H. Cole, “Commentary: Correction procedures in brain-age prediction,” Neuroimage Clin., vol. 26, 2020.
[64] J. Bien and R. J. Tibshirani, “Sparse estimation of a covariance matrix,” Biometrika, vol. 98, no. 4, pp. 807-820, 2011.
[65] A. Dosovitskiy, L. Beyer, A. Kolesnikov, D. Weissenborn, X. Zhai, T. Unterthiner, M. Dehghani, M. Minderer, G. Heigold, S. Gelly et al., “An image is worth 16×16 words: Transformers for image recognition at scale,” in International Conference on Learning Representations, 2020.
[66] A. Seelmann, “Notes on the sin 20 theorem,” Integral Equ. Oper. Theory., vol. 79, no. 4, pp. 579-597, 2014.

It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A method for identifying biomarkers indicative of neurodegeneration using a covariance neural network (VNN), the method comprising:
providing a VNN trained to predict features informative of chronological age using an anatomical covariance matrix and brain anatomical data derived from a population comprising a largest portion of healthy subjects;
providing, as input to the VNN, brain anatomical data of a subject and the anatomical covariance matrix;
generating, by the VNN and based on the input, a set of biomarkers indicative of neurodegeneration of the subject; and
generating, based on the set of biomarkers generated by the VNN, a brain health marker indicative of neurodegeneration of the subject.

2. The method of claim 1 wherein the brain anatomical data used to train the VNN comprises multivariate dataset, whose each element captures a characteristic of a brain region or a combination of brain regions, and is derived from at least one of: magnetic resonance imaging (MRI) images, computed tomography (CT) scan, positron emission tomography (PET) scan, electroencephalogram (EEG) test of brains of a population comprising healthy subjects.

3. The method of claim 1 wherein the VNN is trained for predicting chronological age or features informative of the chronological age.

4. The method of claim 1 wherein the brain anatomical data of the subject comprises a multivariate dataset, whose each element captures a characteristic of a brain region or a combination of brain regions and is derived from a combination of at least two of: magnetic resonance imaging (MRI) images, computed tomography (CT) scan, positron emission tomography (PET) scan, electroencephalogram (EEG) test of brains.

5. The method of claim 1 wherein the brain anatomical data of the subject captures information about the same section of the brain as the brain anatomical data used to train the VNN.

6. The method of claim 1 wherein elements of the anatomical covariance matrix used to train the VNN are determined by covariance between the features associated with different brain regions or a transformation of the covariance between the features associated with different brain regions. Examples of transformations on the covariance between the features associated with different brain regions can include thresholding, division, normalization, and multiplication.

7. The method of claim 1 wherein elements of the anatomical covariance matrix used to process the brain anatomical data of the subject are determined by the covariance between the features associated with different brain regions or a transformation of the covariance between the features associated with different brain regions. Examples of transformations on the covariance between the features associated with different brain regions can include thresholding, division, normalization, and multiplication.

8. The method of claim 1 wherein the anatomical covariance matrix used to process the brain anatomical data of the subject may have different number of features relative to the anatomical covariance matrix used to train the VNN.

9. The method of claim 1 wherein generating the biomarker indicative of neurodegeneration of the subject includes generating the biomarker as outputs of the VNN or statistical transformation of the outputs of the VNN.

10. The method of claim 1 wherein generating the brain health marker indicative of neurodegeneration of the subject comprises determining, from the biomarkers, a prediction of brain age of the subject.

11. The method of claim 1 wherein generating the brain health marker indicative of neurodegeneration of the subject comprises determining, from the biomarkers, a label of the subject among a category representing healthy population and one or more categories representing populations with neurodegenerative health conditions.

12. The method of claim 1 comprising mapping anatomical regions of the subject's brain to the biomarker and identifying anatomical regions of the subject's brain contributing to brain age of the subject.

13. The method of claim 12 wherein identifying the anatomical regions contributing to the brain age includes evaluating a statistic for an anatomical region from the biomarker that characterizes the anatomical region with respect to the brain age determined from the biomarkers.

14. The method of claim 13 wherein identifying the anatomical regions of the subject's brain contributing to the brain age comprises identifying the anatomical regions contributing to a prediction of brain age of the subject.

15. The method of claim 12 wherein identifying the anatomical regions contributing to the brain age includes statistically comparing the biomarker for the subject relative to the biomarkers of a healthy population.

16. A system for identifying biomarkers indicative of neurodegeneration using a covariance neural network (VNN), the system comprising:
a computing platform including at least one processor, a memory, and a VNN trained exclusively on brain anatomical data from a dataset comprising brain anatomical derived from a population comprising a largest portion of healthy subjects, with the VNN implemented by the at least one processor for:
receiving brain anatomical data of a subject as input;
generating, based on the input, a set of biomarkers indicative of neurodegeneration of the subject; and
generating, based on the set of biomarkers, a brain health marker indicative of neurodegeneration of the subject.

17. The system of claim 16 wherein the brain anatomical data used to train the VNN comprises a multivariate dataset, whose each element captures a characteristic of a brain region or a combination of brain regions, and is derived from a combination of two or more of: magnetic resonance imaging (MRI) images, computed tomography (CT) scan, positron emission tomography (PET) scan, electroencephalogram (EEG) test of brains of a population comprising healthy subjects.

18. The system of claim 16 wherein elements of an anatomical covariance matrix used to train the VNN are determined by covariance between features associated with different brain regions or a transformation of the covariance between the features associated with different brain regions.

19. The system of claim 16 wherein generating the set of biomarkers indicative of neurodegeneration of the subject includes generating the set as outputs of the VNN or a statistical transformation of the outputs of the VNN.

20. The system of claim 16 wherein generating the brain health marker indicative of neurodegeneration of the subject includes a linear or non-linear transformation of the biomarkers.

21. The system of claim 16 wherein generating the brain health marker indicative of neurodegeneration of the subject includes generating brain age based on a linear or non-linear aggregation of the biomarkers.

22. The system of claim 16 comprising identifying anatomical regions of the subject's brain contributing to brain age of the subject by evaluating a residual vector for each anatomical region from the biomarker generated by the VNN that characterizes the anatomical region.

23. The system of claim 16 comprising mapping anatomical regions of the subject's brain to the biomarkers.

24. The system of claim 16 comprising identifying anatomical regions of the subject's brain contributing to the brain health marker.

25. The system of claim 16 wherein generating the brain health marker indicative of neurodegeneration of the subject includes prediction of the subject or the brain of the subject being healthy or unhealthy.

26. The system of claim 16 comprising identifying anatomical regions of the subject's brain contributing to prediction of the subject or the brain of the subject being healthy or unhealthy.

27. A non-transitory computer readable medium having stored thereon a VNN trained exclusively on brain anatomical data derived from a population comprising a largest portion of healthy subjects and executable instructions that when executed by at least one processor of at least one computer cause the at least one computer to perform steps comprising:
receiving brain anatomical data of a subject as input;
generating, based on the input, a set of biomarkers indicative of neurodegeneration of the subject; and
generating, based on the input, a brain health marker indicative of neurodegeneration of the subject.

28. The non-transitory computer readable medium of claim 27 wherein generating the brain health marker indicative of neurodegeneration of the subject includes generating a prediction of brain age of the subject.

29. The non-transitory computer readable medium of claim 27 wherein generating the brain health marker indicative of neurodegeneration of the subject includes predicting the subject or the brain of the subject as healthy or unhealthy.
